(19) United States
(12) Patent Application Publication
(10) Pub. No.: US 2023/0172877 A1
(43) Pub. Date: Jun. 8, 2023

(54) METABOLITES RELEASED FROM APOPTOTIC CELLS ACT AS NOVEL TISSUE MESSENGERS

(71) Applicant: University of Virginia Patent Foundation, Charlottesville, VA (US)

(72) Inventors: Kodimangalam S. Ravichandran; Christopher B. Medina

(73) Assignee: University of Virginia Patent Foundation, Charlottesville, VA (US)

(21) Appl. No.: 17/910,613

(22) PCT Filed: Mar. 11, 2021
(86) PCT No.: PCT/US2021/021983

§ 371 Date: Sep. 9, 2022

Related U.S. Application Data
(60) Provisional application No. 62/988,188, filed on Mar. 11, 2020.

(51) Int. Cl. A61K 31/132; A61K 31/661; A61P 29/00

(52) U.S. Cl. CPC A61K 31/132; A61K 31/661; A61P 29/00

(57) ABSTRACT

Disclosed aspects include characterizing of the metabolite secretome of apoptotic cells, deciphering metabolite-based communication between dying cells and neighboring live cells, and harnessing components of the secretome for beneficial effects in vivo. In representative embodiments, a composition comprising, consisting essentially of, or consisting of an effective amount of a plurality of metabolite compounds derived from an apoptotic cell is disclosed, as are methods of treating an inflammatory condition in a subject and modulating gene expression in a subject using the composition.

### CROSS REFERENCE TO RELATED APPLICATIONS

This application claims benefit of U.S. Provisional Pat. Application Serial No. 62/988,188, filed Mar. 11, 2020, herein incorporated by reference in its entirety.

### GOVERNMENT INTEREST

This invention was made with government support under Grant Nos. HL120840 and GM122542 awarded by The National Institutes of Health. The government has certain rights in the invention.

### TECHNICAL FIELD

The presently disclosed subject matter relates to assessing apoptotic metabolites. More particularly, the presently disclosed subject matter relates to assessing metabolites released from apoptotic cells and related methods and compositions.

### BACKGROUND

Apoptosis occurs during development³, homeostatic tissue turnover, and pathological settings¹. Besides the known responses of phagocytes that engulf apoptotic cells⁴, the apoptotic process itself (independent of phagocytosis), can modulate physiological events, such as embryogenesis and tissue regeneration⁵, with pathologies arising when apoptosis is inhibited⁶. However, the mechanisms by which apoptotic cells themselves mediate these functions are incompletely understood. As apoptotic cells remain intact for a period of time, they could release soluble metabolites that diffuse within a tissue to influence neighboring cells. Although a few soluble factors from apoptotic cells are reported as ‘find-me’ signals to attract phagocytes⁷, the full apoptotic secretome is not yet defined.

### SUMMARY

This Summary lists several embodiments of the presently disclosed subject matter, and in many cases lists variations and permutations of these embodiments of the presently disclosed subject matter. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In some embodiments, the presently disclosed subject matter provides a method of treating an inflammatory condition in a subject. In some embodiments, the method comprising administering to the subject an effective amount of a plurality of metabolite compounds derived from an apoptotic cell, to thereby treat the inflammatory condition in the subject. In some embodiments, the inflammatory condition is selected from the group comprising arthritis, transplantation rejection, colitis, peritonitis, and atherosclerosis. In some embodiments, the administering of the plurality of metabolite compounds derived from an apoptotic cell induces an anti-inflammatory response in a macrophage, a myeloid cell, a non-professional phagocyte, or any combination thereof.

In some embodiments, the plurality of metabolite compounds derived from an apoptotic cell are formulated in a single composition. In some embodiments, the plurality of metabolite compounds derived from an apoptotic cell are formulated in a pharmaceutically acceptable carrier. In some embodiments, the plurality of metabolite compounds comprises three or more metabolite compounds derived from an apoptotic cell. In some embodiments, the metabolite compound is selected from the group comprising spermidine, fructose 1,6-bisphosphate (FBP), dihydroxyacetone phosphate (DHAP), guanosine 5′-monophosphate (GMP), inosine 5′-monophosphate (IMP), and UDP-glucose and combinations thereof.

In some embodiments, the presently disclosed subject matter provides a method of modulating gene expression in a cell. In some embodiments, the method comprises contacting the cell with an effective amount of a plurality of metabolite compounds derived from an apoptotic cell, to thereby modulate gene expression in the cell. In some embodiments, the cell is a cell in a subject. In some embodiments, the gene expression is involved in a biological process selected from the group consisting of inflammation, wound healing, proliferation, and development.

In some embodiments, the plurality of metabolite compounds derived from an apoptotic cell are formulated in a single composition. In some embodiments, the plurality of metabolite compounds derived from an apoptotic cell are formulated in a pharmaceutically acceptable carrier. In some embodiments, the plurality of metabolite compounds comprises three or more metabolite compounds derived from an apoptotic cell. In some embodiments, the metabolite compound is selected from the group comprising spermidine, fructose 1,6-bisphosphate (FBP), dihydroxyacetone phosphate (DHAP), guanosine 5′-monophosphate (GMP), inosine 5′-monophosphate (IMP), and UDP-glucose and combinations thereof.

In some embodiments, the presently disclosed subject matter provides a composition comprising, consisting essentially of, or consisting of an effective amount of a plurality of metabolite compounds derived from an apoptotic cell; and a carrier. In some embodiments, the carrier is a pharmaceutically acceptable carrier.

In some embodiments, the plurality of metabolite compounds comprises three or more metabolite compounds derived from an apoptotic cell. In some embodiments, the metabolite compound is selected from the group comprising spermidine, fructose 1,6-bisphosphate (FBP), dihydroxyacetone phosphate (DHAP), guanosine 5′-monophosphate (GMP), inosine 5′-monophosphate (IMP), and UDP-glucose and combinations thereof.

In some embodiments, the composition is for use in treating an inflammatory condition, for use in preparing a medicament for treating an inflammatory condition; for use in modulating gene expression in a cell; and/or for use in preparing a medicament for modulating gene expression in a cell. In some embodiments, the inflammatory condition is selected from the group comprising arthritis, transplantation rejection, colitis, peritonitis, and atherosclerosis. In some embodiments, the composition induces an anti-inflammatory response in a macrophage, a myeloid cell, a non-professional phagocyte, or any combination thereof. In some embodiments, the gene expression is involved in a biological process selected from the group consisting of inflammation, wound healing, proliferation, and development.

In some embodiments, the presently disclosed subject matter provides a method of classifying cell death. In some embodiments, the method comprises providing a sample to be assessed; detecting in the sample a presence or an absence of a profile comprising a plurality of metabolite compounds; and classifying cell death in the sample based on the presence or the absence of the profile. In some embodiments, the profile comprises a plurality of metabolite compounds derived from an apoptotic cell. In some embodiments, the presence or the absence of the profile indicates whether the predominant type of cell death in the sample is apoptosis or is not apoptosis. In some embodiments, the plurality of metabolite compounds comprises three or more metabolite compounds derived from an apoptotic cell. In some embodiments, the profile comprises a metabolite compound selected from the group consisting of spermidine, fructose 1,6-bisphosphate (FBP), dihydroxyacetone phosphate (DHAP), guanosine 5′-monophosphate (GMP), inosine 5′-monophosphate (IMP), and UDP-glucose and combinations thereof.

Accordingly, it is an object of the presently disclosed subject matter to provide for the assessment of metabolites released from apoptotic cells and related methods and compositions. This and other objects are achieved in whole or in part by the presently disclosed subject matter. Further, objects of the presently disclosed subject matter having been stated above, other objects and advantages of the presently disclosed subject matter will become apparent to those skilled in the art after a study of the following description, Figures, and EXAMPLES. Additionally, various aspects and embodiments of the presently disclosed subject matter are described in further detail below.

### BRIEF DESCRIPTION OF THE DRAWINGS

FIGS. 1A to 1F show conserved metabolite secretome from apoptotic cells. FIG. 1A, Schematic for assessing apoptotic metabolite secretomes. FIG. 1B, Venn diagrams illustrating the ‘shared’ apoptotic metabolites identified across cell types, modalities of apoptosis induction, and the two metabolomic platforms tested, and the list of five shared metabolites plus ATP. FIGS. 1C, 1D, 1E, Metabolite release from Jurkat T cells (n=3 for ATP-UV, Spermidine-UV+zVAD, Spermidine-ABT, and Spermidine-Fas. n=4 for ATP-ABT, ATP-Fas, and Spermidine-Fas-live. n=5 for Spermidine-UV-live and Spermidine-Fas+zVAD), A549 lung epithelial cells (n=3), and HCT-116 colonic epithelial cells (n=3) across different apoptotic stimulus with or without caspase inhibition with zVAD. The key for the three different gray shades goes from left to right in the same order as the corresponding bars for each measurement. FIG. 1F, Several abundant metabolites such as (i) alanine, (ii) pyruvate, and (iii) creatinine were not released in the Jurkat T cell supernatants (n=4) (* p < 0.05, ** p < 0.01, *** p < 0.001, **** p <0.0001). Data are mean ± s.e.m (c-e), Data are mean ± s.d (e). Unpaired Student’s t-test with Holm-Sidak method for multiple t-tests.

FIGS. 2A to 2F show Panx1 activation and continued metabolic activity of dying cells orchestrates metabolite release. FIG. 2A, Panxl-dependent metabolite release. Heatmap produced from untargeted metabolomics of Jurkat T cell supernatants representing the metabolites that were statistically enriched in the apoptotic supernatants relative to live supernatant (p < 0.05), and reduced when Panx1 was inhibited via a Panxl-DN (genetic), and two Panx1 inhibitors (Spiro and Trovan) (pharmacologic) (p < 0.05). Metabolites are grouped by pathway. Charge and relative sizes (Da) of the metabolites are also shown (n=4). Two-sided Welch’s two-sample t-test. Color version shows enriched in red and reduced in blue.

FIG. 2B, Three-way Venn diagram (left) illustrating the eight Panxl-dependent apoptotic metabolites observed among the cell types and apoptotic modalities tested. ATP (not detected here) represents the 9^{th} metabolite. FIG. 2C, Supernatant spermidine concentration per million cells (targeted metabolomics) from Jurkats (Fas crosslinking - 4 hours - left) (n=3) (***p=0.0002) or primary thymocytes with Panx1 deletion (Fas - 1.5 hours - right) (n=3) (****p=0.0001). FIG. 2D, (left) Schematic of the polyamine metabolic pathway. (right) Relative amounts of ornithine, putrescine, and spermidine in Jurkat T cell supernatants in live and apoptotic conditions, with or without Panx1 inhibition (n=4) (****p=0.0001) FIGS. 2E-2F, Active polyamine metabolic activity during apoptosis. Experimental layout for ¹³C-arginine labeling (FIG. 2F), and incorporation of ¹³C-labeled arginine into the polyamine pathway intermediates putrescine (FIG. 2F-left) (***p=0.0003) and spermidine (FIG. 2F-right) (*p=0.025) after cell death induction (n=6). Data are mean ± s.e.m. Ordinary One-way ANOVA, Turkey’s multiple comparison test (FIG. 2C, FIG. 2D). Unpaired Student’s t-test with Holm-Sidak method for multiple t-tests (FIG. 2E, FIG. 2F).

FIGS. 3A to 3D show metabolites from apoptotic cells influence gene programs in live cells. FIG. 3A, Schematic for assessing gene induction by apoptotic cell supernatants in LR73 cells. FIG. 3B, Gene expression programs induced in phagocytes by the apoptotic secretome. Display shows the differentially regulated genes (1852 total, 886 upregulated, 966 downregulated), categorized per known or predicted function(s), literature, and sequence similarity. Circle size is proportional to the number of differentially expressed genes (n=4) (Significance <0.05). Color version shows upregulated in red and downregulated in blue. FIG. 3C, Differentially regulated genes in phagocytes in response to apoptotic cell supernatants with or without pannexin channel inhibition (82 upregulated, darker gray or darker purple in color, 28 downregulated, lighter grey or lighter purple in color) (n=4). FIG. 3D, Validation of genes regulated by Panxl-dependent metabolites. LR73 cells were incubated with indicated supernatants for 4 hr, gene expression of Areg (n=7) (****p=0.0001), Nr4al(n=7) (Live-AC ****p=0.0001, AC-AC Panxl-DN ***p=0.0008), Uapl(n=4) (****p=0.0001), and Pbxl (n=5) (Live-AC **p=0.009, AC-AC Panxl-DN *p=0.014) expression in phagocytes. Data are mean ± s.e.m. Ordinary One-way ANOVA, Turkey’s multiple comparison test.

FIGS. 4A to 4H show Panxl-dependent metabolite release during apoptosis modulates phagocyte gene expression in vivo and can alleviate inflammation. FIG. 4A, Panx1 expression in apoptotic thymocytes influences gene expression in myeloid cells in vivo. Control mice (Panxl^{ƒl/ƒl}, no Cre) or mice lacking Panx1 in thymocytes (Panxl^{ƒl/ƒl} Cd4-Cre) were injected with dexamethasone to induce apoptosis in thymocytes (Panxl^{ƒl/ƒl} Cd4-Cre- PBS n=3, Panxl^{ƒl/ƒl} Cd4-Cre- Dex n=6, Panxl^{ƒl/ƒl} Cd4-Cre+ PBS n=4, Panxl^{ƒl/ƒl} Cd4-Cre+ Dex n=4). After 6 hr, CD11b⁺ CD1 1c⁺ phagocytes were purified for mRNA. qPCR analysis of Uapl (WT PBS-WT Dex *p=0.032, WT Dex-KO Dex ****p<0.0001), Pbxl (WT PBS-WT Dex ****p=0.0001, WT Dex-KO Dex *p=0.0103), and Ugdh (****p<0.0001) in CD11b+CD11c+ phagocytes. FIG. 4B, Panxl-dependent metabolites released from apoptotic cells were compared across cell types and apoptotic conditions to design different metabolite mixtures, Memix⁶ (light grey, blue when shown in color) and Memix³ (dark grey, purple when shown in color). FIG. 4C, MeMix⁶ (n=6) andMemix³ (n=4) solutions mimic gene expression changes in phagocytes induced by apoptotic supernatants (*p<0.05, **p<0.01, ****p<0.0001). FIG. 4D, Schematic of arthritis induction and treatments (top). Paw swelling was measured using a caliper and reported as % change compared to day 0 (MeMix⁶ **p=0.0028, MeMix³ ***p=0.0003). Scores were assessed on a scale of 1-4 per paw (MeMix⁶ ***p=0.0004, MeMix³ ****p=0.0001) (Vehicle n=16, MeMix⁶ n=11, MeMix³ n=12). FIG. 4E, Ankle inflammation and bone erosion were scored via H&E staining (left) and Safranin O (right), respectively, from arthritic mouse paws. Increased magnifications of affected areas are also shown. FIG. 4F, Clinical analysis of inflammation, bone erosion, and cartilage erosion was scored by an investigator blinded to treatments (Vehicle n=6, MeMix³ n=7) (****p<0.0001). FIG. 4G, MeMix³ metabolite solution alleviates inflammation in a minor antigen-mismatch lung transplant model. Orthotopic left lung transplantation from C57BL/10 mice into C57BL/6 recipient mice, with Memix3 administered on post-operation day 1 and 3. Lungs were harvested for histological scoring on day 7. FIG. 4H, H&E staining (left) and ISHLT Rejection score (right) (Vehicle n=6, MeMix³ n=6) (*p=0.024). Data are mean ± s.e.m. (FIG. 4A, FIG. 4C, FIG. 4D). Data are mean ± s.d. (FIG. 4F, FIG. 4H). Ordinary One-way ANOVA, Turkey’s multiple comparison test (FIG. 4A). Unpaired two-tailed Student’s t-test (FIG. 4C, FIG. 4F, FIG. 4H). Two-Way ANOVA (FIG. 4D).

FIGS. 5A to 5D show metabolite release from apoptotic Jurkat cells. FIG. 5A, Jurkat cells were induced to undergo apoptosis after UV irradiation. Staining with 7AAD and Annexin V (AV) were used to determine the percentage of live (AV⁻7AAD⁻), apoptotic (AV⁺7AAD⁻), or necrotic (AV⁺7AAD⁺) cells after 4 hours. FIG. 5B, Quantitative analysis of apoptosis (top) and secondary necrosis (bottom) (n=4). Data are mean ± s.d. Unpaired two-tailed Student’s t-test. **** p< 0.0001. FIG. 5C, Volcano Plot produced from untargeted metabolomics of Jurkat T cell supernatants representing statistically enriched or reduced (p < 0.05) metabolites in the apoptotic supernatants relative to live supernatant. Data are representative of four biological replicates. Two-sided Welch’s two-sample t-test. Color version shows enriched in red (predominantly right side of plot) and reduced in blue (predominantly left side of plot). FIG. 5D, Heatmap produced from untargeted metabolomics of Jurkat T cell supernatants representing statistically enriched or reduced (p < 0.05) metabolites in the apoptotic supernatants relative to live supernatants. Data are representative of four biological replicates. Two-sided Welch’s two-sample t-test. Color version shows enriched in red (predominantly top right and lower left) and reduced in blue (predominantly top left and lower right).

FIGS. 6A to 6F show reciprocal metabolite changes between apoptotic supernatant and pellet. FIG. 6A, Heatmap produced from untargeted metabolomics of Jurkat T cell pellets representing statistically enriched or reduced (p< 0.05) metabolites in the apoptotic pellet relative to live cell pellet (n=4 biologically independent samples). Two-sided Welch’s two-sample t-test. Color version shows enriched in red (predominantly top right and lower left) and reduced in blue (predominantly top left and lower right). FIG. 6B, Bi-directional plot representing the 85 metabolites that were statistically enriched in the apoptotic supernatant (p < 0.05) and simultaneously reduced in the apoptotic cell pellet relative to live cell conditions. Metabolites were grouped by metabolic pathways (n=4 biologically independent samples). Two-sided Welch’s two-sample t-test. FIGS. 6C-6F, Mass spectrometry was used to determine the relative amount of (FIG. 6C) spermidine. (**** p< 0.0001), (FIG. 6D) inosine (**** p< 0.0001), (FIG. 6E) UDP-glucose (supernatant **** p< 0.0001, pellet *p=0.014), and (FIG. 6F) AMP (**** p< 0.0001) in Jurkat T cell supernatants and cell pellets in live and apoptotic conditions (n=4 biologically independent samples). Data are mean ± s.d. Unpaired two-tailed Student’s t-test.

FIGS. 7A to 7C show conserved metabolite release during apoptosis. FIG. 7A, Mass spectrometry was used to measure the concentration of the of the five metabolites that were released across all conditions and platforms tested in live or apoptotic supernatants per million Jurkat T cells (left) or isolated primary thymocytes (right) (back-calculated from total cells used in experimental set-up) (n=3). Metabolites are grouped by pathways to which they have been linked. Data are mean ± s.d. Unpaired two-tailed Student’s t-test. Thymocyte-creatine *p=0.014, Jurkat-spermidine **p=0.0014, Thymocyte-glycerol-3-phosphate ***p=0.0002, **** p < 0.0001. FIG. 7B, Luciferase assay was used to measure the concentration of ATP release in the supernatant across the different apoptotic Jurkat cells (n=4). Data are mean ± s.e.m. Ordinary One-way ANOVA, Turkey’s multiple comparison test. **** p < 0.0001. FIG. 7C, Table outlining the different cell types, apoptotic stimulus, techniques and metabolites screened for Untargeted (>3000 features/compounds) and Targeted (116 metabolites) metabolomics included ATP, Spermidine, Glycerol-3-phosphate (G-3-P) and creatine.

FIGS. 8A to 8B show Panx1 activation and inhibition during cell death. FIG. 8A, Representative histograms of TO-PRO-3 dye uptake (top) as a readout of Panx1 activation in live and apoptotic thymocytes from wild type (Panxl^{+/+}) and Panx1 knockout (Panxl^{-/-}) mice. Quantification of Panx1 activation across different conditions was assessed via flow cytometry by measuring the mean fluorescent intensity of TO-PRO-3 dye uptake (bottom) (n=3). Data are mean ± s.e.m. Ordinary One-way ANOVA, Turkey’s multiple comparison test. **** p <0.0001. FIG. 8B, Representative histograms of TO-PRO-3 dye uptake as a readout of Panx1 activation in live and apoptotic wild-type Jurkat T cells, Panxl-DN Jurkat T cells, and Jurkat T cells treated with two different Panx1 inhibitors spironolactone (50 µM) or trovafloxacin (25 µM) (top). Quantification of TO-PRO-3 dye uptake by the apoptotic cells measured as MFI assessed using flow cytometry (bottom) (n=4). Data are mean ± s.e.m. Ordinary One-way ANOVA, Turkey’s multiple comparison test. **** p < 0.0001.

FIGS. 9A to 9E show Panx1 inhibition does not influence apoptotic cell death. FIG. 9A, Control or Panxl^{-/-}thymocytes were treated with anti-Fas (5 µg ml⁻¹) for 1.5 hours. Cells were stained with 7AAD and Annexin V to determine the percentage of live (AV⁻7AAD⁻ ), apoptotic (AV⁺7AAD⁻), or necrotic (AV⁺7AAD⁺) cells. FIG. 9B, Quantitation of apoptosis (top) and secondary necrosis (bottom) of control and Panx1 knockout thymocytes (n=3). Data are mean ± s.e.m. Ordinary One-way ANOVA, Turkey’s multiple comparison test. ***p=0.0004. FIG. 9C, FIG. 9D, Quantification of apoptosis and secondary necrosis from the samples prior to metabolomics analysis (n=4). Data are mean ± s.e.m. Ordinary One-way ANOVA, Turkey’s multiple comparison test. ****p < 0.0001. FIG. 9E, Cells were stained with 7AAD and Annexin V to determine the percent of live (AV⁻7AAD⁻), apoptotic (AV⁺7AAD⁻), or necrotic (AV⁺7AAD⁺) cells.

FIGS. 10A to 10E show Panxl-dependent metabolite release during apoptosis. FIG. 10A, Mass spectrometry was used to determine the relative amount of AMP, GMP, UDP-Glucose, and fructose 1,6-bisphosphate in Jurkat T cell supernatant across different conditions (n=4). Data are mean ± s.e.m. Ordinary One-way ANOVA, Turkey’s multiple comparison test. **** p < 0.0001. FIG. 10B, Jurkat cells were induced to undergo apoptosis with anti-Fas treatment (250 ng ml⁻¹). Mass spectrometry was used to measure the absolute concentration per million cells of (a) AMP, (b) UDP-glucose, and (c) fructose 1,6-bisphosphate in the supernatants of Jurkat T cells across different conditions (back-calculated from total cells used in experimental set-up) (n=3). Data are mean ± s.e.m. Ordinary One-way ANOVA, Turkey’s multiple comparison test. (UDP-Glucose Live vs. No Txn **p=0.0013, No Txn vs. Panxl-DN *p=0.031, **** p< 0.0001). FIG. 10C, Mass spectrometry was used to determine the absolute concentration of AMP, GMP, UDP-Glucose, and fructose 1,6-bisphosphate per million cells (back-calculated from total cells used in experimental set-up) in the supernatant from isolated primary thymocytes across different conditions (n=3). Data are mean ± s.e.m. Ordinary One-way ANOVA, Turkey’s multiple comparison test. **** p< 0.0001. FIG. 10D-FIG. 10E, Relative concentrations were determined by mass spectrometry for inosine (d) and choline (e) in live, apoptotic, or apoptotic supernatants where Panx1 was inhibited (n=4). Data are mean ± s.e.m. Ordinary One-way ANOVA, Turkey’s multiple comparison test. **** p<0.0001.

FIG. 11 shows conserved Panx1 secretome. Three-way Venn diagram (top) comparing Panxl-dependent metabolites released from apoptotic cells across different conditions tested. Table (bottom) showing the relative peak intensity (untargeted metabolomics) or absolute concentrations (targeted metabolomics) in the supernatant of the indicated cell treatments and knockout mice.

FIGS. 12A to 12C show transcriptional and metabolic changes during apoptosis. FIG. 12A, Re-analyses of RNA-seq data from apoptotic cells (Lui et. al., 2018) demonstrating that the SRM mRNA levels are increased/retained during apoptosis. Color version shows increased in red (predominantly middle right and far right) and decreased in blue (predominantly middle left and far left). FIG. 12B, After induction of apoptosis (n=4), the SRM mRNA expression was assessed over time relative to live controls (n=5). Data are mean ± s.e.m. Two-way ANOVA (**p=0.007). FIG. 12C, Incorporation of ¹³C-labeled arginine into the polyamine pathway intermediate spermidine and release from Jurkat cells after apoptosis, and its partial reduction by the pan-caspase inhibitor zVAD (n=3). Data are mean ± s.d. Unpaired two-tailed Student’s t-test (**p=0.0088).

FIGS. 13A to 13C show transcriptional changes on surrounding phagocytes induced by Panxl-dependent metabolite release during apoptosis. FIG. 13A, Principle component analysis (PCA) on the RNAseq data as a quality control statistic (n=4 biological replicates). FIG. 13B, Experimental procedure was as described in FIG. 3D. qPCR was used to assess gene expression changes in Ptgs2 (top) (****p<0.0001) and Sgk1 (middle) (Live-AC **p=0.0074, AC-AC-PanxlDN **p=0.0031) ( and Slcl4al (bottom) (****p<0.0001) in phagocytes after treatment with supernatants from Jurkat cells or Jurkat cells expressing dominant negative Panx1 (n=7). Data are mean ± s.e.m. Ordinary One-way ANOVA, Turkey’s multiple comparison test. FIG. 13C, Experimental procedure was as described in FIG. 3D, however before treatment of LR73 cells with supernatant, the supernatant was filtered through a 3 kDa filter to remove large molecules. qPCR was used to assess gene expression changes in and Sgk1 (top) (***p=0.0001) and Slcl4al (bottom) (****p<0.0001) in phagocytes after treatment with supernatants under specified conditions (n=3). Data are mean ± s.e.m. Ordinary One-way ANOVA, Turkey’s multiple comparison test.

FIGS. 14A to 14F show in vivo thymic cell death analysis and supernatant effects during arthritis. FIG. 14A, Analysis of thymic populations used for experimental data presented in FIG. 4A. After thymus isolation, the CD11b⁻CD11c⁻population which contained thymocytes was used for mRNA isolation to test the efficiency of deletion Panx1 allele. qPCR analysis of Panx1 mRNA in wild-type (Panxl^{ƒl/ƒl} CD4-Cre⁻) (n=6) or mice in which Panx1 has been knocked out in thymocytes (Panxl^{ƒl/ƒl}CD4-Cre⁺) (n=7) (left) (**p=0.0015). CD11b+/c+ myeloid cells harvested from the thymus of Panxlƒl/ƒlCd4-Cre -/+ were analyzed for Panx1 expression to demonstrate that Panx1 not deleted. Panx1 deletion was only deleted from thymocytes and not the myeloid cells which do not express CD4. Data are mean ± s.d. Unpaired two-tailed Student’s t-test. FIG. 14B, Representative flow cytometric plots showing the extent of apoptosis induced by dexamethasone in control and Panxl^{ƒl/ƒl} CD4-Cre⁺ mice. After thymus isolation, an aliquot of cells was stained with 7AAD and Annexin V to determine the % of live, apoptotic, or necrotic (AV⁺7AAD⁺) cells. FIG. 14C, Quantitative analysis of apoptosis (left) and secondary necrosis (right) of CD11b⁻CD11c⁻ thymic populations from Panxl^{ƒl/ƒl} CD4-Cre⁻ (PBS n=4, Dex n=10) or Panxl^{ƒl/ƒl} CD4-Cre⁺ (PBS n=3, Dex n=9) mice treated with PBS or dexamethasone. Data are mean ± s.e.m. Ordinary One-way ANOVA, Turkey’s multiple comparison test (****p<0.0001). FIG. 14D, Representative flow cytometry plots demonstrating the purity of CD11b⁺CD11c⁺ population after magnetic separation from the different mice and treatment conditions. FIG. 14E, Comparison of the CD11b+CD11c+ cells isolated from the different conditions. Panxl^{ƒl/ƒl} CD4-Cre⁻ (PBS n=4, Dex n=7) or Panxl^{ƒl/ƒl} CD4-Cre⁺ (PBS n=3, Dex n=6). Data are mean ± s.e.m. Ordinary One-way ANOVA, Turkey’s multiple comparison test. FIG. 14F, Apoptotic supernatants alleviate KBx/N induced arthritic disease. C57B1/6J mice were injected with K/BxN serum to induce arthritis. Live (n=4) or apoptotic (n=5) supernatant was given for five days after arthritis induction. Paw swelling was measured using a caliper and reported as % change compared to day 0. Data are mean ± s.e.m. Two-way ANOVA (*p=0.0131).

### DETAILED DESCRIPTION

Caspase-dependent apoptosis accounts for about 90% of homeostatic cell turnover in the body¹, and regulates inflammation, cell proliferation, and tissue regeneration²⁻⁴. How apoptotic cells mediate such diverse effects is not fully understood. The presently disclosed subject matter profiled the apoptotic “metabolite secretome” and addressed their effects on the tissue neighborhood. Apoptotic lymphocytes and macrophages release specific metabolites, while retaining their membrane integrity. A subset of these metabolites is also shared across different primary cells and cell lines after apoptosis induction by different stimuli. Mechanistically, apoptotic metabolite secretome was not due to passive emptying of contents, rather orchestrated. First, caspase-mediated opening of the plasma membrane Pannexin 1 channels facilitated release of a select subset of the metabolite secretome. Second, certain metabolic pathways continue to remain active during apoptosis, with release of select metabolites from a given pathway. Functionally, the apoptotic metabolite secretome induced specific gene programs in healthy neighboring cells, including suppression of inflammation, cell proliferation, and wound healing. Further, a cocktail of select apoptotic metabolites reduced disease severity in mouse models of inflammatory arthritis and lung graft rejection.

Among the billions of cells that turn over in the body every day as part of routine homeostasis, greater than 90% of death occurs via apoptosis. Even after a cell commits to apoptosis, it often takes minutes to hours to fully execute the apoptotic program. During this interim period, the apoptotic cells maintain plasma membrane integrity and can communicate with neighboring cells via regulated release of small molecules such as metabolites. Recently, inter-cellular communication through metabolites as extracellular signaling molecules is emerging as an exciting new area of research. Despite many years of apoptosis research, only of a handful of molecules released from apoptotic cells have been identified, primarily as “find-me” signals that induce chemotaxis of phagocytes (e.g. ATP, LPC). The full composition of metabolites that form part of the “apoptotic secretome” has not yet been determined. In accordance with aspects of the presently disclosed subject matter, the “metabolite secretome” from apoptotic cells is defined, and the functionality of members of the metabolite secretome is tested in vitro and in vivo in regulating neighboring live cells.

Collectively, the data and disclosure presented herein advance several concepts. As a first, non-limiting example, specific metabolites that are released from apoptotic cells (different cell types and modes of apoptosis induction) were identified; the specificity could arise from metabolic changes in the apoptotic cells (e.g., sustained spermidine production), and/or the opening of specific channels (e.g., Panxl). As a second, non-limiting example, it was discovered that apoptotic cells are not inert awaiting removal; rather, via metabolites as “good-bye” signals modulate multiple gene programs in the neighboring cells within a tissue. As a third, non-limiting example, the ability of a cocktail of apoptotic metabolites to attenuate arthritic symptoms and lung transplantation rejection shows that it is possible to harness the beneficial therapeutic properties of apoptosis in specific inflammatory conditions. Methods, therapeutics, and related compositions for treatment and/or mitigation of inflammatory conditions are therefore provided in accordance with some embodiments of the presently disclosed subject matter.

### I. Definitions

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the presently disclosed and claimed subject matter.

Following long-standing patent law convention, the terms “a”, “an”, and “the” refer to “one or more” when used in this application, including in the claims. For example, the phrase “an antibody” refers to one or more antibodies, including a plurality of the same antibody. Similarly, the phrase “at least one”, when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to whole number values between 1 and 100 and greater than 100.

Unless otherwise indicated, all numbers expressing quantities of ingredients, reaction conditions, and so forth used in the specification and claims are to be understood as being modified in all instances by the term “about”. The term “about”, as used herein when referring to a measurable value such as an amount of mass, weight, time, volume, concentration, or percentage, is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods and/or employ the disclosed compositions. Accordingly, unless indicated to the contrary, the numerical parameters set forth in this specification and attached claims are approximations that can vary depending upon the desired properties sought to be obtained by the presently disclosed subject matter.

A disease or disorder is “alleviated” if the severity of a symptom of the disease, condition, or disorder, or the frequency at which such a symptom is experienced by a subject, or both, are reduced.

As used herein, the term “and/or” when used in the context of a list of entities, refers to the entities being present singly or in combination. Thus, for example, the phrase “A, B, C, and/or D” includes A, B, C, and D individually, but also includes any and all combinations and subcombinations of A, B, C, and D.

As used herein, the term “adjuvant” refers to a substance that elicits an enhanced immune response when used in combination with a specific antigen.

As use herein, the terms “administration of” and/or “administering” a compound should be understood to refer to providing a compound of the presently disclosed subject matter to a subject in need of treatment.

The term “comprising”, which is synonymous with “including” “containing”, or “characterized by”, is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting essentially of” limits the scope of the related disclosure or claim to the specified materials and/or steps, plus those that do not materially affect the basic and novel characteristic(s) of the disclosed and/or claimed subject matter. For example, a pharmaceutical composition can “consist essentially of” a pharmaceutically active agent or a plurality of pharmaceutically active agents, which means that the recited pharmaceutically active agent(s) is/are the only pharmaceutically active agent(s) present in the pharmaceutical composition. It is noted, however, that carriers, excipients, and/or other inactive agents can and likely would be present in such a pharmaceutical composition, and are encompassed within the nature of the phrase “consisting essentially of”.

As used herein, the phrase “consisting of” excludes any element, step, or ingredient not specifically recited. It is noted that, when the phrase “consists of” appears in a clause of the body of a claim, rather than immediately following the preamble, it limits only the element set forth in that clause; other elements are not excluded from the claim as a whole.

With respect to the terms “comprising”, “consisting of”, and “consisting essentially of”, where one of these three terms is used herein, the presently disclosed and claimed subject matter can include the use of either of the other two terms. For example, a composition that in some embodiments comprises a given active agent also in some embodiments can consist essentially of that same active agent, and indeed can in some embodiments consist of that same active agent.

The term “aqueous solution” as used herein can include other ingredients commonly used, such as sodium bicarbonate described herein, and further includes any acid or base solution used to adjust the pH of the aqueous solution while solubilizing a peptide.

The term “binding” refers to the adherence of molecules to one another, such as, but not limited to, enzymes to substrates, ligands to receptors, antibodies to antigens, DNA binding domains of proteins to DNA, and DNA or RNA strands to complementary strands.

“Binding partner”, as used herein, refers to a molecule capable of binding to another molecule.

The term “biocompatible”, as used herein, refers to a material that does not elicit a substantial detrimental response in the host.

As used herein, the terms “biologically active fragment” and “bioactive fragment” of a peptide encompass natural and synthetic portions of a longer peptide or protein that are capable of specific binding to their natural ligand and/or of performing a desired function of a protein, for example, a fragment of a protein of larger peptide which still contains the epitope of interest and is immunogenic.

The term “biological sample”, as used herein, refers to samples obtained from a subject, including but not limited to skin, hair, tissue, blood, plasma, cells, sweat, and urine. The biological sample can include a biological fluid, for example, but not limited to, follicular fluid, seminal plasma, uterine lining fluid, urine, plasma, blood, spinal fluid, serum, interstitial fluid, sputum, saliva.

A “coding region” of a gene comprises the nucleotide residues of the coding strand of the gene and the nucleotides of the non-coding strand of the gene which are homologous with or complementary to, respectively, the coding region of an mRNA molecule which is produced by transcription of the gene.

“Complementary” as used herein refers to the broad concept of subunit sequence complementarity between two nucleic acids (e.g., two DNA molecules). When a nucleotide position in both of the molecules is occupied by nucleotides normally capable of base pairing with each other at a given position, the nucleic acids are considered to be complementary to each other at this position. Thus, two nucleic acids are complementary to each other when a substantial number (in some embodiments at least 50%) of corresponding positions in each of the molecules are occupied by nucleotides that can base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. By way of example and not limitation, the first region comprises a first portion and the second region comprises a second portion, whereby, when the first and second portions are arranged in an antiparallel fashion, in some embodiments at least about 50%, in some embodiments at least about 75%, in some embodiments at least about 90%, and in some embodiments at least about 95% of the nucleotide residues of the first portion are capable of base pairing with nucleotide residues in the second portion. In some embodiments, all nucleotide residues of the first portion are capable of base pairing with nucleotide residues in the second portion.

A “compound”, as used herein, refers to a polypeptide, an isolated nucleic acid, a metabolite, or other agent used in the methods of the presently disclosed subject matter.

A “control” cell, tissue, sample, or subject is a cell, tissue, sample, or subject of the same type as a test cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. The control may also be obtained from another source or similar source other than the test group or a test subject, where the test sample is obtained from a subject suspected of having a condition, disease, or disorder for which the test is being performed.

A “test” cell or subject is a cell or subject being examined.

A “pathogenic” cell or tissue is a cell or tissue that causes or contributes to a condition, disease, or disorder in the animal in which the cell or tissue is located (or from which the tissue was obtained).

A tissue “normally comprises” a cell if one or more of the cell are present in the tissue in an animal not afflicted with a condition, disease, or disorder.

As used herein, the terms “condition”, “disease condition”, “disease”, “disease state”, and “disorder” refer to physiological states in which diseased cells or cells of interest can be targeted and/or assessed with the compositions and methods of the presently disclosed subject matter. In some embodiments, a condition is an inflammatory condition. In some embodiments, the inflammatory condition is selected from the group comprising arthritis, transplantation rejection, colitis, peritonitis, and atherosclerosis. As used herein, the term “diagnosis” refers to detecting a risk or propensity to a condition, disease, or disorder. In any method of diagnosis exist false positives and false negatives. Any one method of diagnosis does not provide 100% accuracy.

A “disease” is a state of health of an animal wherein the animal cannot maintain homeostasis, and wherein if the disease is not ameliorated then the animal’s health continues to deteriorate.

In contrast, a “disorder” in an animal is a state of health in which the animal is able to maintain homeostasis, but in which the animal’s state of health is less favorable than it would be in the absence of the disorder. Left untreated, a disorder does not necessarily cause a further decrease in the animal’s state of health.

As used herein, an “effective amount” or “therapeutically effective amount” refers to an amount of a compound or composition sufficient to produce a selected effect, such as but not limited to alleviating symptoms of a condition, disease, or disorder. In the context of administering compounds in the form of a combination, such as multiple compounds, the amount of each compound, when administered in combination with one or more other compounds, may be different from when that compound is administered alone. Thus, an effective amount of a combination of compounds refers collectively to the combination as a whole, although the actual amounts of each compound may vary. The term “more effective” means that the selected effect occurs to a greater extent by one treatment relative to the second treatment to which it is being compared.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA, and mRNA) or a defined sequence of amino acids and the biological properties resulting therefrom. Thus, a gene encodes a protein if transcription and translation of an mRNA corresponding to or derived from that gene produces the protein in a cell or other biological system and/or an in vitro or ex vivo system. Both the coding strand, the nucleotide sequence of which is identical to the mRNA sequence (with the exception of uracil bases presented in the latter) and is usually provided in Sequence Listing, and the non-coding strand, used as the template for transcription of a gene or cDNA, can be referred to as encoding the protein or other product of that gene or cDNA.

A “fragment”, “segment”, or “subsequence” is a portion of an amino acid sequence, comprising at least one amino acid, or a portion of a nucleic acid sequence comprising at least one nucleotide. The terms “fragment”, “segment”, and “subsequence” are used interchangeably herein.

As used herein, a “functional” biological molecule is a biological molecule in a form in which it exhibits a property by which it can be characterized. A functional enzyme, for example, is one that exhibits the characteristic catalytic activity by which the enzyme can be characterized.

As used herein “injecting”, “applying”, and administering” include administration of a compound of the presently disclosed subject matter by any number of routes and modes including, but not limited to, topical, oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, vaginal, and rectal approaches.

As used herein, a “ligand” is a compound that specifically binds to a target compound or molecule. A ligand “specifically binds to” or “is specifically reactive with” a compound when the ligand functions in a binding reaction which is determinative of the presence of the compound in a sample of heterogeneous compounds.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, such as but not limited to through ionic or hydrogen bonds or van der Waals interactions.

The terms “measuring the level” and “determining the level” as used herein refer to any measure or assay which can be used to correlate the results of the assay with the level or amount of a compound of interest, such as a metabolite compound. Such assays are described in the examples and also include spectroscopic, northern and western blot analyses, binding assays, immunoblots, etc. The terms “measuring the level of expression” and “determining the level of expression” as used herein refer to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present. Such assays are coupled with processes or systems to store and process information and to help quantify levels, signals, etc. and to digitize the information for use in comparing levels.

The term “otherwise identical sample”, as used herein, refers to a sample similar to a first sample, that is, it is obtained in the same manner from the same subject from the same tissue or fluid, or it refers a similar sample obtained from a different subject. The term “otherwise identical sample from an unaffected subject” refers to a sample obtained from a subject not known to have the disease or disorder being examined. The sample may of course be a standard sample. By analogy, the term “otherwise identical” can also be used regarding regions or tissues in a subject or in an unaffected subject.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

The term “pharmaceutical composition” refers to a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

“Pharmaceutically acceptable” means physiologically tolerable, for either human or veterinary application. Similarly, “pharmaceutical compositions” include formulations for human and veterinary use.

As used herein, the term “pharmaceutically acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Plurality” means at least two.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” refers to non-naturally occurring peptides or polypeptides. Synthetic peptides or polypeptides can be synthesized, for example, using an automated polypeptide synthesizer. Various solid phase peptide synthesis methods are known to those of skill in the art.

The term “prevent”, as used herein, means to stop something from happening, or taking advance measures against something possible or probable from happening. In the context of medicine, “prevention” generally refers to action taken to decrease the chance of getting a disease or condition. It is noted that “prevention” need not be absolute, and thus can occur as a matter of degree.

A “preventive” or “prophylactic” treatment is a treatment administered to a subject who does not exhibit signs, or exhibits only early signs, of a condition, disease, or disorder. A prophylactic or preventative treatment is administered for the purpose of decreasing the risk of developing pathology associated with developing the condition, disease, or disorder.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

As used herein, the term “purified” and like terms relate to an enrichment of a molecule or compound, or combination of compounds, relative to other components normally associated with the molecule or compound or combination of compounds in a native environment. The term “purified” does not necessarily indicate that complete purity of the particular molecule, compound, or combination of compounds has been achieved during the process.

A “highly purified” compound, or combination of compounds, as used herein refers to a compound or combination of compounds that is in some embodiments greater than 90% pure, that is in some embodiments greater than 95% pure, and that is in some embodiments greater than 98% pure.

As used herein, the term “mammal” refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

The term “subject” as used herein refers to a member of species for which treatment and/or prevention of a disease or disorder using the compositions and methods of the presently disclosed subject matter might be desirable. Accordingly, the term “subject” is intended to encompass in some embodiments any member of the Kingdom Animalia including, but not limited to the phylum Chordata (e.g., members of Classes Osteichythyes (bony fish), Amphibia (amphibians), Reptilia (reptiles), Aves (birds), and Mammalia (mammals), and all Orders and Families encompassed therein.

The compositions and methods of the presently disclosed subject matter are particularly useful for warm-blooded vertebrates. Thus, in some embodiments the presently disclosed subject matter concerns mammals and birds. More particularly provided are compositions and methods derived from and/or for use in mammals such as humans and other primates, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), rodents (such as mice, rats, and rabbits), marsupials, and horses. Also provided is the use of the disclosed methods and compositions on birds, including those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, also provided is the use of the disclosed methods and compositions on livestock, including but not limited to domesticated swine (pigs and hogs), ruminants, horses, poultry, and the like.

A “sample”, as used herein, refers in some embodiments to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

The term “standard”, as used herein, refers to something used for comparison. For example, it can be a known standard reagent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

As used herein, a “subject in need thereof” is a patient, animal, mammal, or human, or other subject, who will benefit from the methods and compositions of this presently disclosed subject matter.

The term “substantially pure” describes a particular compound, or combination of compounds, e.g. metabolite, small molecule, nucleic acid, protein and/or peptide, which has been separated from components which naturally accompany it. Typically, a compound or combination of compounds is substantially pure when in some embodiments at least 10%, in some embodiments at least 20%, in some embodiments at least 50%, in some embodiments at least 60%, in some embodiments at least 75%, in some embodiments at least 90%, and in some embodiments at least 99% of the total material (by volume, by wet or dry weight, or by mole percent or mole fraction) in a sample is the compound of interest or combination of compounds of interest. Purity can be measured by any appropriate method, e.g., in the case of polypeptides by column chromatography, gel electrophoresis, or HPLC analysis. A compound or combination of compounds, e.g., a metabolite or a plurality of metabolites, is also substantially purified when it is essentially free of naturally associated components or when it is separated from the native contaminants which accompany it in its natural state.

The term “symptom”, as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse, and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

As used herein, the phrase “therapeutic agent” refers to an agent that is used to, for example, treat, inhibit, prevent, mitigate the effects of, reduce the severity of, reduce the likelihood of developing, slow the progression of, and/or cure, a disease or disorder.

The terms “treatment” and “treating” as used herein refer to both therapeutic treatment and prophylactic or preventative measures, wherein the object is to prevent or slow down (lessen) the targeted pathologic condition, prevent the pathologic condition, pursue or obtain beneficial results, and/or lower the chances of the individual developing a condition, disease, or disorder, even if the treatment is ultimately unsuccessful. Those in need of treatment include those already with the condition as well as those prone to have or predisposed to having a condition, disease, or disorder, or those in whom the condition is to be prevented.

As used herein, the terms “vector”, “cloning vector”, and “expression vector” refer to a vehicle by which a polynucleotide sequence (e.g., a foreign gene) can be introduced into a host cell, so as to transduce and/or transform the host cell in order to promote expression (e.g., transcription and translation) of the introduced sequence. Vectors include plasmids, phages, viruses, etc.

All genes, gene names, and gene products disclosed herein are intended to correspond to homologs and/or orthologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates.

All references listed in the instant disclosure, including but not limited to all patents, patent applications and publications thereof, scientific journal articles, and database entries (including but not limited to UniProt, EMBL, and GENBANK® biosequence database entries and including all annotations available therein) are incorporated herein by reference in their entireties to the extent that they supplement, explain, provide a background for, and/or teach methodology, techniques, and/or compositions employed herein. The discussion of the references is intended merely to summarize the assertions made by their authors. No admission is made that any reference (or a portion of any reference) is relevant prior art. Applicants reserve the right to challenge the accuracy and pertinence of any cited reference.

### II. Representative Embodiments

### II. A. Generally

In some aspects, the presently disclosed subject matter advances the concept that apoptotic cells are not ‘inert corpses’ waiting for removal, but rather release metabolites as ‘good-bye’ signals that actively modulate tissue outcomes.

### Profiling the Metabolite Secretome of Apoptotic Cells

Using unbiased metabolomics, it was demonstrated that apoptotic cells release metabolites into the extracellular milieu. Further, an “apoptotic secretome” that includes many metabolites not previously known to be released from dying cells was characterized. Further, a metabolite signature shared across apoptotic T cell lines and primary thymocytes was defined, across different apoptotic triggers. Further, mechanistically, it was shown that apoptotic cells are not passively releasing their contents. First, a specific subset of the apoptotic secretome is released via pannexin channels, which are activated/opened by caspase-mediated apoptosis. Second, apoptotic cells continue to maintain their activity in certain metabolic pathways, and the release of select metabolites from these pathways.

### Apoptotic Metabolites Influence the Tissue Environment

Using a combination of RNAseq and in vivo approaches (where Panx1 is specifically deleted in apoptotic thymocytes), it was determined that apoptotic metabolites control specific gene programs and networks in neighboring cells. Thus, “natural” and “physiologically-derived” extracellular metabolites can regulate genes involved in processes such as inflammation regulation, wound healing, proliferation, and development. Thus, the metabolite secretome of apoptotic cells can significantly impact the tissue environment.

### Harnessing the Anti-Inflammatory Power of a Specific Subset of Metabolites

Many therapies for inflammatory diseases involve synthetic compounds that can have toxic side effects. By systematically narrowing down from the total the apoptotic cell secretome, a specific subset of metabolites was defined. Administering a cocktail of these metabolites into mice strongly attenuated disease parameters in a model of inflammatory arthritis and in a model of lung transplant rejection.

Collectively, these studies progress from defining the metabolite secretome of apoptotic cells, to deciphering metabolite-based communication between dying cells and neighboring live cells including phagocytes, and to harnessing components of the secretome for beneficial effects in vivo.

### Representative Therapeutic Implementations

Apoptotic metabolites are anti-inflammatory and can be used for dampening inflammatory diseases. As few as three of these metabolites can be used in vivo to reduce the symptoms of arthritis and in a lung transplantation model. While some of these metabolites (such as spermidine and FBP) are known to have anti-inflammatory properties, no one has ever shown that apoptotic cells can serve as a novel source for these metabolites. Further, it is shown that one can mix them into a cocktail to induce potent anti-inflammatory responses in macrophages and myeloid cells, as well as non-professional phagocytes. Thus, the impact of using this metabolite cocktail is quite powerful for specific inflammatory diseases.

### II.B Representative Profiles, Methods, and Compositions of the Presently Disclosed Subject Matter

An “apoptotic secretome” profile that includes many metabolites not previously known to be released from dying cells is provided in accordance with the presently disclosed subject matter. In some embodiments, the profile encompasses a plurality of metabolite compounds present in an apoptotic cell, present in a sample comprising an apoptotic cell, released in a medium or tissue where an apoptotic cell is present, or any combination thereof. In some embodiments, the profile is used to assess an aspect of the health of a subject from which the profile is identified. Also, the profile can be used to classify cell death in a cell or tissue, such as in a particular disease tissues or disorder tissues, as subsequent therapeutic strategies impinge on the type of death occurring within that tissue /disease context and/or tissue/disorder context. This information can be used to assess a subject’s health and to assess therapy options for a subject having a particular disease and/or disorder. Profiles include the presence of a metabolite compound and/or a level of a metabolite compound.

In some embodiments, the plurality of metabolite compounds comprises three or more metabolite compounds derived from an apoptotic cell. Representative apoptotic cell types and metabolite compounds are described in the Examples, Tables, and Figures. Examples of metabolite compounds include but are not limited to sugars, fatty acids, amino acids, nucleotides, intermediates formed during cellular processes, and other small molecules found in vivo. Additional cell types of interest relevant for particular disease contexts include but are not limited to: apoptotic intestinal epithelial cells (in the context of colitis), apoptotic macrophages and stromal cells within atherosclerotic plaques (atherosclerosis), and apoptotic adipocytes (obesity). In some embodiments, the metabolite compound is selected from the group comprising spermidine, fructose 1,6-bisphosphate (FBP), dihydroxyacetone phosphate (DHAP), guanosine 5′-monophosphate (GMP), inosine 5′-monophosphate (IMP), and UDP-glucose and combinations thereof.

In some embodiments, the presently disclosed subject matter provides a method for classifying cell death in a cell or tissue. By way of elaboration and not limitation, there are many approaches already existing to track cell death via different modalities (such as apoptosis, pyroptosis, necroptosis, and the like.). However, currently available approaches require that the tissue be removed, stained, or otherwise tested for activation of specific proteins in these samples to fully gauge the type of death. Even then, the approaches can be inconclusive as the tissue damage itself causes death. However, the metabolites that are released during these different modalities of cell death differ. That is, the apoptotic secretome is different from the pyroptotic secretome, for example. Therefore, in accordance with aspects of the presently disclosed subject matter, by taking a sample, such as but not limited to a small aliquot of fluid or tissue from a subject and screening for particular metabolites indicates whether the predominant type of death happening in a cell or tissue (such as in a disease context and/or disorder context) is apoptosis or is not apoptosis, but rather could be pyroptosis, necroptosis, and/or the like.

In some embodiments, a method of classifying cell death comprises providing a sample, such as a sample comprising a cell or tissue to be assessed; detecting in the sample a presence or an absence of a profile comprising a plurality of metabolite compounds; and classifying cell death in the sample based on the presence or the absence of the profile. In some embodiments, the profile comprises a plurality of metabolite compounds derived from an apoptotic cell. In some embodiments, the presence or absence of the profile is used to determine that the predominant type of death happening in a cell or tissue (such as in a disease context and/or disorder context) is apoptosis or is not apoptosis. “Absence” does not necessarily mean the profile is completely undetectable, but it can mean this. For example, “absence” can also mean that certain metabolite compounds within a profile might be present, but certain other metabolite compounds within a profile might not be present at detectible levels or might be present at negligible levels. Correspondingly, the “presence” of the profile can indicate that each of the members of the plurality of metabolite compounds are present, although the levels of each of the members of the plurality of metabolite compounds can vary. Approaches for detecting profiles and metabolic compound components thereof are described elsewhere herein, including the EXAMPLES, Figures, and Tables.

In some embodiments, the method further comprises assessing the health of the subject based on the classifying of the cell death in the cell or tissue from the sample from the subject and/or determining a treatment step for the subject based on the classifying of cell death. In some embodiments, the subject is suffering from a disease and/or disorder characterized by an inflammatory condition. In some embodiments, the inflammatory condition is selected from the group comprising arthritis, transplantation rejection, colitis, peritonitis, and atherosclerosis. Patient health can also include as a condition, including healthy, or stressed, wherein stressed includes, for example, but not limited to, obese, pregnant, anorexic, bulemic, cachexic, diabetic, including more than one body disorder; and cancer including, including more than one type of cancer.

In some embodiments, the presently disclosed subject matter provides a computer readable medium, encoded with instructions for carrying out a method for classifying cell death in a cell or tissue. In some embodiments, the presently disclosed subject matter provides a computer system, comprising: an input/output device; a processor; and a memory, wherein the memory is configured with instructions, executable by the processor, to carry out a method for classifying cell death in a cell or tissue, and to provide the results of the method to a user, via the input/output device.

In some embodiments, the presently disclosed subject matter provides a method of treating an inflammatory condition in a subject. In some embodiments, the method comprising administering to the subject an effective amount of a plurality of metabolite compounds derived from an apoptotic cell, to thereby treat the inflammatory condition in the subject. In some embodiments, the inflammatory condition is selected from the group comprising arthritis, transplantation rejection, colitis, peritonitis, and atherosclerosis. In some embodiments, the administering of the plurality of metabolite compounds derived from an apoptotic cell induces an anti-inflammatory response in a macrophage, a myeloid cell, a non-professional phagocyte, or any combination thereof.

In some embodiments, the presently disclosed subject matter provides a method of modulating gene expression in a cell. In some embodiments, the method comprises contacting the cell with an effective amount of a plurality of metabolite compounds derived from an apoptotic cell, to thereby modulate gene expression in the cell. In some embodiments, the cell is a cell in a subject. In some embodiments, the gene expression is involved in a biological process selected from the group consisting of inflammation, wound healing, proliferation, and development.

By the phrase “derived from an apoptotic cell” it is meant that the plurality of metabolite compounds are associated with, found in and/or purified from an apoptotic cell, associated with, found in and/or purified from a sample comprising an apoptotic cell, associated with, found in and/or purified from a medium or tissue where an apoptotic cell is present, and/or associated with, found in and/or purified from any other source of apoptotic cells as would be apparent to one of ordinary skill in the art upon a review of the present disclosures. Additional approaches for deriving metabolic compounds from apoptotic cells, as well as examples of types of apoptotic cells are provided in the EXAMPLES, Figures, and Tables set forth herein below. Additional cell types of interest relevant for particular disease and/or disorder contexts include but are not limited to: apoptotic intestinal epithelial cells (in the context of colitis), apoptotic macrophages and stromal cells within atherosclerotic plaques (atherosclerosis), and apoptotic adipocytes (obesity).

In some embodiments, the plurality of metabolite compounds derived from an apoptotic cell is substantially pure. In some embodiments, the plurality of metabolite compounds is formulated in a single composition. In some embodiments, the plurality of metabolite compounds derived from an apoptotic cell are formulated in a pharmaceutically acceptable carrier. In some embodiments, the plurality of metabolite compounds are not purified from an apoptotic cell, but rather are provided from another source, as would be apparent to one of ordinary skill in the art upon a review of the instant disclosure, and formulated into a composition in accordance with the presently disclosed subject matter. In this case, an effective amount of the plurality of metabolite compounds is provided in the composition, such as amount corresponding to an amount purified from an apoptotic cell or a sample comprising an apoptotic cell. In some embodiments, the plurality of metabolite compounds comprises three or more metabolite compounds derived from an apoptotic cell. In some embodiments, the metabolite compound is selected from the group comprising spermidine, fructose 1,6-bisphosphate (FBP), dihydroxyacetone phosphate (DHAP), guanosine 5′-monophosphate (GMP), inosine 5′-monophosphate (IMP), and UDP-glucose and combinations thereof.

In some embodiments, the presently disclosed subject matter provides a composition comprising, consisting essentially of, or consisting of an effective amount of a plurality of metabolite compounds derived from an apoptotic cell; and a carrier. In some embodiments, the carrier is a pharmaceutically acceptable carrier.

In some embodiments, the plurality of metabolite compounds derived from an apoptotic cell is substantially pure. In some embodiments, the plurality of metabolite compounds is formulated in a single composition. In some embodiments, the plurality of metabolite compounds derived from an apoptotic cell are formulated in a pharmaceutically acceptable carrier. In some embodiments, the plurality of metabolite compounds are not purified from an apoptotic cell, but rather are provided from another source, as would be apparent to one of ordinary skill in the art upon a review of the instant disclosure, and formulated into a composition in accordance with the presently disclosed subject matter. In this case, an effective amount of the plurality of metabolite compounds is provided in the composition, such as amount corresponding to an amount purified from an apoptotic cell or a sample comprising an apoptotic cell. In some embodiments, the plurality of metabolite compounds comprises three or more metabolite compounds derived from an apoptotic cell. In some embodiments, the metabolite compound is selected from the group comprising spermidine, fructose 1,6-bisphosphate (FBP), dihydroxyacetone phosphate (DHAP), guanosine 5′-monophosphate (GMP), inosine 5′-monophosphate (IMP), and UDP-glucose and combinations thereof.

In some embodiments, the composition is for use in treating an inflammatory condition, for use in preparing a medicament for treating an inflammatory condition; for use in modulating gene expression in a cell; and/or for use in preparing a medicament for modulating gene expression in a cell. In some embodiments, the inflammatory condition is selected from the group comprising arthritis, transplantation rejection, colitis, peritonitis, and atherosclerosis. In some embodiments, the composition induces an anti-inflammatory response in a macrophage, a myeloid cell, a non-professional phagocyte, or any combination thereof. In some embodiments, the gene expression is involved in a biological process selected from the group consisting of inflammation, wound healing, proliferation, and development.

In some embodiments, the presently disclosed subject matter provides for the use of a pharmaceutical composition comprising, consisting essentially of, or consisting of an effective amount of a plurality of metabolite compounds derived from an apoptotic cell to treat an inflammatory condition in a subject in need thereof. In some embodiments, the presently disclosed subject matter provides for the use of an effective amount of a plurality of metabolite compounds derived from an apoptotic cell for the preparation of a medicament to treat an inflammatory condition in a subject in need thereof. In some embodiments, the presently disclosed subject matter provides a pharmaceutical composition comprising, consisting essentially of, or consisting of an effective amount of a plurality of metabolite compounds derived from an apoptotic cell to treat an inflammatory condition in a subject in need thereof.

The presently disclosed subject matter is also directed to methods of administering the compositions of the presently disclosed subject matter to a subject and to methods of contacting a cell with the compositions of the presently disclosed subject matter. In some embodiments, the cell is a cell in a subject.

Pharmaceutical compositions comprising the present composition comprising plurality of metabolite compounds derived from an apoptotic cell are administered to a subject in need thereof by any number of routes including, but not limited to, topical, oral, intravenous, intramuscular, intra-arterial, intramedullary, intrathecal, intraventricular, transdermal, subcutaneous, intraperitoneal, intranasal, enteral, topical, sublingual, or rectal approaches.

In accordance with one embodiment, a method for treating a subject in need of such treatment is provided. The method comprises administering a pharmaceutical composition comprising at least one composition of the presently disclosed subject matter to a subject in need thereof. Compositions provided by the methods of the presently disclosed subject matter can be administered with known compounds or other medications as well.

By way of representative, non-limiting example, the pharmaceutical compositions useful for practicing the presently disclosed subject matter may be administered to deliver a dose of a composition comprising a plurality of metabolite compounds derived from an apoptotic cell. Based on the instant disclosure and the level of skill in the art, dosage amounts and ranges can be established without undue experimentation using ordinary skill in the art.

The presently disclosed subject matter encompasses the preparation and use of pharmaceutical compositions comprising a composition comprising a plurality of metabolite compounds derived from an apoptotic cell useful for treatment of the diseases and disorders disclosed herein as an active ingredient. Such a pharmaceutical composition can consist of the active ingredient alone, in a form suitable for administration to a subject, or the pharmaceutical composition can comprise the active ingredient and one or more pharmaceutically acceptable carriers, one or more additional ingredients, or some combination of these. The active ingredient can be present in the pharmaceutical composition in the form of a physiologically acceptable ester or salt, such as in combination with a physiologically acceptable cation or anion, as is well known in the art.

As used herein, the term “physiologically acceptable” ester or salt means an ester or salt form of the active ingredient which is compatible with any other ingredients of the pharmaceutical composition, which is not deleterious to the subject to which the composition is to be administered.

The compositions of the presently disclosed subject matter can comprise at least one active ingredient, one or more acceptable carriers, and optionally other active ingredients or therapeutic agents.

Pharmaceutically acceptable carriers include physiologically tolerable or acceptable diluents, excipients, solvents, or adjuvants. The compositions are in some embodiments sterile and nonpyrogenic. Examples of suitable carriers include, but are not limited to, water, normal saline, dextrose, mannitol, lactose or other sugars, lecithin, albumin, sodium glutamate, cysteine hydrochloride, ethanol, polyols (propylene glycol, polyethylene glycol, glycerol, and the like), vegetable oils (such as olive oil), injectable organic esters such as ethyl oleate, ethoxylated isosteraryl alcohols, polyoxyethylene sorbitol and sorbitan esters, microcrystalline cellulose, aluminum methahydroxide, bentonite, kaolin, agar-agar and tragacanth, or mixtures of these substances, and the like.

The pharmaceutical compositions can also contain minor amounts of nontoxic auxiliary pharmaceutical substances or excipients and/or additives, such as wetting agents, emulsifying agents, pH buffering agents, antibacterial and antifungal agents (such as parabens, chlorobutanol, phenol, sorbic acid, and the like). Suitable additives include, but are not limited to, physiologically biocompatible buffers (e.g., tromethamine hydrochloride), additions (e.g., 0.01 to 10 mole percent) of chelants (such as, for example, DTPA or DTPA-bisamide) or calcium chelate complexes (as for example calcium DTPA or CaNaDTPA-bisamide), or, optionally, additions (e.g., 1 to 50 mole percent) of calcium or sodium salts (for example, calcium chloride, calcium ascorbate, calcium gluconate or calcium lactate). If desired, absorption enhancing or delaying agents (such as liposomes, aluminum monostearate, or gelatin) can be used. The compositions can be prepared in conventional forms, either as liquid solutions or suspensions, solid forms suitable for solution or suspension in liquid prior to injection, or as emulsions. Pharmaceutical compositions according to the presently disclosed subject matter can be prepared in a manner fully within the skill of the art.

The compositions of the presently disclosed subject matter or pharmaceutical compositions comprising these compositions can be administered so that the compositions may have a physiological effect. Administration can occur enterally or parenterally; for example, orally, rectally, intracisternally, intravaginally, intraperitoneally, locally (e.g., with powders, ointments or drops), or as a buccal or nasal spray or aerosol. Parenteral administration is an approach. Particular parenteral administration methods include intravascular administration (e.g., intravenous bolus injection, intravenous infusion, intra-arterial bolus injection, intra-arterial infusion and catheter instillation into the vasculature), peri- and intra-target tissue injection, subcutaneous injection or deposition including subcutaneous infusion (such as by osmotic pumps), intramuscular injection, and direct application to the target area, e.g., intratumoral injection, for example by a catheter or other placement device.

Where the administration of the composition is by injection or direct application, the injection or direct application can be in a single dose or in multiple doses. Where the administration of the compound is by infusion, the infusion can be a single sustained dose over a prolonged period of time or multiple infusions.

The formulations of the pharmaceutical compositions described herein can be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

It will be understood by the skilled artisan that such pharmaceutical compositions are generally suitable for administration to animals of all sorts. Subjects to which administration of the pharmaceutical compositions of the presently disclosed subject matter is contemplated include, but are not limited to, humans and other primates, mammals including commercially and/or socially relevant mammals such as cattle, pigs, horses, sheep, cats, and dogs, birds including commercially and/or socially relevant birds such as chickens, ducks, geese, parrots, and turkeys.

A pharmaceutical composition of the presently disclosed subject matter can be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the presently disclosed subject matter will vary, depending upon the identity, size, and condition of the subject treated and further depending upon the route by which the composition is to be administered. By way of example, the composition can comprise between 0.1% and 100% (w/w) active ingredient.

In addition to the active ingredient, a pharmaceutical composition of the presently disclosed subject matter can further comprise one or more additional pharmaceutically active agents. Particularly provided additional pharmaceutically active agents include chemotherapeutic agents, antibody drug conjugates, and liposomes or vesicles carrying specific metabolites.

Controlled- or sustained-release formulations of a pharmaceutical composition of the presently disclosed subject matter can be made using conventional technology.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the presently disclosed subject matter are known in the art and described, for example in Gennaro (1990) Remington’s Pharmaceutical Sciences. 18th ed., Mack Pub. Co., Easton, Pennsylvania, United States of America and/or Gennaro (ed.) (2003) Remington: The Science and Practice of Pharmacy, 20th edition Lippincott, Williams & Wilkins, Philadelphia, Pennsylvania, United States of America, each of which is incorporated herein by reference.

The compositions may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type of cancer being diagnosed, the type and severity of the condition or disease being treated, the type and age of the animal, etc.

Other approaches include but are not limited to nanosizing the composition comprising a plurality of metabolite compounds derived from an apoptotic cell to be delivered as a nanoparticle intravenously, intraperitoneal injection, or implanted beads with time release of a plurality of metabolite compounds derived from an apoptotic cell. In some embodiments, the composition comprising a plurality of metabolite compounds derived from an apoptotic cell is adapted for administration for the treatment of a human patient by injecting dose of a plurality of metabolite compounds derived from an apoptotic cell by intravenous administration, intrathecal injection, peritoneal injection, or direct injection into the tumor or surround tumor site. In some embodiments, the composition comprising a plurality of metabolite compounds derived from an apoptotic cell is adapted for administration for the treatment of an animal patient (dogs, cats, cows, horses, and pigs by injecting dose of a plurality of metabolite compounds derived from an apoptotic cell by intravenous administration, peritoneal injection, or direct injection into the tumor or surround tumor site.

Suitable preparations include injectables, either as liquid solutions or suspensions, however, solid forms suitable for solution in, suspension in, liquid prior to injection, may also be prepared. The preparation may also be emulsified, or the compositions encapsulated in liposomes. The active ingredients are often mixed with excipients which are pharmaceutically acceptable and compatible with the active ingredient. Suitable excipients are, for example, water saline, dextrose, glycerol, ethanol, or the like and combinations thereof. In addition, if desired, the preparation may also include minor amounts of auxiliary substances such as wetting or emulsifying agents, pH buffering agents, and/or adjuvants.

The presently disclosed subject matter also includes a kit comprising the composition of the presently disclosed subject matter and an instructional material which describes administering the composition to a cell or a tissue of a subject. In some embodiments, this kit comprises a (in some embodiments sterile) solvent suitable for dissolving or suspending the composition of the presently disclosed subject matter prior to administering the compound to the subject and/or a device suitable for administering the composition such as a syringe, injector, or the like or other device as would be apparent to one of ordinary skill in the art upon a review of the instant disclosure.

As used herein, an “instructional material” includes a publication, a recording, a diagram, or any other medium of expression which can be used to communicate the usefulness of the composition of the presently disclosed subject matter in the kit for effecting alleviation of the various diseases or disorders recited herein. Optionally, or alternately, the instructional material may describe one or more methods of using the compositions for diagnostic or identification purposes or of alleviation the diseases or disorders in a cell or a tissue of a warm-blooded vertebrate, e.g. a mammal. The instructional material of the kit of the presently disclosed subject matter can, for example, be affixed to a container which contains a composition of the presently disclosed subject matter or be shipped together with a container which contains the composition. Alternatively, the instructional material can be shipped separately from the container with the intention that the instructional material and the composition be used cooperatively by the recipient.

In accordance with the presently disclosed subject matter, as described above or as discussed in the EXAMPLES below, there can be employed conventional chemical, cellular, histochemical, biochemical, molecular biology, microbiology, recombinant DNA, and clinical techniques which are known to those of skill in the art. Such techniques are explained fully in the literature. See for example, Sambrook et al. (1989) Molecular Cloning: A Laboratory Manual, Cold Spring Harbor Laboratory Publications, Cold Spring Harbor, New York, United States of America; Glover (1985) DNA Cloning: A Practical Approach. Oxford Press, Oxford; Gait (1984) Oligonucleotide Synthesis: A Practical Approach, IRL Press, Oxford, England; Harlow & Lane, 1988, Antibodies, A Laboratory Manual, Cold Spring Harbor Publications, New York; Roe et al. (1996) DNA Isolation and Sequencing: Essential Techniques, John Wiley, New York, New York, United States of America; and Ausubel et al. (1995) Current Protocols in Molecular Biology, Greene Publishing.

### III. EXAMPLES

The following EXAMPLES provide illustrative embodiments. In light of the present disclosure and the general level of skill in the art, those of skill will appreciate that the following EXAMPLES are intended to be exemplary only and that numerous changes, modifications, and alterations can be employed without departing from the scope of the presently disclosed subject matter.

Without further description, it is believed that one of ordinary skill in the art can, using the preceding description and the illustrative EXAMPLES, make and utilize the methods and compositions of the presently disclosed subject matter and practice the methods of the presently disclosed subject matter. The EXAMPLES therefore particularly point out embodiments of the presently disclosed subject matter and are not to be construed as limiting in any way the remainder of the disclosure.

### Materials and Methods Employed in Examples

Reagents. Trovafloxacin, spironolactone, dexamethasone, spermidine, fructose 1,6-bisphosphate, dihydroxyacetone phosphate, inosine 5′-monophosphate, and guanosine 5′-monophosphate were obtained from Sigma. UDP-glucose was obtained from Abcam and Annexin V-Pacific Blue was from BioLegend. 7AAD, TO-PRO-3, anti-CD11b-PE (clone M1/70), anti-CD11c-PE (clone N418), and anti-CD16/CD32 (clone 93) were obtained from Invitrogen. Antibodies specific for mouse CD95 were obtained from BD. Human anti-Fas (clone CH11) was obtained from Millipore. Other reagents were obtained as follows: ABT-737 (abcam), TRAIL (Sigma), and zVAD-FMK (Enzo).

Mice. C57BL/10 and C57BL/6J wild-type mice were acquired from Jackson Laboratories. Panxl^{ƒl/ƒl} and Panx1^{-/-} mice have been described previously¹¹. To generate mice with deletion of Panx1 in thymocytes, Panxl^{ƒl/ƒl} mice were crossed to Cd4-Cre mice (Taconic). KRN TCR transgenic mice were a gift from Dr. Diane Mathis at the Harvard Medical School, and were bred to NOD mice (Jackson Laboratories) to obtain the K/BxN mice, which develop progressive spontaneous arthritis²⁹. K/BxN serum was collected from 9-week old K/BxN mice by cardiac puncture. Animal procedures were approved and performed according to the Institutional Animal Care and Use Committee (IACUC) at the University of Virginia.

Apoptosis induction. Wild type Jurkat E6.1 (ATCC) or dominant negative Pannexinl-expressing (Panxl-DN)¹⁰ cells were resuspended in RPMI-1640 containing 1% BSA, 1% PSQ, and 10 mM HEPES and treated with 250 ng ml⁻¹ anti-Fas (clone CH11), 10 µM ABT-737, or exposed to 150 mJ cm⁻² ultraviolet C irradiation for 1-2 min (Stratalinker). Jurkat cells were incubated for 4 hours after apoptosis induction. For apoptosis induction in the presence of Panx1 inhibitors, Jurkat cells were treated with spironolactone (50 µM) or trovafloxacin (25 µM) in RPMI containing 1% BSA and 1% PSQ.

Primary thymocytes isolated from 4 to 6-week old wild-type or Panx1 ^{-/-} mice were treated with 5 µg ml⁻¹ anti-Fas (clone Jo2), that was subsequently crosslinked with 2 µg ml⁻¹ protein G. Primary thymocytes were incubated for 1.5 hours after apoptosis induction.

B6^{Nlrp1b}C1^{-/-}C11^{-/-} were a gift from Dr. Mohamed Lamkanfi’s lab (VIB/LJGent, Belgium). BMDMs were generated by culturing mouse bone marrow cells in RPMI media conditioned with 10% dialyzed serum and 1% Pen-strep. The medium was supplemented with 20 ng/ml of purified mouse M-CSF. Cells were incubated in a humidified atmosphere containing 5% CO₂ for 6 days. WT B6 or B6^{Nlrp1b+}c1^{-/-}C11^{-/-} BMDMs were seeded in 6-well plates and, the next day, either left untreated or stimulated with 500 ng/mL with anthrax PA (500 ng/mL, Quadratech) and LF (250 ng/mL, Quadratech). Supernatants from either untreated or treated BMDMs was collected. Cellular debris was removed via centrifugation step and the clarified supernatant was used for metabolic profiling.

A549 cells were treated with 10 µM ABT-737 or exposed 600mJ cm⁻² and incubated for twenty-four hours. HCT-116 cells were treated with 10 µM ABT-737 or 100 ng ml⁻¹ TRAIL and incubated for 24 hours. All cells were pre-treated for 10 min with 50 uM zVAD prior to apoptosis induction in indicated experiments. All cells were incubated at 37° C. with 5% CO₂ for indicated times.

Metabolite detection. Spermidine detection was measured using a colorimetric kit (Cloud-Clone Corp.) via manufacturers’ protocol. Briefly, supernatants taken from cells under specified conditions were centrifuged at 1000 x g for twenty minutes. All reagents were brought to room temperature prior to use. 50 µl of sample were added to each well followed by equal volume of Detection Reagent A and the plate was mixed. Samples were incubated, covered, for one hour at 37° C. Wells were washed with 1x Wash solution three times before addition of Detection Reagent B, after which samples were incubated for another thirty minutes at 37° C. Samples were washed again five times. 90 µl of substrate solution was added to each well and incubated for 10 minutes at 37° C. after which 50 µl of stop solution was added, the plate was mixed and immediately measure at 450 nm on a plate reader (Flex station 3). Analysis was performed by back calculation to the standard curve, background subtraction and normalization to live cell controls.

ATP was measure using a luciferase-based kit (Promega) via manufactures’ protocol. All reagents were equilibrated to room temperature before use. Briefly, supernatants taken from cells under specified conditions, were immediately moved to ice, and centrifuged at 500 x g for 5 minutes. Samples were placed back on ice and 50 ul of samples and 50 ul of luciferase reagent were mixed in a 96 well opaque plate. Luminescence was immediately measure on the Flex Station 3. Analysis was performed by back calculation to the standard curve, background subtraction and normalization to live cell controls.

Glycerol-3-phosphate and creatine were measured based on manufacturers’ protocols (Abcam). Briefly, supernatants were taken from specified culture conditions and spun at 500 x g. 50 µl of supernatant was added to a 96 well plate. Detection reagents were prepared as indicated in protocol and added to respective wells. Samples were incubated for 40 minutes or 1 hr. for glycerol-3-phosphate and creatine, respectively. OD at 450 nm or fluorescence at Ex/Em 535/587 was measured for glycerol-3-phosphate and creatine, respectively.

Flow cytometry of apoptosis and Panx1 activation. Apoptotic cells were stained with Annexin V-Pacific Blue, 7AAD, and TO-PRO-3 for 15 minutes at room temperature in the Annexin V binding buffer (140 mM NaCl, 2.5 µM CaCl, 10 mM HEPES) and subjected to flow cytometry on Attune NxT (Invitrogen). Data were analyzed using FlowJo V10 Software.

Metabolomics analysis of apoptotic supernatant and cell pellet. Sample extraction, processing, compound identification, curation and metabolomic analyses were carried out at Metabolon Inc. (Durham, NC) and Human Metabolome Technologies America (HMT) (Boston, MA) ³⁰. Briefly, supernatants were separated from cell pellets via sequential centrifugation and frozen before shipment for metabolomic analysis. For HMT; supernatant samples were spiked with 10 ul of water with internal standards, then filtered through a 5-kDa cut-off filter to remove macromolecules and small vesicles. Cationic compounds were diluted and measured using positive ion mode ESI via CE-TOFMS. Anionic compounds were measures in the positive or negative ion mode ESI using CE-MS/MS. Samples were diluted to improve the CE-QqQMS analysis. Peak identification and metabolite quantification were determined using migration time, mass to charge ratio, and the peak area normalized to the internal standard and standard curves. Concentrations reported are on a per million cell basis which was derived by back calculations on the cell number that was used in the experimental set-up.

For untargeted metabolomics analysis through Metabolon, recovery standards were added to samples in order to monitor QC of the analysis. Samples were methanol precipitated under shaking for 2 minutes. After, samples were placed on the TurboVap to remove organic solvent and the samples were stored O/N under nitrogen. Samples were analyzed under 4 different conditions; two for analysis by two separate reverse phase (RP)/UPLC-MS/MS methods with positive ion mode ESI, one for analysis by RP/UPLC-MS/MS with negative ion mode ESI, and one for analysis by HILIC/UPLC-MS/MS with negative ion mode ESI. Using a library based on authenticated standards that contains the retention time/index (RI), mass to charge ratio (m/z), and chromatographic data (including MS/MS spectral data) on all molecules in the library (Metabolon), the metabolite identification could be performed with reverse scores between the experimental data and authenticated standards. While there may be similarities based on one of these factors, the use of all three data points can be used to identify biochemicals. R code used for heatmap generation and volcano plots is available upon request.

Metabolite flux experiments with ¹³C-arginine labeling. Cells were re-suspended in arginine free RPMI media containing 10% dialyzed serum, supplemented with 1 mM 13C6 L-Arginine HCl (Thermo Fischer Scientific). Cells were either exposed to UV or left untreated. This step was performed within a minute of adding the media containing ¹³C-arginine to cells. The cells were then incubated at 37° C. Samples were collected every hour to trace the incorporation of the label from arginine into the polyamine pathway for both UV exposed and live cells. Where indicated, the cells were pre-treated with zVAD-FMK to inhibit caspases.

Metabolite extraction from the pellet or supernatant was performed by adding 300 ul of 6%TCA to a pellet of 4 million cells on ice. The samples were then vortexed thoroughly at 4° C., followed by centrifugation to remove cell debris. 100ul of the supernatant and 900 ul of Na2Co3 (0.1 M, pH 9.3) were mixed, followed by the addition of 25 ul of isobutyl chloroformate. The mixture was incubated at 37° C. for 30 minutes and then centrifuged for 10 minutes at 20000 g. 800 ul of the supernatant was transferred to a fresh tube, followed by the addition of 1000 µl of diethyl ether and vortexing. The mixture was allowed to sit at RT for 10 minutes for phase separation after which, 900 µl sample was collected in a fresh Eppendorf tube. The samples were dried via Speedvac. For LC-MS runs, 150 ul of 1:1 mixture of 0.2% acetic acid in water and 0.2% of acetic acid in acetonitrile was added to the dried sample.

RNA-sequencing. LR73 cells (ATCC) were plated at (100x10³) per well in 24-well tissue culture plates and cultured for 16 hours at 37° C. with 5% CO₂. The cells were then rinsed with phosphate buffered saline (PBS), and fresh supernatants taken from live Jurkat, apoptotic Jurkat (UV), or Panxl-DN apoptotic Jurkat (UV) cells were added for 4 hours (as described above). Total RNA was harvested using the Nucleospin RNA kit (Macherey-Nagal) and an mRNA library was constructed with Illumina TruSeq platform. Transcriptome sequencing using an Illumina NextSeq 500 cartridge was then performed on samples from four independent experiments. RNAseq data was analyzed using Rv1.0.136 and the R package DeSeq2 for differential gene expression, graphical representation, and statistical analysis. R code used for bioinformatic analysis and heatmap generation is available upon request.

Quantitative RT-PCR analysis. RNA was extracted from cells treated with different live or apoptotic supernatants. Where indicated, supernatants were filtered through a 3 kDa filter as suggested by manufacturers’ protocol. Briefly, supernatants were separated from cells and large vesicles via sequential centrifugations. Supernatants were then added to 3 kDA filters (Millipore) and centrifuged for one hour at 3000 x g prior to adding supernatant to live LR73 cells. Nucleospin RNA kit (Macherey-Nagel) was used for RNA extraction and cDNA was synthesized using QuantiTect Reverse Transcription Kit (Qiagen). Gene expression of indicated genes was performed using Taqman probes (Applied Biosystems) and the StepOnePlus Real Time PCR System (Applied Biosystems).

In vivo thymocyte death induction. Six- to eight-week old Panxl^{ƒl/ƒl} or Panxl^{ƒl/ƒl}Cd4-Cre mice were injected intraperitoneally with dexamethasone (250 µg). Thymus was harvested 6 hours post-injection and single cell suspension was prepared using 70-µm strainers (Fisher). An aliquot of digested tissue was taken to measure the extent of thymocyte cell death and Pannexin1 activation using Annexin V-Pacific Blue, 7AAD, and TO-PRO-3, as described above. Samples were acquired on Attune NxT (Invitrogen) and analyzed using FlowJo v10 Software.

Thymic myeloid cell isolation and gene expression. Six- to eight-week old Panxl^{ƒl/ƒl} or Panxl^{ƒl/ƒl}Cd4-Cre mice were injected with dexamethasone and single cell suspensions of thymus were prepared as described above. Following isolation, cells were incubated with anti-CD16/CD32 (Fc-Block, Invitrogen) for 20 minutes at 4° C. Cells were then stained with anti-CD3-PE and run through a MACS kit using anti-PE microbeads to ‘de-bulk’ the cell suspension and remove a majority of thymocytes. Cell flow through (CD3neg population) was collected and then stained with anti-CD11b-PE and anti-CD11c-PE antibodies 30 minutes at 4° C. Stained cells were purified using the anti-PE MicroBeads MACS kit (Miltenyi Biotec), following manufacturers protocol. Sample aliquots were run on the Attune NxT (Invitrogen) and analyzed using FlowJo v10 Software. Total RNA from purified cells was isolated Nucleospin RNA kit (Macherey-Nagel) for cDNA synthesis and qRT-PCR, as described above.

MeMix preparation and in vivo treatment. The metabolite mixture MeMix⁶ was composed of these six metabolites: spermidine, fructose 1,6-bisphosphate (FBP), dihydroxyacetone phosphate (DHAP), guanosine 5′-monophosphate (GMP), inosine 5′-monophosphate (IMP), and UDP-glucose. MeMix³ was composed of spermidine, GMP and IMP. Concentrations of metabolites used for in vitro LR73 phagocyte treatment were as follows (based on targeted metabolomics): IMP (3.3 µM), DHAP (36 µM), FBP (0.5 µM), GMP (2.1 µM), UDP-Glucose (2 µM), Spermidine (0.3 µM). Concentration of metabolites used for in vivo mice treatment were as follows: IMP (100 mg/kg), DHAP (50 mg/kg), FBP (500 mg/kg), GMP (100 mg/kg), UDP-Glucose (100 mg/kg), Spermidine (100 mg/kg).

K/BxN induced arthritis. C57BL/6J mice were given intraperitoneal injections of 150 µl of serum from K/BxN mice on day 0 and paw swelling was measured at indicated time points using a caliper (Fisher). Measurements are presented as percent change from day 0. On day 1, mice were randomly assigned into three groups and given daily intraperitoneal injections of either MeMix(^{3or6)} or vehicle through day 5. In separate experiments, mice on day 1 were randomly assigned and given daily injections of either live or apoptotic supernatants through day 5. Clinical scores were assigned for each paw as follows: 0 - no paw swelling or redness observed, 1 - redness of the paw or a single digit swollen, normal V shape of the hind foot (the foot at the base of the toes is wider than the heel and ankle) 2 - two or more digits swollen or visible swelling of the paw, U shape of the hind foot (the ankle and the midfoot are equal in thickness), 3 - reversal of the V shape of the hind foot into an hourglass shape (the foot is wider at the heel than at the base of the toes). A combined clinical score of all paws is presented. Paw measurements and clinical score assignments were performed by an investigator blinded to the treatment groups.

Lung Transplant Rejection Model. Orthotopic left lung transplantation was carried out according to previous reports. To study the alteration of allo-immune response by a minor antigen-mismatched combination, C57BL/10 donor and C57BL/6 recipients were used. The recipient mice were administrated with MeMix³ or vehicle intraperitoneally on post-operative Day 1 and Day 3. On Day 7, the recipient mice were sacrificed and left lung allografts were harvest and processed for histology.

Histology. Lungs were fixed in formalin, sectioned, and stained with hematoxylin and eosin (H&E). The acute rejections were graded according to the International Society for Heart and Lung Transplantation (ISHLT) A Grade criteria by a lung pathologist who is blinded to the experimental settings²⁸. For arthritis mice were euthanized at day 8 of K/BxN serum induced arthritis and the hind paws were fixed in 10% formalin (Fisher). Decalcification, sectioning, paraffin embedding, hematoxylin and eosin (H&E) staining and Safranin O staining was performed by HistoTox Labs (Boulder, CO). Images of ankle sections were taken on an EVOS FL Auto (Fisher) and analyzed using the accompanying software. Histology scoring was performed by an investigator blinded to the mouse treatment. For inflammation and cartilage erosion scoring, the following criteria were used - 0, none; 1, mild; 2, moderate; 3, severe. For bone erosion scoring, the following criteria were used: 0, no bone erosions observed; 1, mild cortical bone erosion; 2, severe cortical bone erosion without the loss of bone integrity; 3, severe cortical bone erosion with the loss of cortical bone integrity and trabecular bone erosion.

Statistical analysis. Statistical significance was determined using GraphPad Prism 7, using unpaired Student’s two-tailed t-test (paired and unpaired), one-way ANOVA, or two-way ANOVA according to test requirements. Grubbs’ Outlier Test was used to determine outliers, which were excluded from final analysis. A p value of <0.05 (indicated by one asterisk), <0.01 (indicated by two asterisks), <0.001 (indicated by three asterisks), or <0.0001 (indicated by four asterisks) were considered significant.

### Example 1

### Profiling the Metabolite Secretome of Apoptotic Cells

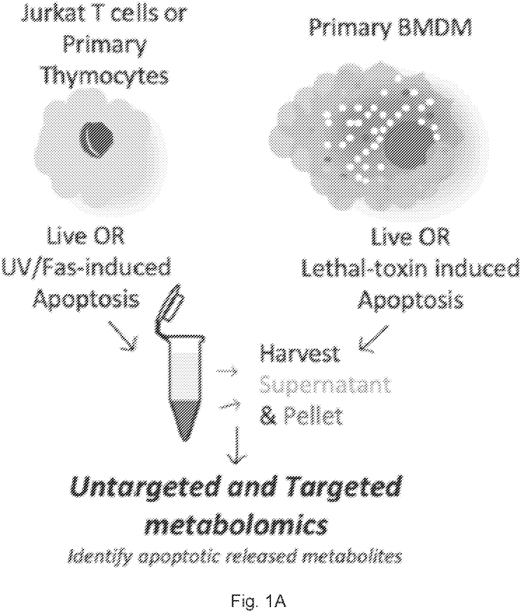

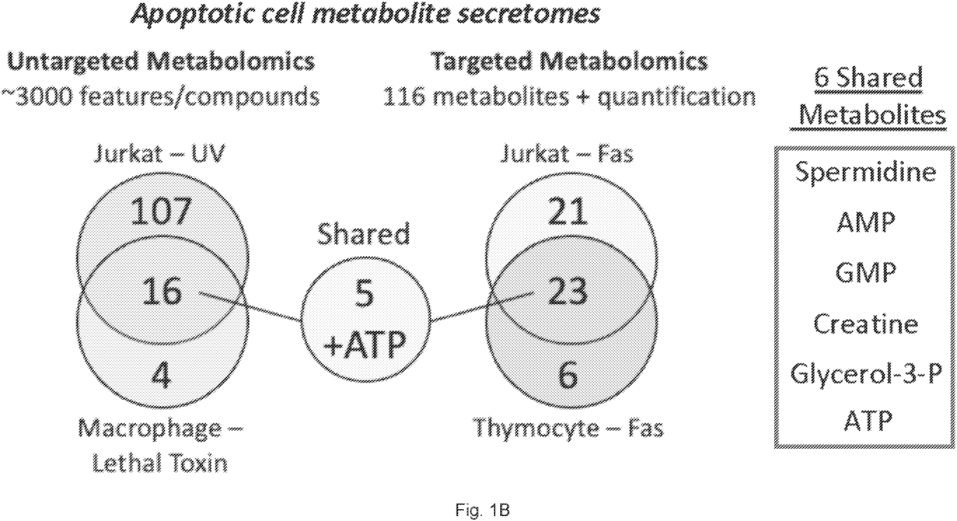

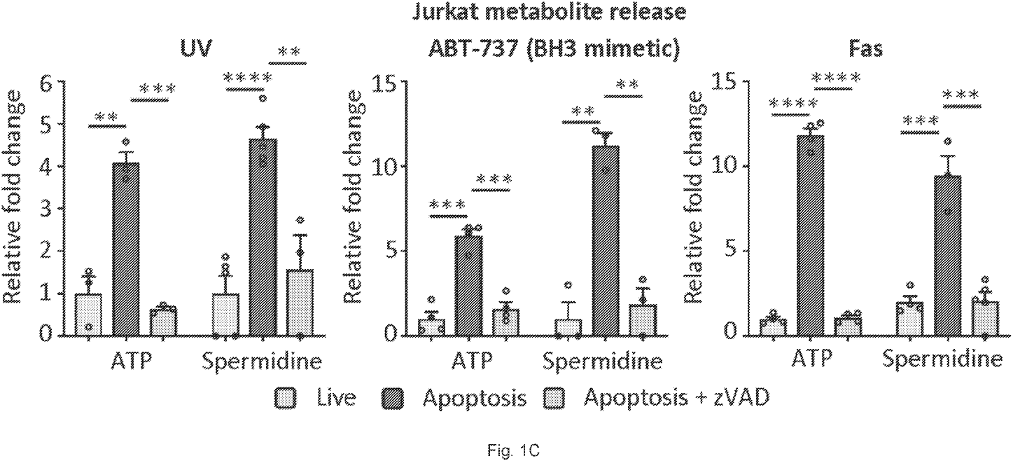

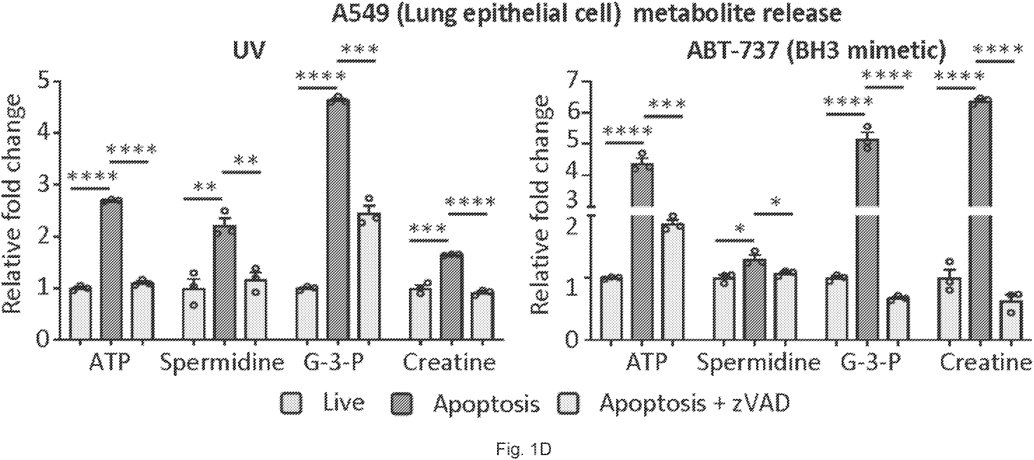

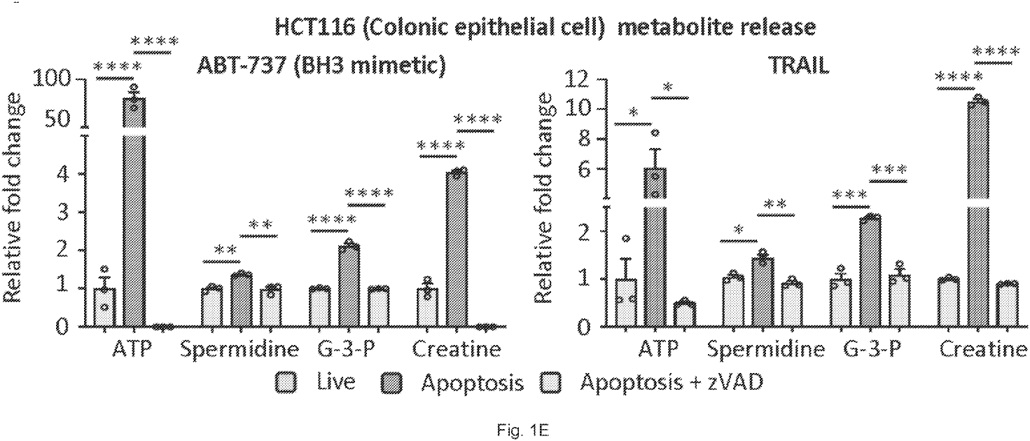

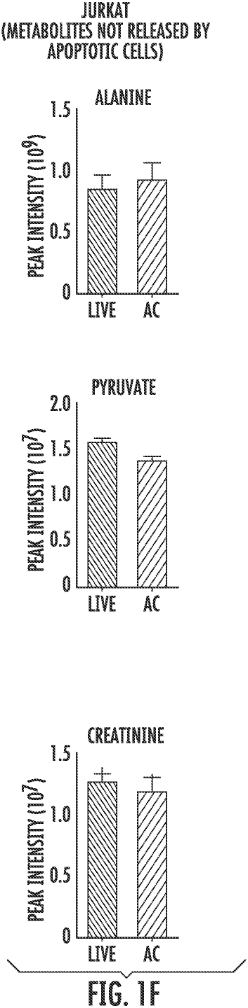

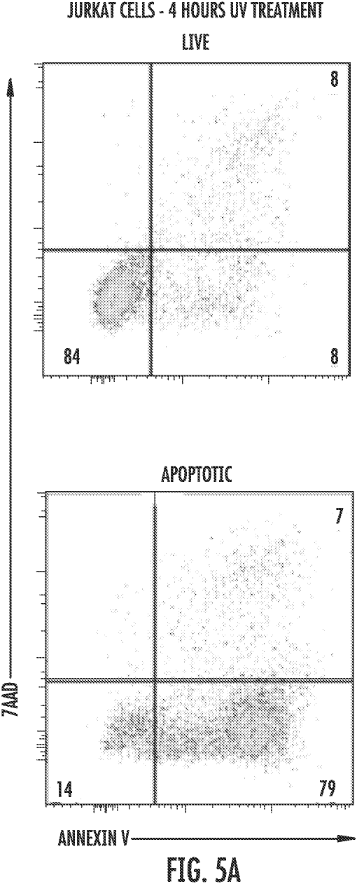

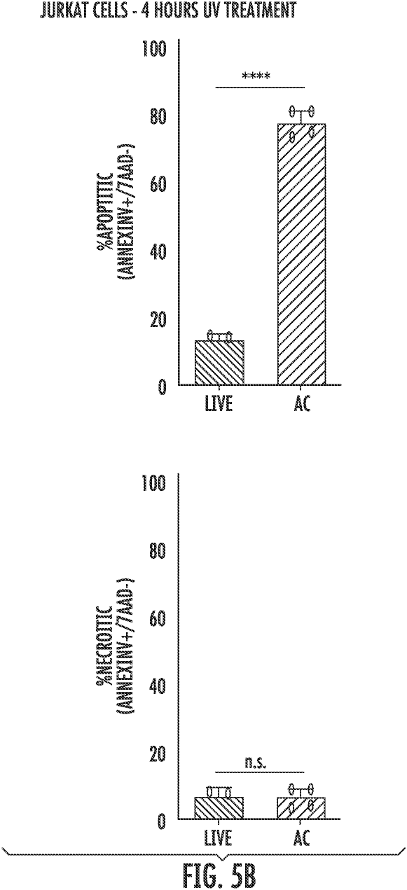

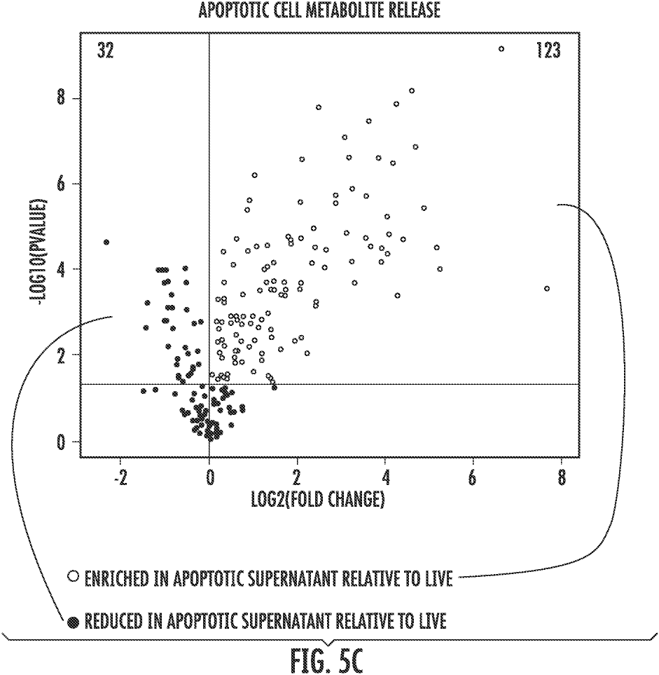

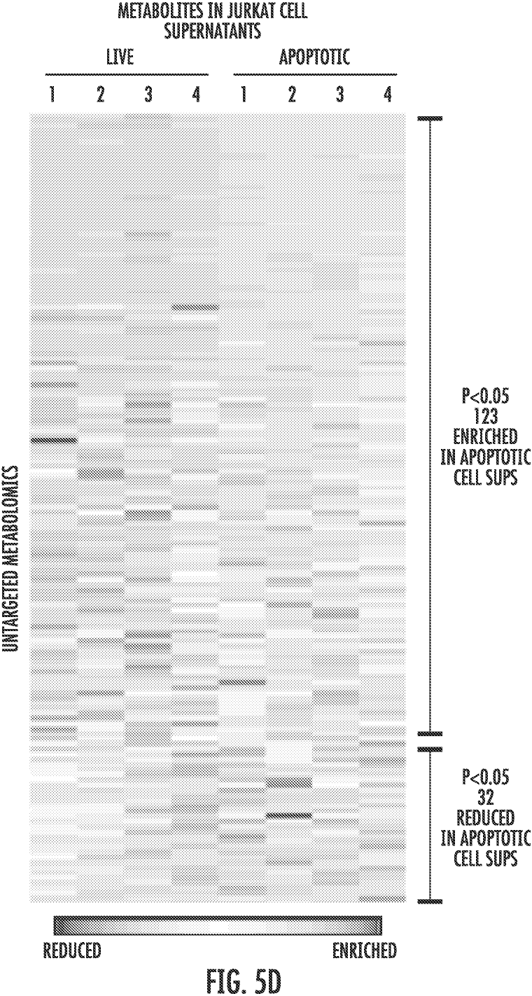

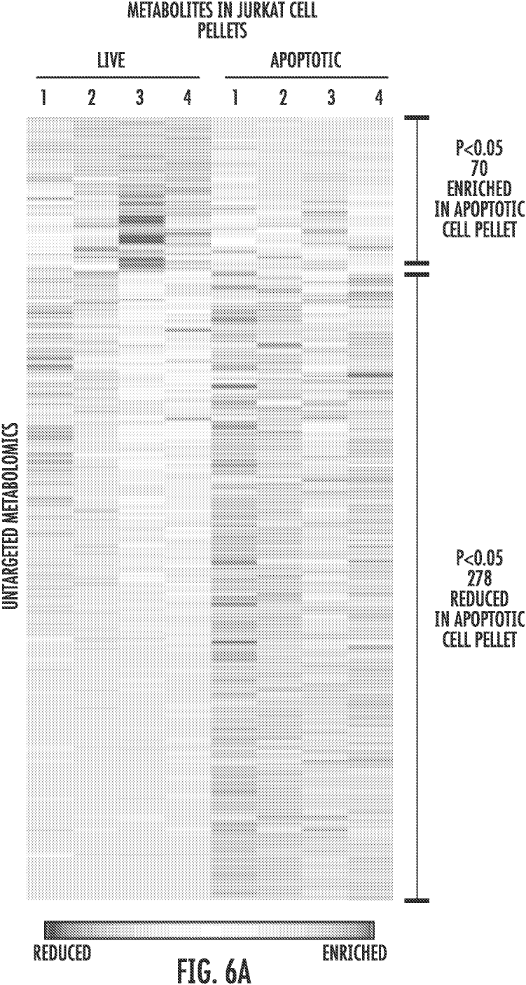

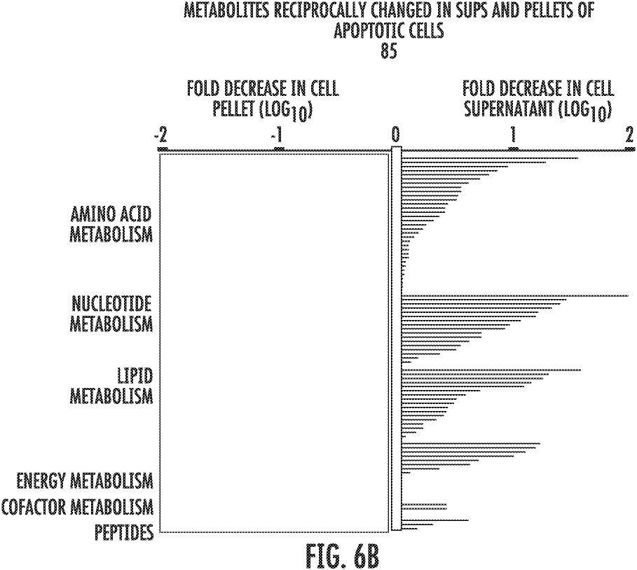

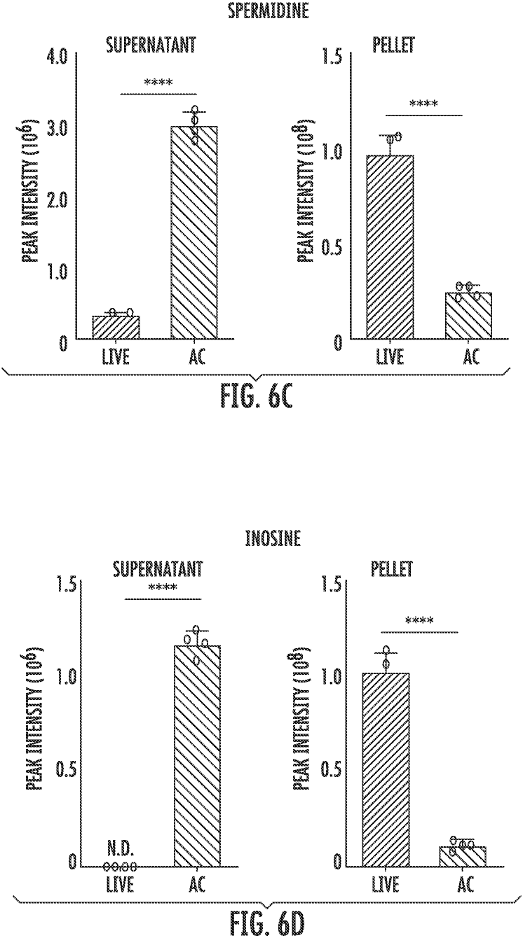

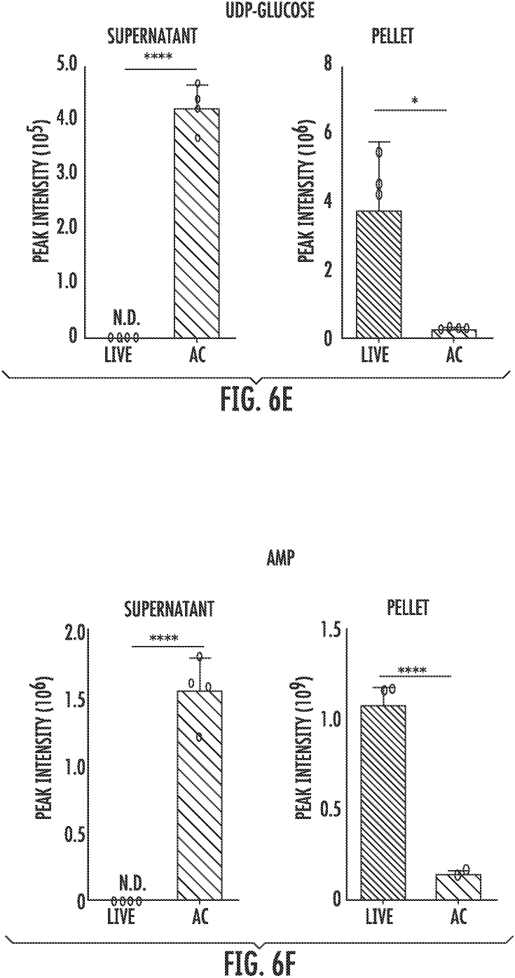

To profile the metabolite secretome of apoptotic cells, human Jurkat T cells, primary murine thymocytes, or primary bone-marrow derived macrophages (BMDM) were used, all of which can undergo inducible, caspase-dependent apoptosis (UV treatment, anti-Fas antibody crosslinking, or anthrax lethal toxin-induced apoptosis)^{8,9}(FIG. 1A). As untargeted metabolomics require large numbers of cells, we optimized the parameters using Jurkat cells (e.g. cell density, culture volume, duration after apoptosis), such that about 80% of the cells were apoptotic, while maintaining cell membrane integrity (Annexin V⁺7AAD⁻) (FIGS. 5A and 5B). Supernatants and cell pellets from apoptotic and live cell controls were subjected to untargeted metabolomic profiling against a library of greater than 3000 biochemical features/compounds. Supernatants of apoptotic Jurkat cells (UV) showed an enrichment of 123 metabolites (FIG. 1B, FIGS. 5C and 5D, Table 1), and 85 of these 123 were reciprocally reduced in the apoptotic cell pellets (FIGS. 6A-6F, Table 2).

In untargeted metabolomics of supernatants from macrophages undergoing apoptosis (via anthrax lethal toxin⁹), we detected fewer metabolites (20, versus 123 in Jurkat cells), perhaps due to differences in cell types, modality of death and/or quantities released (i.e. detection limits). Strikingly, 16 of the 20 metabolites (80%) were shared with apoptotic Jurkat cells (FIG. 1B).

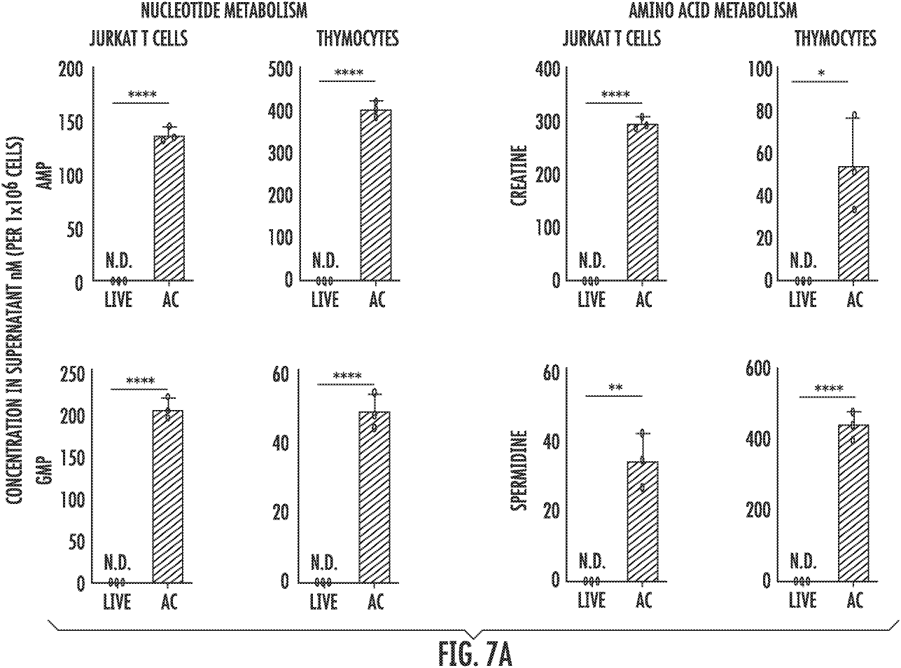

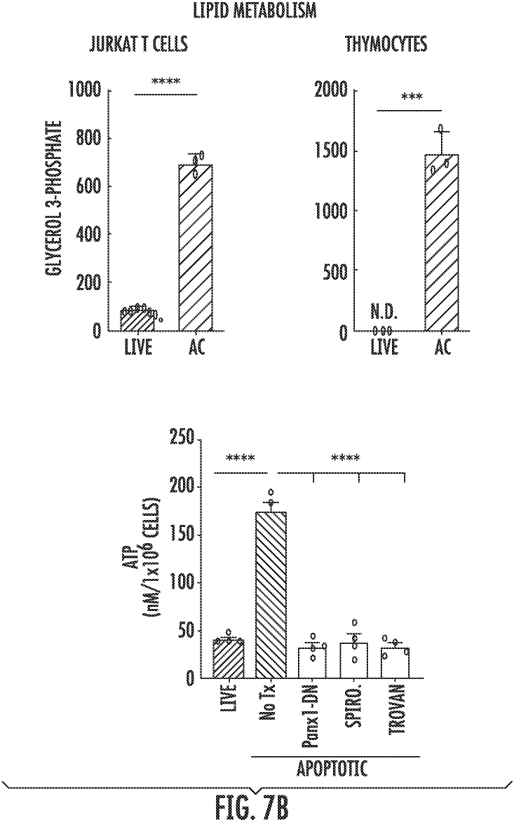

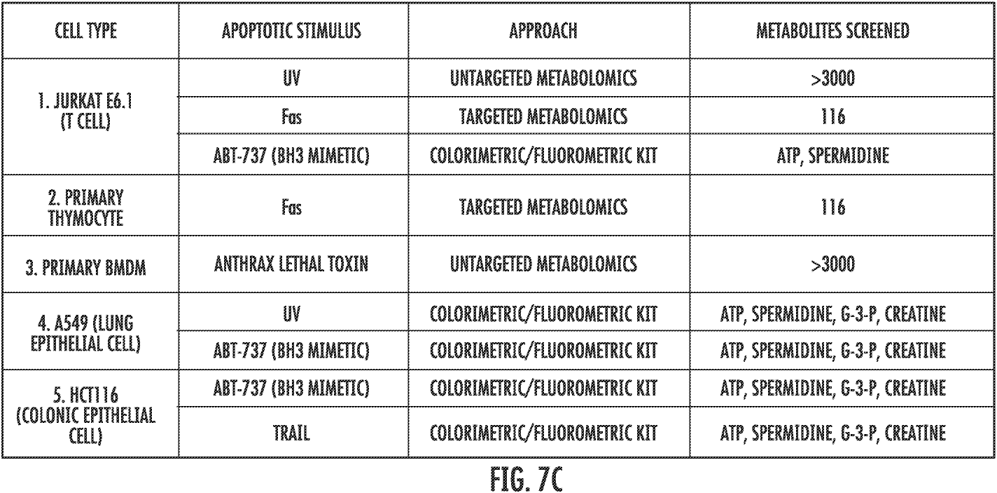

For further validation and quantitation, we performed “targeted metabolomics” analyzing 116 specific metabolites (see methods) on supernatants from Jurkat cells and primary murine thymocytes after Fas-crosslinking (“extrinsic” cue for apoptosis) (Table 3). This targeted panel included 43 of the metabolites released from apoptotic Jurkat cells (identified above), and included a 5 kDa filtering step (to exclude proteins, and extracellular vesicles). This targeted analysis showed an enrichment of many metabolites seen with UV-induced apoptosis (FIG. 1B). Further, metabolites released from apoptotic primary thymocytes overlapped with apoptotic Jurkat cells (FIG. 1B). Comparing metabolites enriched/released in the apoptotic supernatant of Jurkat cells, thymocytes, and macrophages (after Fas, UV, or toxin-mediated apoptosis) identified five ‘conserved’ metabolites: AMP, GMP, creatine, spermidine, and glycerol 3-phosphate (FIG. 1B, FIG. 7A). ATP represents the 6^{th} shared metabolite (via luciferase assay, FIG. 7B), although ATP was not profiled in the metabolomics.

To test other cell types and additional apoptotic modalities, we analyzed the release of four ‘conserved metabolites’ via analytical kits. Jurkat cells, A549 lung epithelial cells, and HCT116 colonic epithelial cells were induced to undergo death via different apoptotic cues, such as UV, BH3-mimetic ABT-737 (which directly induces mitochondrial outer membrane permeabilization), and/or TRAIL (extrinsic mode of apoptosis) (FIGS. 1C-1E). We could readily detect apoptosis-dependent release of the tested metabolites, and attenuation by pan-caspase inhibitor zVAD (FIGS. 1C-1E, FIG. 7C). The metabolites detected were not due to simple emptying of cellular contents during apoptosis, as many metabolites at high intracellular concentrations were not released (FIG. 1F). These data reveal apoptotic cells as a novel ‘natural source’ of many metabolites with biological functions.

### Example 2

### Testing of Specific Channels Opening During Apoptosis

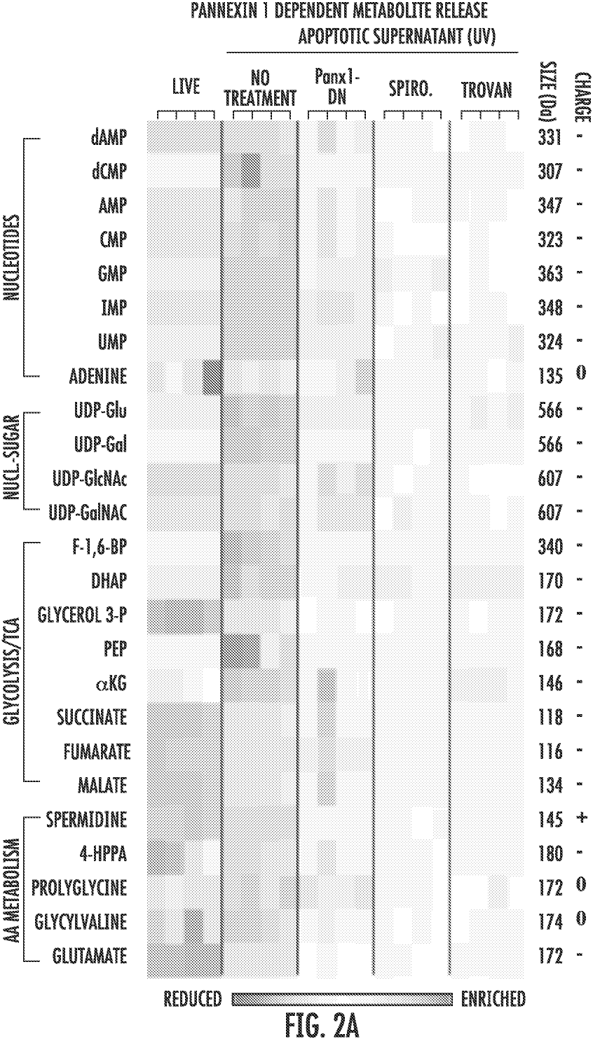

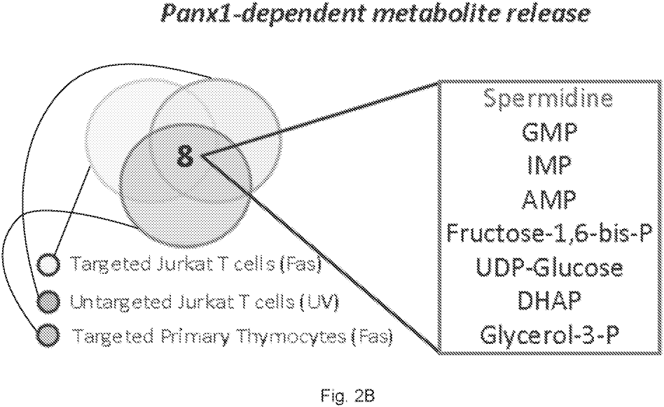

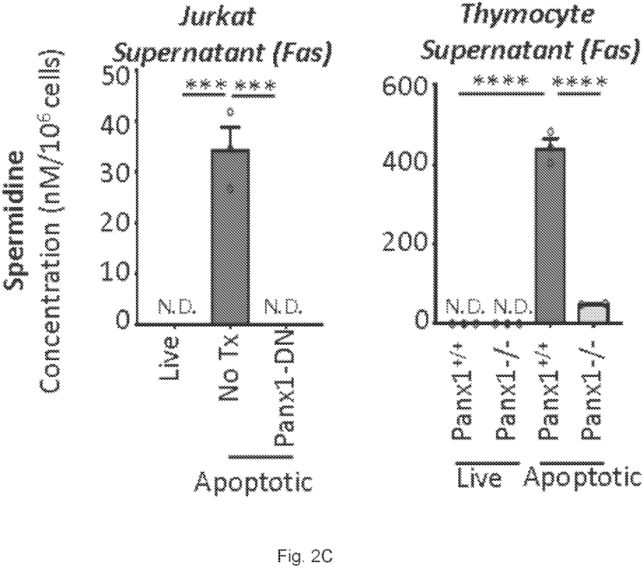

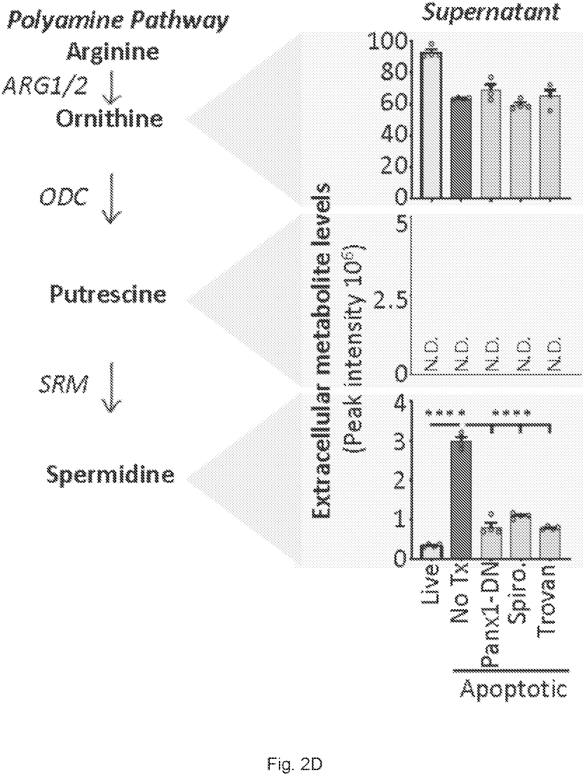

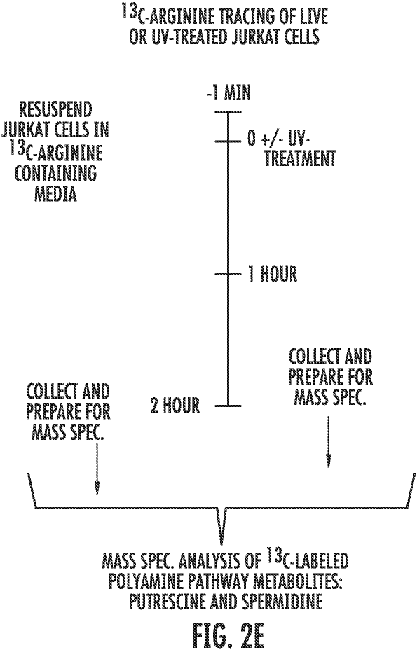

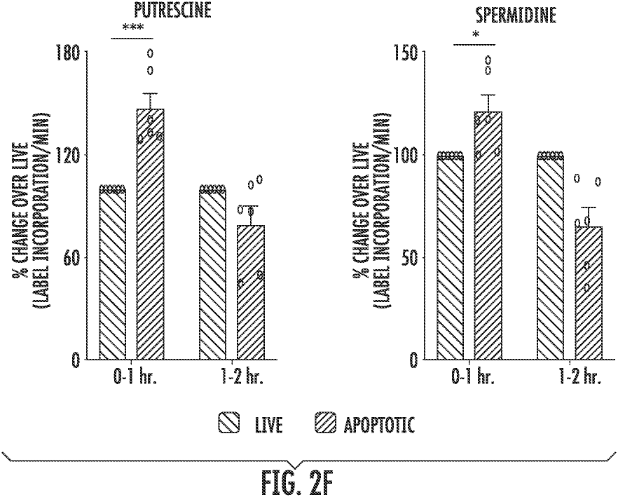

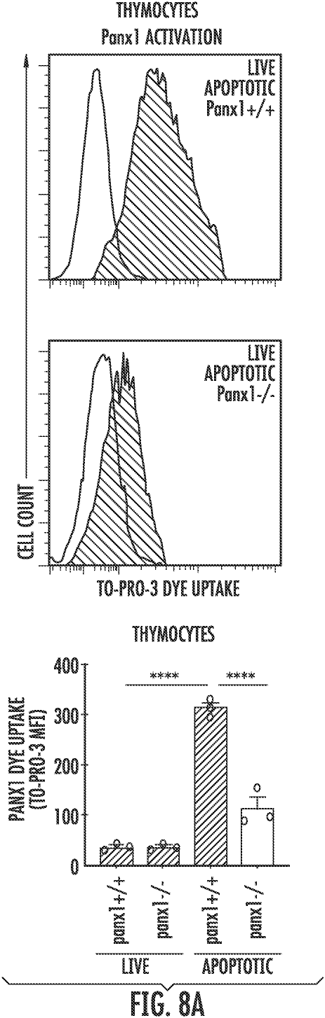

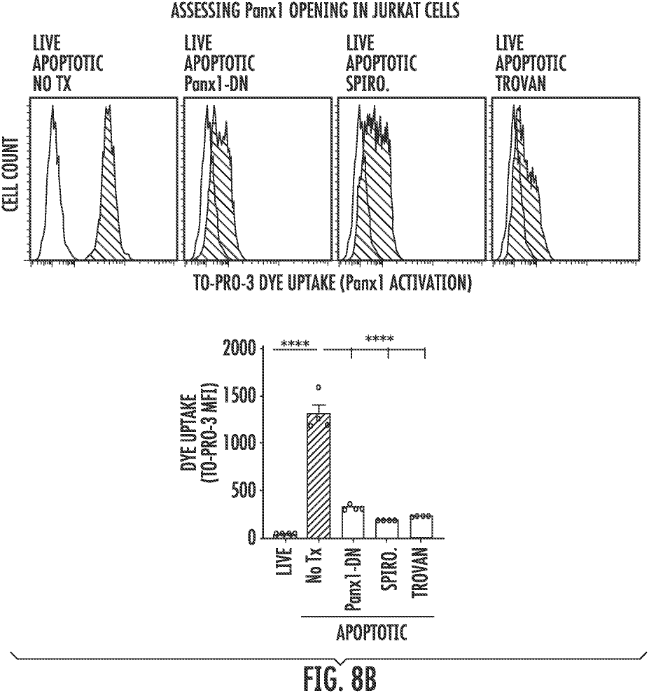

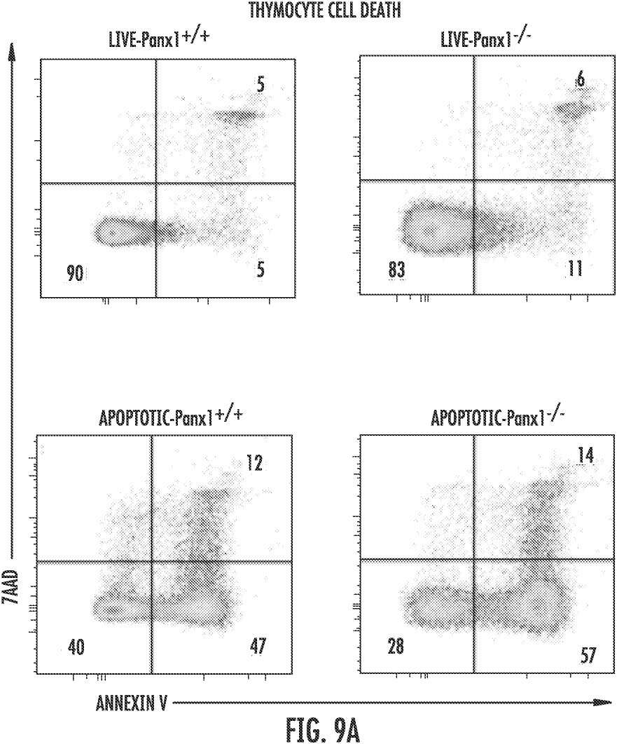

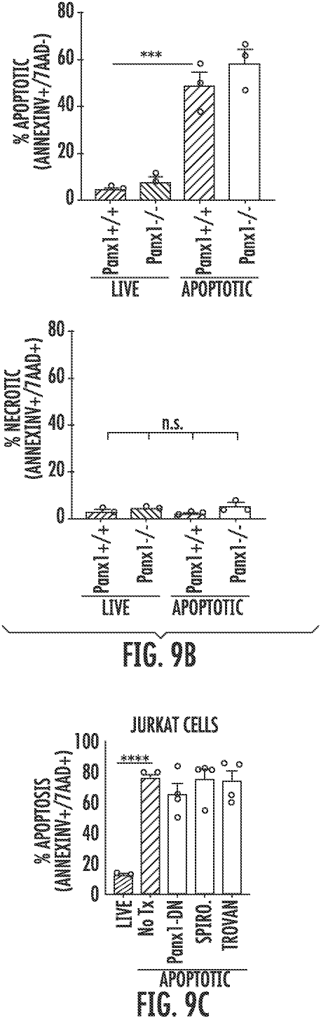

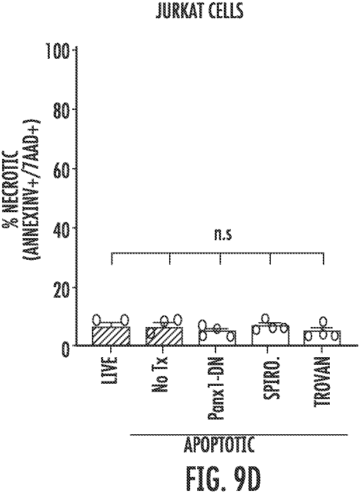

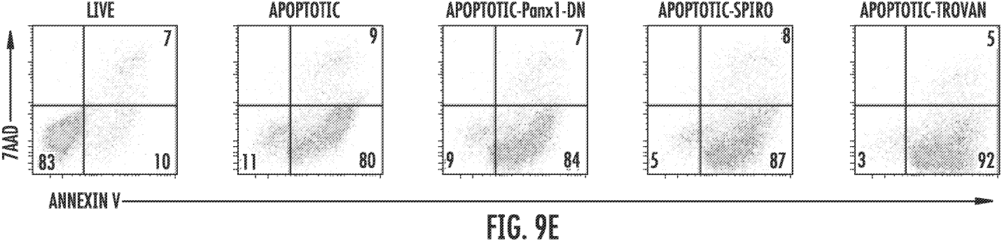

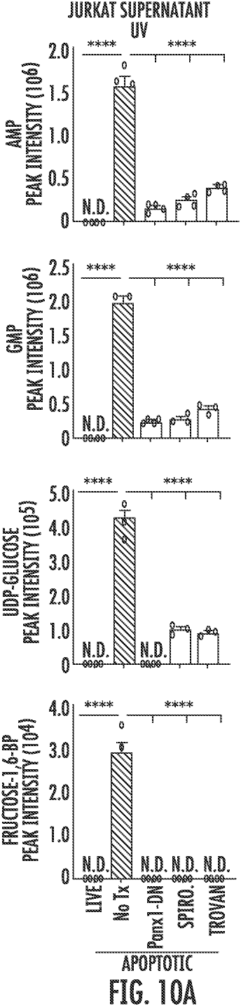

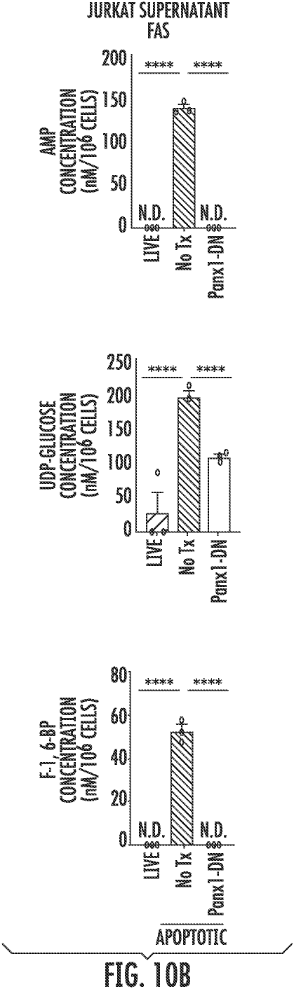

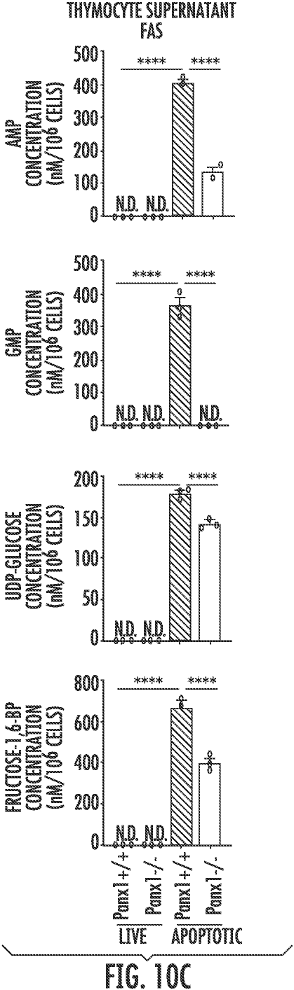

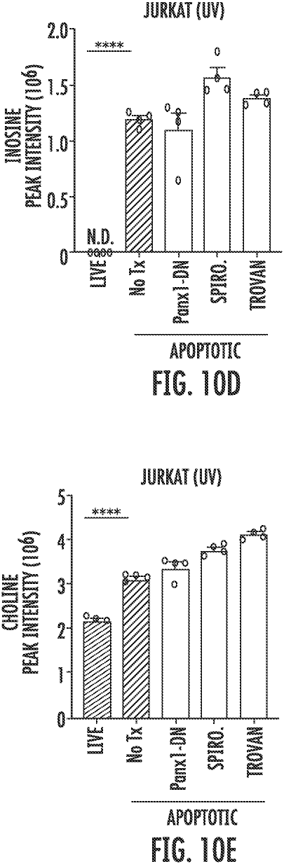

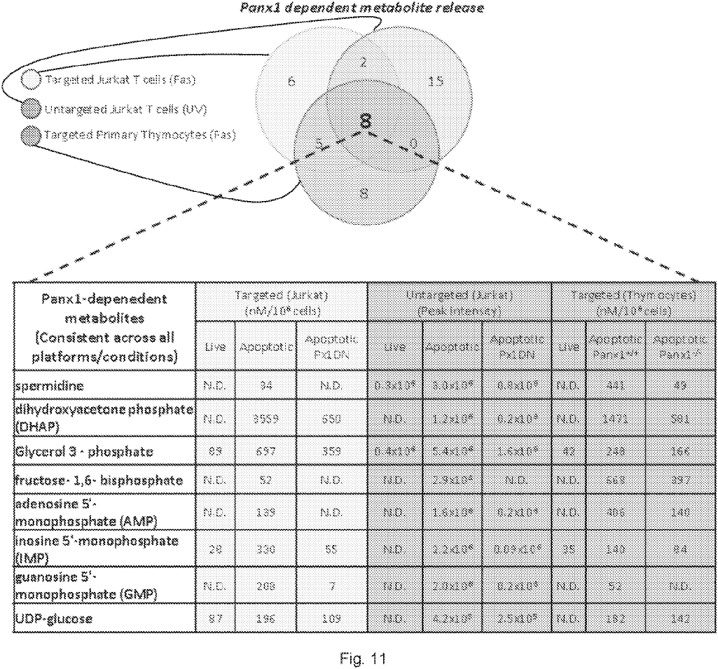

During the above analyses of EXAMPLE 1, it was noted that despite the many cellular metabolites detected in the pellet, only a subset is released; further, even within a known metabolic pathway, only some but not others were released. Such selectivity could arise from either specific channels opening during apoptosis to permeate certain metabolites, and/or continued metabolic activity within the dying cell influencing the secretome. To test specific channels, we focused on pannexin 1 (Panxl) channels that are activated during apoptosis by caspase-mediated cleavage¹⁰ and can conduct ions and small molecules up to 1 kDa in size across the plasma membrane. In a Panxl-dependent manner¹⁰, apoptotic cells (not live cells) take up TO-PRO-3 (671 Daltons) dye, while 7AAD (1.27 kDa) is excluded (FIGS. 8A, 8B). We tested the relevance of Panx1 by genetic and pharmacological approaches. Genetically, we used Jurkat cells expressing a dominant negative Panx1 with a caspase cleavage site mutation¹⁰ (Panxl-DN) or primary thymocytes from Panxl-deficient mice (Panxl^{-/-})¹¹. We also used two pharmacological inhibitors, trovafloxacin (Trovan) and spironolactone (Spiro), which we had previously identified in unbiased screens^{11,12}. Disrupting Panx1 activity per se did not affect apoptosis (FIGS. 9A-9E). Untargeted metabolomics of the supernatants from apoptotic Jurkat cells (UV-induced) with and without Panx1 inhibition revealed that Panx1 contributed to release about 20% of the apoptotic metabolites (25 out of 123) (FIG. 2A, FIG. 10A). The Panxl-dependent metabolites included nucleotides, nucleotide-sugars, and metabolites linked to energy metabolism and amino acid metabolism; interestingly, most have not been previously reported to permeate through Panxl. A similar Panxl-dependent metabolite signature was shared between Jurkat cells and thymocytes; further, as not all apoptotic metabolites released were Panxl-dependent, other mechanisms must also exist (FIGS. 10B-10E). We noted eight shared Panxl-dependent apoptotic metabolites between Jurkat cells and primary thymocytes (FIG. 2B and FIG. 11).

### Example 3

### Testing Whether the Apoptotic Secretome Might Also be Influenced by the Metabolic Activity Within the Dying Cell

To test whether the apoptotic secretome might also be influenced by the metabolic activity within the dying cell, we chose the polyamine pathway for several reasons. First, the polyamine spermidine was released in significant quantities from apoptotic Jurkat cells, macrophages, thymocytes, and epithelial cells after different modes of apoptosis induction (FIG. 2C). Second, among the two metabolites immediately upstream of spermidine, putrescine was not released, while omithine was present comparably in live and apoptotic cell supernatants (FIG. 2D). Third, while exogenous spermidine supplementation can reduce inflammation and improve longevity¹³, spermidine release from apoptotic cells provides the first natural/physiological extracellular source of this polyamine.

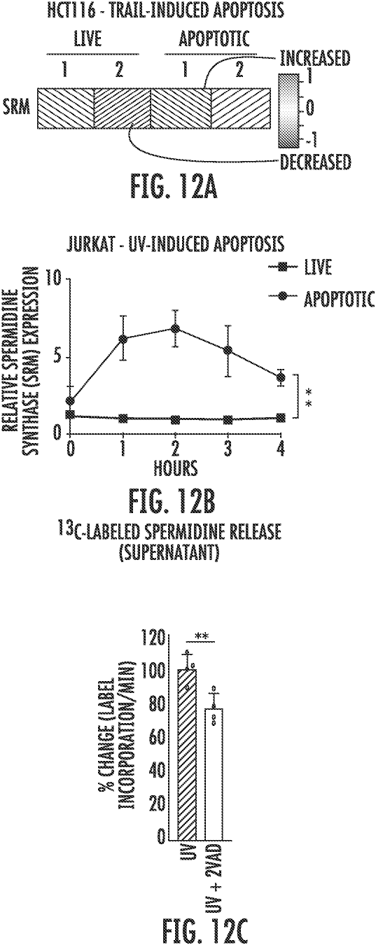

The upstream steps of spermidine generation involve arginine➔ ornithine➔ putrescine➔ spermidine, with each conversion regulated by specific enzymes. A recent report¹⁴ showed that while the majority of mRNA gets degraded in apoptotic HCT-116 cells, a small fraction is ‘retained’. In our re-analysis of this mRNA dataset, the polyamine pathway enzyme transcripts were not degraded during apoptosis, including spermidine synthase (SRM) that converts putrescine to spermidine (FIG. 12A)¹⁴. We confirmed that in apoptotic Jurkat cells, the mRNA for spermidine synthase was retained (FIG. 12B). To address this more directly by metabolic flux labeling, we added ¹³C-Arginine medium to Jurkat cells immediately prior to apoptosis induction, and traced the label incorporation into putrescine and spermidine for the next few hours (FIG. 2E). Apoptotic cells displayed increased ¹³C label incorporation into the polyamine pathway in the first hour, compared to live cells. After normalizing for total label incorporation and focusing on the carbons within the polyamine pathway (see methods), apoptotic cells showed 40% and 25% greater ¹³C label/min incorporation into putrescine and spermidine, respectively, during the first hour (FIG. 2F). Although this dips during the 2^{nd} hour, it was still comparable to live cells. Further, ¹³C-labelled spermidine was detectable in the supernatants of apoptotic cells, and this was partially reduced by inhibition of caspases (FIG. 12C). Interestingly, despite its active generation (revealed by ¹³C labeling), putrescine was not detectable in apoptotic cell supernatants from Jurkat cells (or in the macrophage or thymocytes data set) (FIG. 2D). Thus, apoptotic cells orchestrate the generation and release of select metabolites at least at two levels - caspase-dependent opening of specific channels (Panxl) and continued metabolic activity of certain pathways.

### Example 4

### Testing Whether Released Apoptotic Cell-Derived Metabolites Signal to Alter Gene Expression in Healthy Nearby Cells

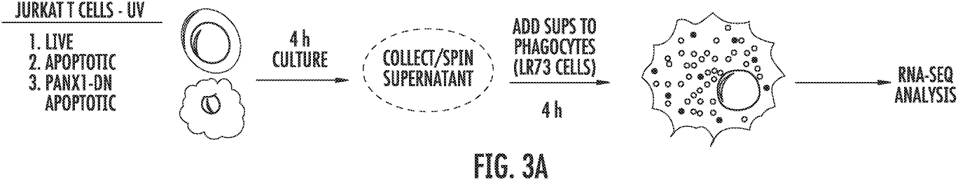

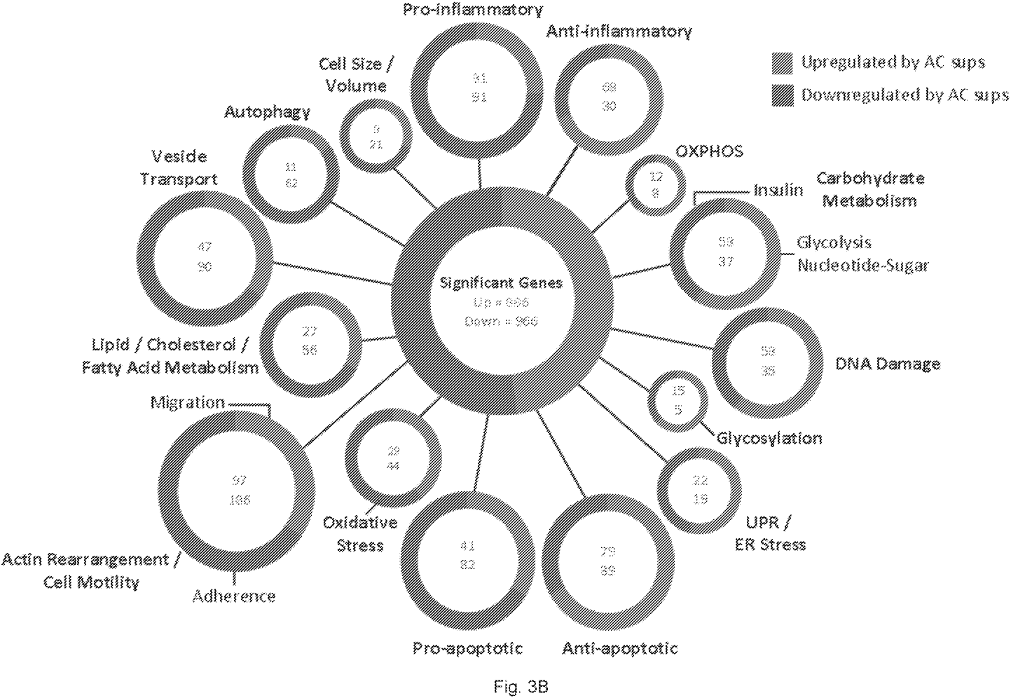

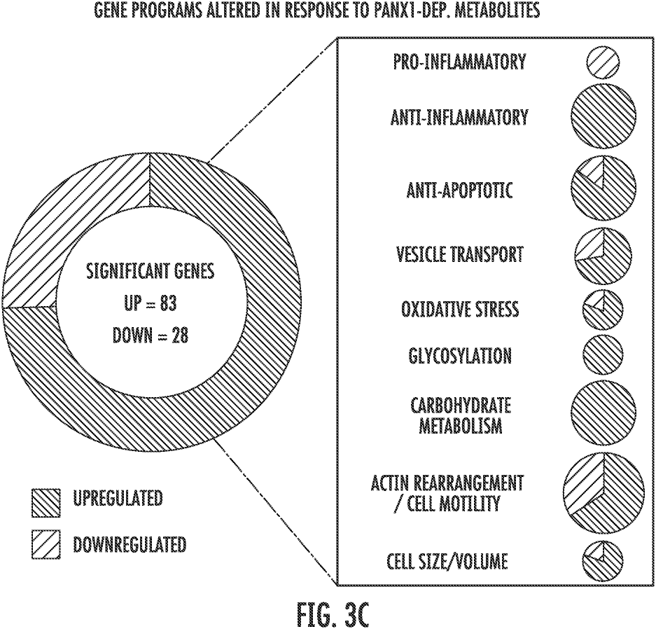

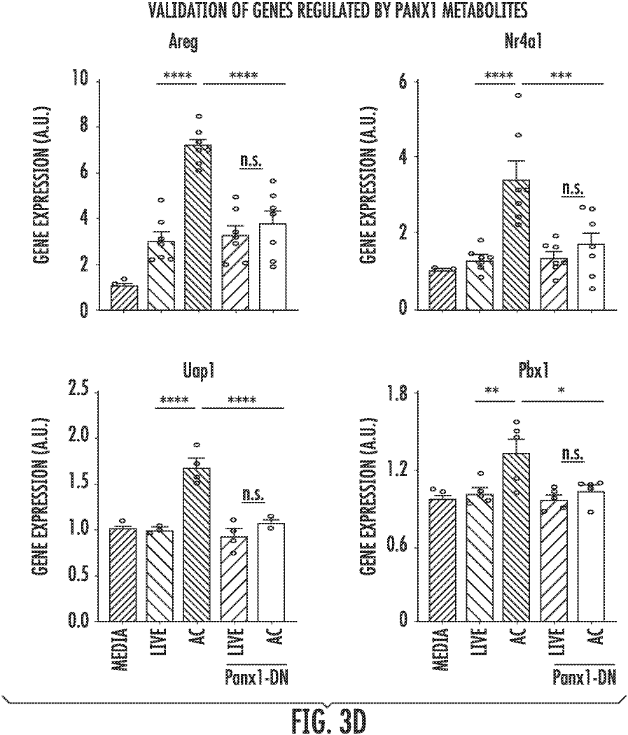

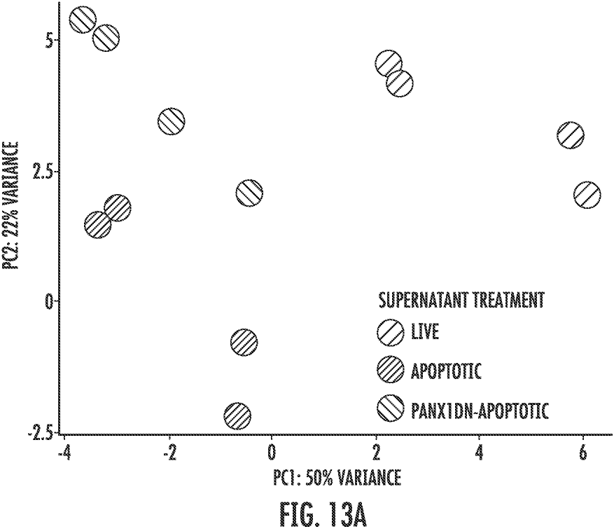

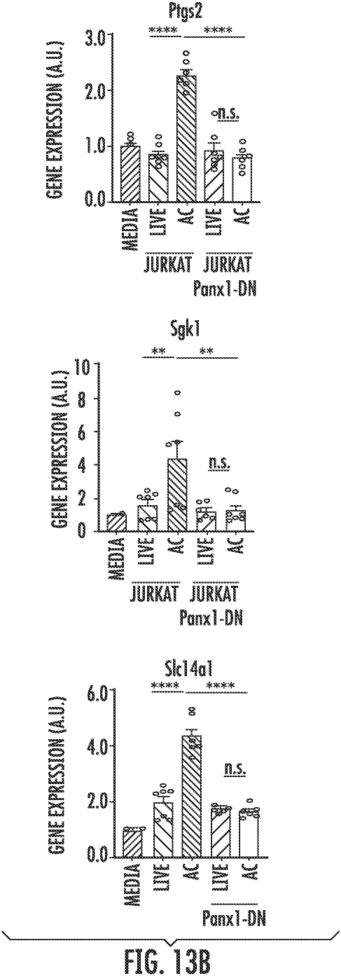

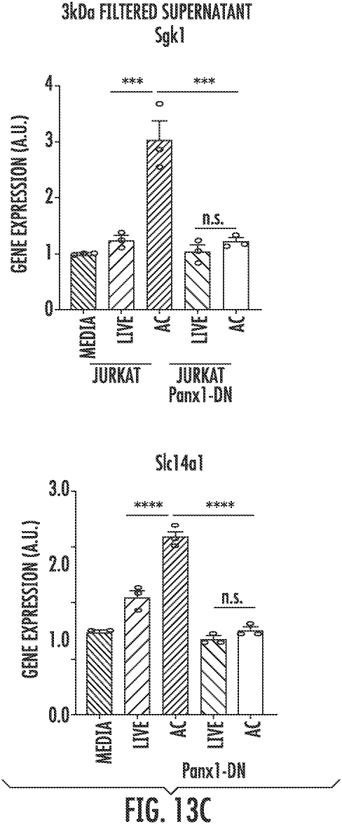

To test whether released apoptotic cell-derived metabolites signal to alter gene expression programs in healthy nearby cells such as phagocytes, we added supernatants from live or apoptotic Jurkat cells (same conditions as untargeted metabolomics) to LR73 cells, a model phagocyte useful in revealing mechanisms/responses after efferocytosis¹⁵⁻⁴⁷ (FIG. 3A). RNAseq analysis of LR73 cells (after 4 hr) indicated distinct transcriptional changes (FIG. 3B and FIG. 13A). Pathway analysis, by hand-curating each of the hits individually, together with commonly used analysis software, revealed that the apoptotic secretome altered gene programs linked to cytoskeletal rearrangements, inflammation, wound healing/tissue repair, anti-apoptotic functions, metabolism, and regulation of cell size within the phagocyte (FIG. 3C), providing a molecular and metabolic basis for how apoptosis may influence essential tissue processes.

By comparing gene programs induced in live cells by supernatants from apoptotic cells versus conditions with genetic inhibition of Panxl, we identified 110 genes as differentially regulated on phagocytes by Panxl-dependent apoptotic metabolites (82 up and 28 down) (FIG. 3C); these include genes involved in anti-inflammatory processes, anti-apoptotic pathways, metabolism, and actin rearrangement (FIG. 3C). Secondary validation via qPCR indicates that Panxl-dependent metabolites can alter genes linked to anti-inflammatory roles in phagocytes (Nr4al, Pbxl)^{18,19}, wound healing (Areg, Ptgs2)^{20,21}, and metabolism (Slc14a1, Sgk1, Uap1)^{15,22} (FIG. 3C and FIG. 13B). Furthermore, filtration of supernatants through 3 kDa filters, prior to addition to phagocytes showed similar gene transcriptional changes (FIG. 13C), ruling out larger proteins or vesicles from dying cells. Thus, metabolites released from apoptotic cells, a subset of which are released in a Panxl-dependent manner, can alter selective gene programs in the surrounding cells that sense these metabolic signals.

### Example 5

### Testing Whether Apoptotic Panx1-Dependent Metabolites can Induce Gene Expression Changes in Tissue Phagocytes In Vivo

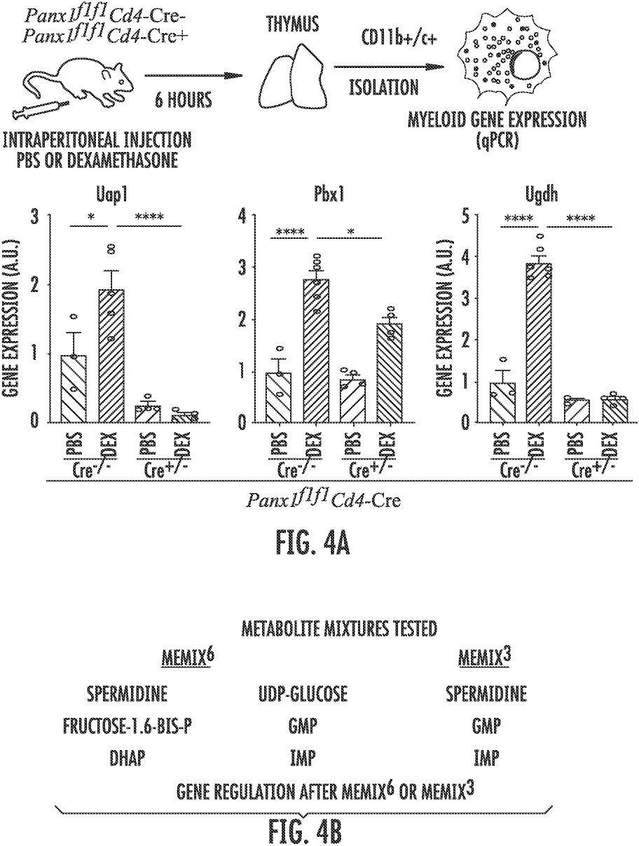

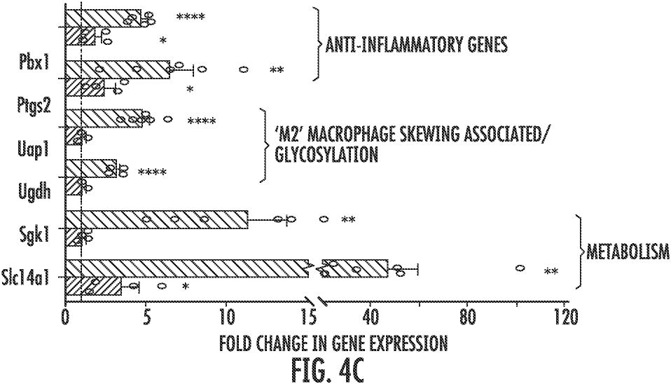

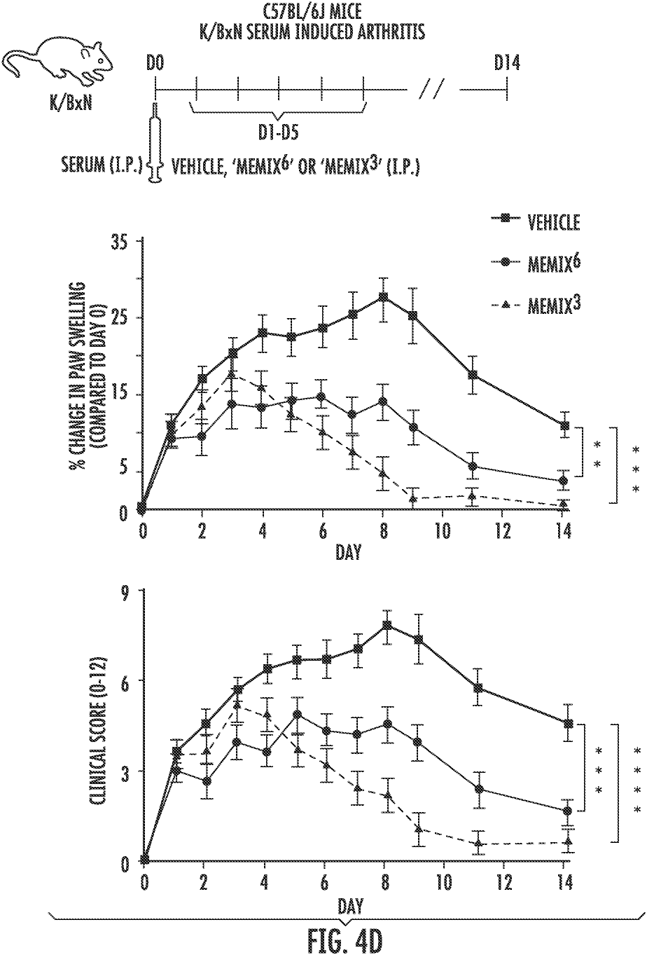

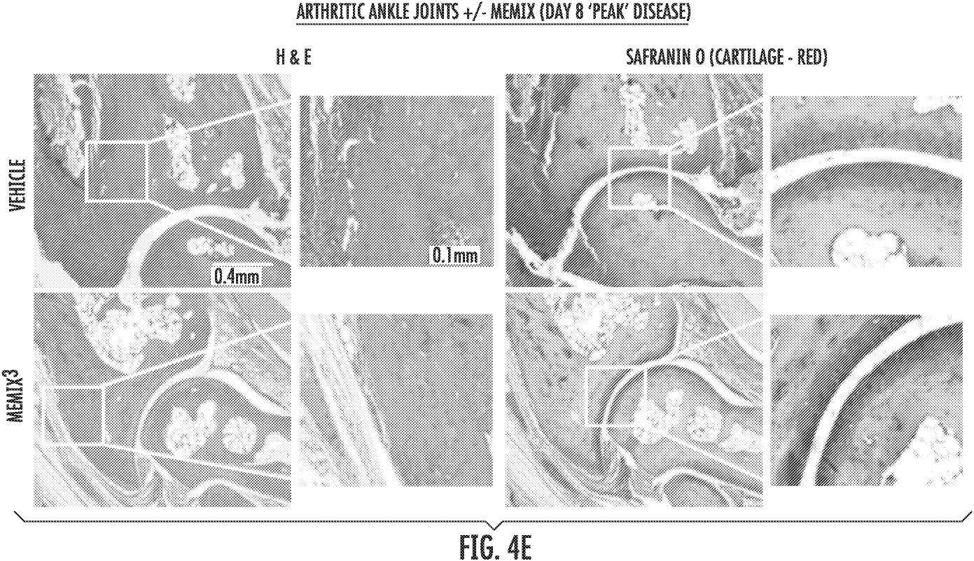

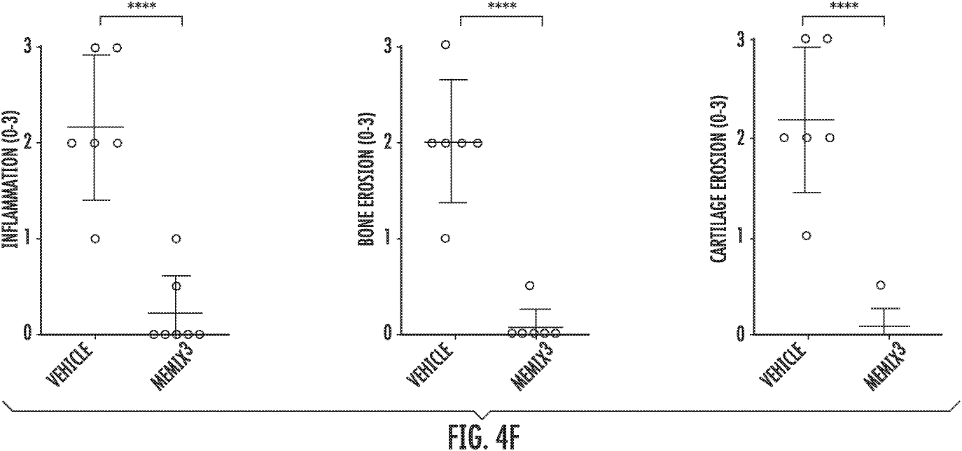

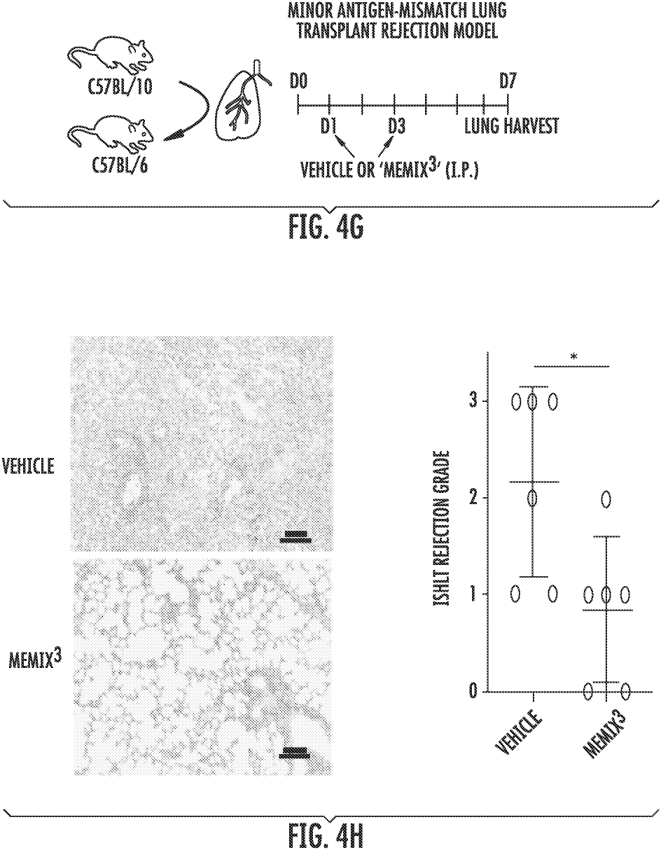

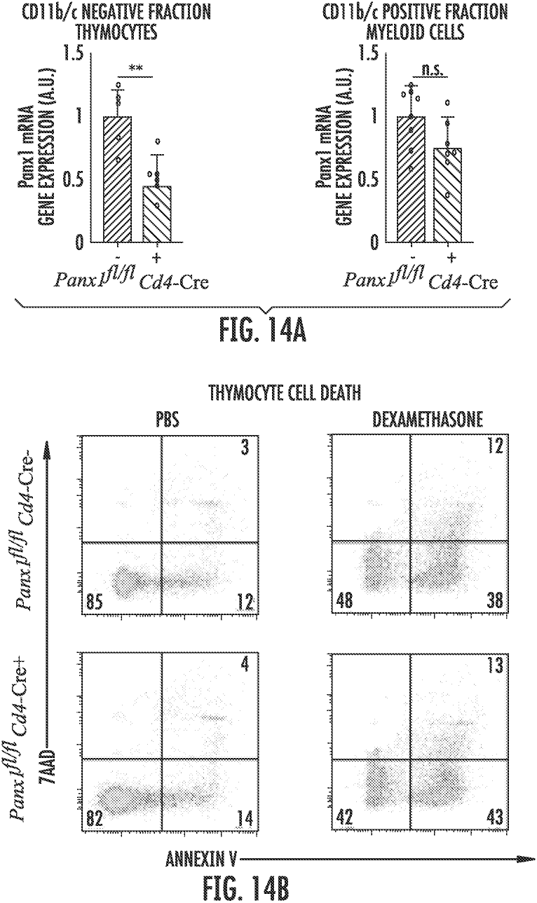

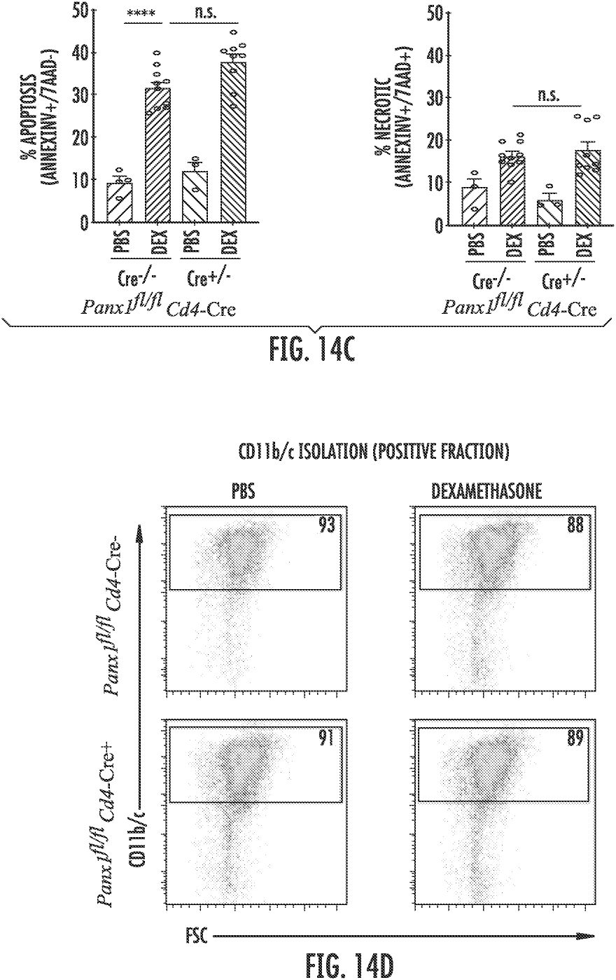

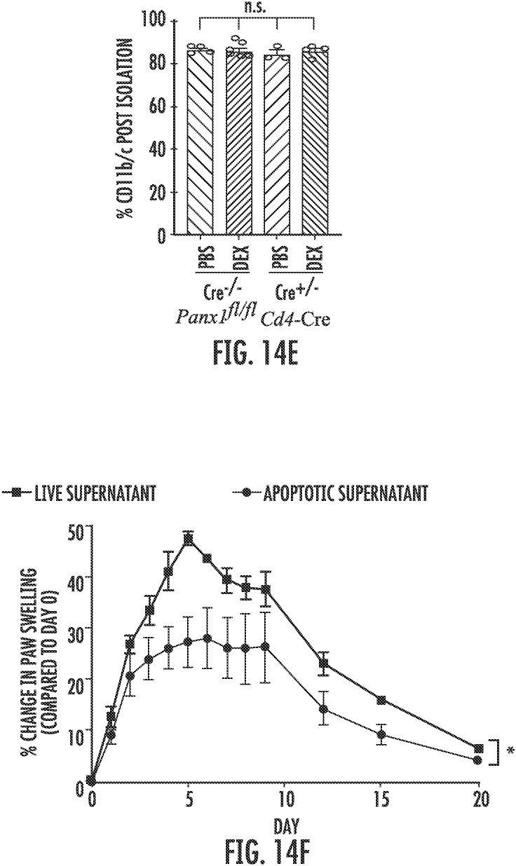

To test whether apoptotic Panxl-dependent metabolites can induce gene expression changes in tissue phagocytes in vivo, we used Panxl^{ƒl/ƒl}Cd4-Cre mice¹¹, where Panx1 is targeted for deletion only within the thymocytes (FIG. 14A left). After confirming that Panx1 was not deleted in the macrophages and dendritic cells (FIG. 14A right), and that comparable dexamethasone-induced thymocyte apoptosis occurs in control and Panxl^{ƒl/ƒl}Cd4-Cre mice (FIGS. 14B and 14C), we isolated CD11b⁺ macrophages and CD11c⁺ dendritic cells from the thymus and analyzed gene expression changes (FIGS. 14D and 14E). In wild-type mice, dexamethasone-induced apoptosis of thymocytes resulted in increased expression of Uap1, Ugdh, and Pbx1 in surrounding live myeloid cells (linked to anti-inflammatory macrophage skewing/glycosylation and IL-10 transcription)^{19,23} (FIG. 4A). This response was attenuated in mice lacking Panx1 channels in the dying thymocytes (FIG. 4A). Thus, apoptotic Panx1-dependent metabolites can induce gene expression changes in the surrounding tissue myeloid cells in vivo.

### Example 6

### Testing Metabolites for Anti-Inflammatory Activity

When tested individually, many of the metabolites failed to strongly induce anti-inflammatory and tissue-repair genes from the RNAseq (not shown). As these metabolites are concurrently released from apoptotic cells (FIGS. 1A-1F), we tested mixtures of 6 of the 8 Panxl-dependent metabolites (FIG. 2B) in two combinations: i) spermidine, fructose-1,6-bisphosphate, dihydroxyacetone phosphate, UDP-glucose, guanosine monophosphate, and inosine monophosphate; and ii) spermidine, guanosine monophosphate, and inosine monophosphate (FIG. 4B). All six have been previously administered in vivo in mice (or rats) without toxicity. We excluded AMP and glycerol-3-phosphate, as AMP can be converted to adenosine, a known anti-inflammatory metabolite, and it was difficult to determine the optimal in vivo dose for glycerol-3-phosphate. The metabolite mixtures were quite potent in inducing gene expression in vitro, including genes linked to anti-inflammatory macrophage skewing/glycosylation (Uap1, Ugdh)²³, IL-10 transcription and inflammation resolution (Pbxl ¹⁹, Ptgs2 ²⁴), and metabolic processes (Slc14a1, Sgkl), some of which have also been shown to be involved in phagocytosis¹⁵ (FIG. 4C). For simplicity, we have denoted the metabolite mixtures as MeMix⁶ and MeMix³ (FIG. 4B).

Given the anti-inflammatory gene signature induced by the metabolites, we next tested MeMix⁶ and/or MeMix³ in attenuating inflammation in vivo in two contexts: a model of inflammatory arthritis, and a model of lung transplant rejection. For arthritis injection, a single injection of the arthritic serum from K/BxN mice into C57BL/6J mice results in inflammation of the joints with progressive arthritic symptoms, followed by disease resolution²⁵. Of relevance to our question, this arthritis model is dependent on myeloid cells²⁵, with apoptosis known to occur during disease. We first asked whether the ‘full’ apoptotic secretome could alleviate inflammation in this arthritis model, and this was the case (FIG. 14F). Administration of MeMix⁶ or MeMix³ after arthritis induction when the disease symptoms are noticeable resulted in significant attenuation of paw swelling and other arthritic parameters, compared to vehicle controls (FIG. 4D). Since fructose 1,6-bisphosphate (FBP) alone can have ameliorative roles in arthritis²⁶, we tested MeMix³, which does not contain FBP. MeMix³ not only alleviated paw swelling and external clinical arthritis parameters, but also significantly protected the joints from inflammation, bone erosion, and cartilage erosion (FIGS. 4E, 4F).

We also tested Memix³ in a lung transplant rejection model, where local innate and adaptive immune responses orchestrated by graft-resident antigen presenting myeloid cells, dictate graft acceptance or rejection. We transplanted C57BL/10 left lung allografts to a minor antigen mismatched C57BL/6 recipient (FIG. 4G)²⁷, treating the graft recipients with Memix³ or saline vehicle control on post-operative days 1 and 3. On day 7 post-engraftment, saline-treated control mice showed severe acute rejection of allografts²⁸. Remarkably, Memix³ treated mice had only minimal inflammation in the transplanted lungs (FIG. 4H), suggestive of amelioration of lung rejection. Complementary flow cytometric analysis of the lung showed reduced CD4 and CD8 cells in the transplanted lung of mice treated with Memix ³. Thus, a subset of apoptotic metabolites can be harnessed for beneficial effects in two different inflammatory settings in vivo.

### Discussion of Examples

Collectively, the data presented here advance several concepts. Specific metabolites that are released from apoptotic cells (different cell types and modes of apoptosis induction) were identified. While it is not desired to be bound by any particular theory of operation, the specificity could arise from metabolic changes in the apoptotic cells (e.g. sustained spermidine production), and/or the opening of specific channels (e.g. Panx1). Also, it was observed that apoptotic cells are not inert awaiting removal. Rather, apoptotic cells, via metabolites as “good-bye” signals, modulate multiple gene programs in the neighboring cells within a tissue. The ability of a cocktail of apoptotic metabolites to attenuate arthritic symptoms and lung transplantation rejection was also observed, indicating that it is possible to harness the beneficial therapeutic properties of apoptosis in particular inflammatory conditions.

TABLE 1

Jurkat UV metabolite release

| Super Pathway | Sub Pathway | Biochemical | Platform | Fold Change (FAp/FL) | pval | qvalue |
|---|---|---|---|---|---|---|
| Lipid | Lysolipid | 1-oleoyl-GPC (18:1) | LC/MS pos late | 2.74 | 0.0003 | 0.0002 |
| Lipid | Fatty Acid, Dicarboxylate | 2-hydroxyglutarate | LC/MS polar | 3.06 | 0.0075 | 0.0026 |
| Nucleotide | Purine Metabolism, Adenine containing | 2′-deoxyadenosine 5′-monophosphate | LC/MS neg | 20.95 | 0.0000 | 0.0000 |
| Nucleotide | Pyrimidine Metabolism, Cytidine containing | 2′-deoxycytidine | LC/MS pos early | 4.21 | 0.0000 | 0.0000 |
| Nucleotide | Pyrimidine Metabolism, Cytidine containing | 2′-deoxycytidine 5′-monophosphate | LC/MS pos early | 1.54 | 0.0015 | 0.0007 |
| Nucleotide | Purine Metabolism, Guanine containing | 2′-deoxyguanosine | LC/MS neg | 3.12 | 0.0004 | 0.0002 |
| Nucleotide | Purine Metabolism, (Hypo)Xanthine/Inosine containing | 2′-deoxyinosine | LC/MS pos early | 2.38 | 0.0001 | 0.0001 |
| Lipid | Mevalonate Metabolism | 3 -hydroxy-3 -methylglutarate | LC/MS polar | 1.70 | 0.0004 | 0.0002 |
| Nucleotide | Pyrimidine Metabolism, Cytidine containing | 3-methylcytidine | LC/MS pos early | 1.53 | 0.0013 | 0.0006 |
| Amino Acid | Alanine and Aspartate Metabolism | 3 -sulfo-L-alanine | LC/MS polar | 2.30 | 0.0094 | 0.0031 |
| Amino Acid | Guanidino and Acetamido Metabolism | 4-guanidinobutanoate | LC/MS pos early | 1.42 | 0.0012 | 0.0006 |
| Amino Acid | Phenylalanine and Tyrosine Metabolism | 4-hydroxyphenylpyruvate | LC/MS neg | 4.22 | 0.0040 | 0.0015 |
| Nucleotide | Pyrimidine Metabolism, Cytidine containing | 5-methyl-2′-deoxycytidine | LC/MS pos early | 1.65 | 0.0151 | 0.0047 |
| Lipid | Fatty Acid Metabolism(Acyl Carnitine) | acetylcarnitine | LC/MS pos early | 2.66 | 0.0371 | 0.0101 |
| Lipid | Neurotransmitter | acetylcholine | LC/MS pos early | 19.50 | 0.0004 | 0.0002 |
| Nucleotide | Purine Metabolism, Adenine containing | adenine | LC/MS pos early | 4.63 | 0.0094 | 0.0031 |
| Nucleotide | Purine Metabolism, Adenine containing | adenosine 5′-monophosphate (AMP) | LC/MS pos early | 14.95 | 0.0001 | 0.0001 |
| Energy | TCA Cycle | alpha-ketoglutarate | LC/MS polar | 2.69 | 0.0001 | 0.0001 |
| Carbohydrate | Pentose Metabolism | arabonate/xylonate | LC/MS polar | 1.48 | 0.0146 | 0.0046 |
| Amino Acid | Urea cycle; Arginine and Proline Metabolism | arginine | LC/MS pos early | 1.04 | 0.0293 | 0.0085 |
| Amino Acid | Urea cycle; Arginine and Proline Metabolism | argininosuccinate | LC/MS pos early | 6.25 | 0.0000 | 0.0000 |
| Lipid | Fatty Acid Metabolism (also BCAA Metabolism) | butyrylcarnitine | LC/MS pos early | 3.83 | 0.0047 | 0.0017 |
| Lipid | Carnitine Metabolism | carnitine | LC/MS pos early | 2.19 | 0.0003 | 0.0002 |
| Lipid | Phospholipid Metabolism | choline | LC/MS pos early | 1.45 | 0.0001 | 0.0001 |
| Lipid | Phospholipid Metabolism | choline phosphate | LC/MS pos early | 12.15 | 0.0000 | 0.0000 |
| Amino Acid | Creatine Metabolism | creatine | LC/MS pos early | 5.26 | 0.0000 | 0.0000 |
| Amino Acid | Methionine, Cysteine, SAM and Taurine Metabolism | cystathionine | LC/MS pos early | 2.52 | 0.0326 | 0.0092 |
| Amino Acid | Methionine, Cysteine, SAM and Taurine Metabolism | cysteine | LC/MS pos early | 1.78 | 0.0000 | 0.0000 |
| Amino Acid | Methionine, Cysteine, SAM and Taurine Metabolism | cysteine s-sulfate | LC/MS polar | 2.12 | 0.0000 | 0.0000 |
| Amino Acid | Methionine, Cysteine, SAM and Taurine Metabolism | cysteine sulfinic acid | LC/MS pos early | 2.02 | 0.0046 | 0.0017 |
| Amino Acid | Glutathione Metabolism | cysteine-glutathione disulfide | LC/MS pos early | 4.22 | 0.0000 | 0.0000 |
| Nucleotide | Pyrimidine Metabolism, Cytidine containing | cytidine | LC/MS neg | 99.36 | 0.0000 | 0.0000 |
| Lipid | Phospholipid Metabolism | cytidine 5′-diphosphocholine | LC/MS pos early | 11.87 | 0.0000 | 0.0000 |
| Nucleotide | Pyrimidine Metabolism, Cytidine containing | cytidine 5′-monophosphate (5′-CMP) | LC/MS pos early | 16.36 | 0.0000 | 0.0000 |
| Lipid | Carnitine Metabolism | deoxycarnitine | LC/MS pos early | 3.25 | 0.0004 | 0.0002 |
| Carbohydrate | Glycolysis, Gluconeogenesis, and Pyruvate Metabolism | dihydroxyacetone phosphate (DHAP) | LC/MS neg | 9.87 | 0.0002 | 0.0001 |
| Amino Acid | Urea cycle; Arginine and Proline Metabolism | dimethyl arginine (SDMA + ADMA) | LC/MS pos early | 1.83 | 0.0000 | 0.0000 |
| Amino Acid | Glycine, Serine and Threonine Metabolism | dimethylglycine | LC/MS pos early | 2.29 | 0.0135 | 0.0043 |
| Lipid | Polyunsaturated Fatty Acid (n3 and n6) | docosahexaenoate (DHA; 22:6n3) | LC/MS neg | 1.97 | 0.0013 | 0.0006 |
| Lipid | Long Chain Fatty Acid | eicosenoate (20:1) | LC/MS neg | 2.49 | 50.0001 | 0.0001 |
| Carbohydrate | Aminosugar Metabolism | erythronate* | LC/MS polar | 1.13 | 0.0349 | 0.0096 |
| Carbohydrate | Fructose, Mannose and Galactose Metabolism | fructose | LC/MS polar | 1.25 | 0.0338 | 0.0093 |
| Carbohydrate | Glycolysis, Gluconeogenesis, and Pyruvate Metabolism | fructose 1,6-diphosphate | LC/MS neg | | | |
| Energy | TCA Cycle | fumarate | LC/MS polar | 5.39 | 0.0000 | 0.0000 |
| Peptide | Gamma-glutamyl Amino Acid | gamma-glutamylglutamine | LC/MS pos early | 1.51 | 0.0036 | 0.0014 |
| Peptide | Gamma-glutamyl Amino Acid | gamma-glutamylmethionine | LC/MS pos early | 2.02 | 0.0237 | 0.0071 |
| Peptide | Gamma-glutamyl Amino Acid | gamma-glutamylthreonine* | LC/MS pos early | 4.15 | 0.0003 | 0.0002 |
| Carbohydrate | Glycolysis, Gluconeogenesis, and Pyruvate Metabolism | glucose | LC/MS polar | 1.17 | 0.0025 | 0.0010 |
| Amino Acid | Glutamate Metabolism | glutamate | LC/MS pos early | 1.52 | 0.0000 | 0.0000 |
| Amino Acid | Glutamate Metabolism | glutamine | LC/MS pos early | 1.15 | 0.0361 | 0.0099 |
| Amino Acid | Glutathione Metabolism | glutathione, oxidized (GSSG) | LC/MS pos early | 3.20 | 0.0002 | 0.0001 |
| Lipid | Glycerolipid Metabolism | glycerol 3-phosphate | LC/MS pos early | 14.17 | 0.0000 | 0.0000 |
| Lipid | Phospholipid Metabolism | glycerophosphoethanolamine | LC/MS pos early | 37.37 | 0.0001 | 0.0001 |
| Lipid | Glycerolipid Metabolism | glycerophosphoglycerol | LC/MS polar | 5.32 | 0.0006 | 0.0003 |
| Lipid | Phospholipid Metabolism | glycerophosphoinositol* | LC/MS polar | 16.30 | 0.0000 | 0.0000 |
| Lipid | Phospholipid Metabolism | glycerophosphorylcholine (GPC) | LC/MS pos early | 17.82 | 0.0000 | 0.0000 |
| Peptide | Dipeptide | glycylvaline | LC/MS pos early | 9.36 | 0.0001 | 0.0001 |
| Nucleotide | Purine Metabolism, Guanine containing | guanine | LC/MS pos early | 203.22 | 0.0003 | 0.0002 |
| Nucleotide | Purine Metabolism, Guanine containing | guanosine | LC/MS neg | 11.79 | 0.0000 | 0.0000 |
| Nucleotide | Purine Metabolism, Guanine containing | guanosine 5′- monophosphate (5′-GMP) | LC/MS pos early | 8.66 | 0.0000 | 0.0000 |
| Cofactors and Vitamins | Ascorbate and Aldarate Metabolism | gulonic acid* | LC/MS polar | 2.71 | 0.0496 | 0.0132 |
| Amino Acid | Methionine, Cysteine, SAM and Taurine Metabolism | hypotaurine | LC/MS pos early | 35.43 | 0.0000 | 0.0000 |
| Nucleotide | Purine Metabolism, (Hypo)Xanthine/Inosine containing | hypoxanthine | LC/MS neg | 3.60 | 0.0000 | 0.0000 |
| Nucleotide | Purine Metabolism, (Hypo)Xanthine/Inosine containing | inosine | LC/MS neg | 5.14 | 0.0000 | 0.0000 |
| Nucleotide | Purine Metabolism, (Hypo)Xanthine/Inosine containing | inosine 5′-monophosphate (IMP) | LC/MS pos early | 28.84 | 0.0000 | 0.0000 |
| Amino Acid | Leucine, Isoleucine and Valine Metabolism | isoleucine | LC/MS pos early | 1.12 | 0.0017 | 0.0007 |
| Amino Acid | Leucine, Isoleucine and Valine Metabolism | leucine | LC/MS pos early | 1.24 | 0.0000 | 0.0000 |
| Lipid | Polyunsaturated Fatty Acid (n3 and n6) | linoleate (18:2n6) | LC/MS neg | 1.64 | 0.0017 | 0.0007 |
| Lipid | Polyunsaturated Fatty Acid (n3 and n6) | linolenate [alpha or gamma; (18:3n3 or 6)] | LC/MS neg | 1.91 | 0.0019 | 0.0008 |
| Amino Acid | Lysine Metabolism | lysine | LC/MS pos early | 1.22 | 0.0017 | 0.0007 |
| Energy | TCA Cycle | malate | LC/MS polar | 8.33 | 0.0000 | 0.0000 |
| Carbohydrate | Fructose, Mannose and Galactose Metabolism | mannitol/sorbitol | LC/MS polar | 2.25 | 0.0015 | 0.0007 |
| Amino Acid | Methionine, Cysteine, SAM and Taurine Metabolism | methionine | LC/MS pos early | 1.20 | 0.0115 | 0.0037 |
| Amino Acid | Methionine, Cysteine, SAM and Taurine Metabolism | methionine sulfoxide | LC/MS pos early | 1.16 | 0.0016 | 0.0007 |
| Lipid | Fatty Acid Metabolism (also BCAA Metabolism) | methylmalonate (MMA) | LC/MS polar | 1.68 | 0.0019 | 0.0008 |
| Amino Acid | Urea cycle; Arginine and Proline Metabolism | N-acetylarginine | LC/MS pos early | 2.02 | 0.0000 | 0.0000 |
| Amino Acid | Alanine and Aspartate Metabolism | N-acetylasparagine | LC/MS pos early | 2.70 | 0.0001 | 0.0001 |
| Amino Acid | Alanine and Aspartate Metabolism | N-acetylaspartate (NAA) | LC/MS polar | 7.35 | 0.0000 | 0.0000 |
| Carbohydrate | Aminosugar Metabolism | N-acetylglucosamine/N-acetylgalactosamine | LC/MS pos early | 6.04 | 0.0001 | 0.0001 |
| Amino Acid | Glutamate Metabolism | N-acetylglutamate | LC/MS polar | 3.30 | 0.0003 | 0.0002 |
| Amino Acid | Glutamate Metabolism | N-acetylglutamine | LC/MS pos early | 3.45 | 0.0000 | 0.0000 |
| Amino Acid | Histidine Metabolism | N-acetylhistidine | LC/MS pos early | 2.58 | 0.0003 | 0.0002 |
| Amino Acid | Leucine, Isoleucine and Valine Metabolism | N-acetylisoleucine | LC/MS neg | 2.76 | 0.0002 | 0.0001 |
| Amino Acid | Leucine, Isoleucine and Valine Metabolism | N-acetylleucine | LC/MS neg | 2.48 | 0.0000 | 0.0000 |
| Amino Acid | Methionine, Cysteine, SAM and Taurine Metabolism | N-acetylmethionine | LC/MS neg | 1.70 | 0.0013 | 0.0006 |
| Carbohydrate | Aminosugar Metabolism | N-acetylneuraminate | LC/MS pos early | 4.18 | 0.0002 | 0.0001 |
| Amino Acid | Phenylalanine and Tyrosine Metabolism | N-acetylphenylalanine | LC/MS neg | 2.76 | 0.0002 | 0.0001 |
| Amino Acid | Urea cycle; Arginine and Proline Metabolism | N-acetylproline | LC/MS pos early | 2.15 | 0.0024 | 0.0010 |
| Amino Acid | Glycine, Serine and Threonine Metabolism | N-acetylserine | LC/MS polar | 3.59 | 0.0000 | 0.0000 |
| Amino Acid | Glycine, Serine and Threonine Metabolism | N-acetylthreonine | LC/MS neg | 4.17 | 0.0000 | 0.0000 |
| Nucleotide | Pyrimidine Metabolism, Orotate containing | N-carbamoylaspartate | LC/MS polar | 1.31 | 0.0303 | 0.0087 |
| Amino Acid | Phenylalanine and Tyrosine Metabolism | N-formylphenylalanine | LC/MS neg | 1.87 | 0.0067 | 0.0024 |
| Nucleotide | Purine Metabolism, (Hypo)Xanthine/Inosine containing | N1-methylinosine | LC/MS pos early | 1.47 | 0.0085 | 0.0029 |
| Amino Acid | Lysine Metabolism | N2-acetyllysine/N6-acetyllysine | LC/MS pos early | 2.62 | 0.0026 | 0.0010 |
| Nucleotide | Purine Metabolism, Guanine containing | N2,N2-dimethylguanosine | LC/MS pos early | 1.63 | 0.0048 | 0.0017 |
| Cofactors and Vitamins | Nicotinate and Nicotinamide Metabolism | nicotinamide | LC/MS pos early | 1.25 | 0.0061 | 0.0022 |
| Cofactors and Vitamins | Nicotinate and Nicotinamide Metabolism | nicotinamide riboside | LC/MS pos early | 2.66 | 0.0040 | 0.0015 |
| Nucleotide | Pyrimidine Metabolism, Orotate containing | orotidine | LC/MS polar | 5.30 | 0.0007 | 0.0003 |
| Lipid | Long Chain Fatty Acid | palmitoleate (16:1n7) | LC/MS neg | 2.45 | 0.0002 | 0.0001 |
| Amino Acid | Phenylalanine and Tyrosine Metabolism | phenylacetylglycine | LC/MS neg | 1.86 | 0.0000 | 0.0000 |
| Amino Acid | Phenylalanine and Tyrosine Metabolism | phenylalanine | LC/MS pos early | 1.14 | 0.0048 | 0.0017 |
| Amino Acid | Lysine Metabolism | pipecolate | LC/MS pos early | 1.28 | 0.0490 | 0.0131 |
| Amino Acid | Urea cycle; Arginine and Proline Metabolism | proline | LC/MS pos early | 1.16 | 0.0088 | 0.0029 |
| Peptide | Dipeptide | prolylglycine | LC/MS pos early | 7.37 | 0.0000 | 0.0000 |
| Lipid | Fatty Acid Metabolism (also BCAA Metabolism) | propionylcarnitine | LC/MS pos early | 2.50 | 0.0011 | 0.0005 |
| Carbohydrate | Pentose Metabolism | ribonate | LC/MS polar | 1.31 | 0.0336 | 0.0093 |
| Amino Acid | Glycine, Serine and Threonine Metabolism | serine | LC/MS pos early | 1.26 | 0.0005 | 0.0003 |
| Amino Acid | Polyamine Metabolism | spermidine | LC/MS pos early | 8.91 | 0.0000 | 0.0000 |
| Energy | TCA Cycle | succinate | LC/MS polar | 1.55 | 0.0082 | 0.0028 |
| Amino Acid | Methionine, Cysteine, SAM and Taurine Metabolism | taurine | LC/MS polar | 19.01 | 0.0000 | 0.0000 |
| Amino Acid | Tryptophan Metabolism | thioproline | LC/MS pos early | 1.40 | 0.0018 | 0.0008 |
| Amino Acid | Glycine, Serine and Threonine Metabolism | threonine | LC/MS pos early | 1.20 | 0.0047 | 0.0017 |
| Nucleotide | Pyrimidine Metabolism, Thymine containing | thymidine | LC/MS neg | 24.22 | 0.0000 | 0.0000 |
| Amino Acid | Tryptophan Metabolism | tryptamine | LC/MS pos early | 1.46 | 0.0119 | 0.0038 |
| Amino Acid | Tryptophan Metabolism | tryptophan | LC/MS pos early | 1.26 | 0.0002 | 0.0001 |
| Amino Acid | Phenylalanine and Tyrosine Metabolism | tyrosine | LC/MS pos early | 1.14 | 0.0005 | 0.0003 |
| Carbohydrate | Nucleotide Sugar | UDP-galactose | LC/MS polar | 12.83 | 0.0000 | 0.0000 |
| Carbohydrate | Nucleotide Sugar | UDP-glucose | LC/MS polar | 5.03 | 0.0001 | 0.0001 |
| Carbohydrate | Nucleotide Sugar | UDP-N-acetylgalactosamine | LC/MS polar | 16.96 | 0.0000 | 0.0000 |
| Carbohydrate | Nucleotide Sugar | UDP-N-acetylglucosamine | LC/MS polar | 15.14 | 0.0000 | 0.0000 |
| Nucleotide | Pyrimidine Metabolism, Uracil containing | uridine | LC/MS neg | 25.03 | 0.0000 | 0.0000 |
| Nucleotide | Pyrimidine Metabolism, Uracil containing | uridine 5′-monophosphate (UMP) | LC/MS polar | 9.39 | 0.0000 | 0.0000 |
| Amino Acid | Leucine, Isoleucine and Valine Metabolism | valine | LC/MS pos early | 1.26 | 0.0006 | 0.0003 |

TABLE 2

Metabolite supernatant enrichment and pellet decrease

| Super Pathway | Sub Pathway | Biochemical | Fold Change (FAp/FL)-Supe | Fold Change (FAp/FL)-Cell |
|---|---|---|---|---|
| Amino Acid | Alanine and Aspartate Metabolism | N-acetylaspartate (NAA) | 7.35 | 0.10 |
| Amino Acid | Alanine and Aspartate Metabolism | N-acetylasparagine | 2.70 | 0.64 |
| Amino Acid | Creatine Metabolism | creatine | 5.26 | 0.21 |
| Amino Acid | Glutamate Metabolism | N-acetylglutamine | 3.45 | 0.43 |
| Amino Acid | Glutamate Metabolism | N-acetylglutamate | 3.30 | 0.12 |
| Amino Acid | Glutamate Metabolism | glutamate | 1.52 | 0.23 |
| Amino Acid | Glutamate Metabolism | glutamine | 1.15 | 0.47 |
| Amino Acid | Glutathione Metabolism | glutathione, oxidized (GSSG) | 3.20 | 0.17 |
| Amino Acid | Glycine, Serine and Threonine Metabolism | N-acetylthreonine | 4.17 | 0.16 |
| Amino Acid | Glycine, Serine and Threonine Metabolism | N-acetylserine | 3.59 | 0.11 |
| Amino Acid | Glycine, Serine and Threonine Metabolism | dimethylglycine | 2.29 | 0.46 |
| Amino Acid | Glycine, Serine and Threonine Metabolism | serine | 1.26 | 0.68 |
| Amino Acid | Glycine, Serine and Threonine Metabolism | threonine | 1.20 | 0.69 |
| Amino Acid | Guanidino and Acetamido Metabolism | 4-guanidinobutanoate | 1.42 | 0.28 |
| Amino Acid | Histidine Metabolism | N-acetylhistidine | 2.58 | 0.49 |
| Amino Acid | Leucine, Isoleucine and Valine Metabolism | valine | 1.26 | 0.76 |
| Amino Acid | Leucine, Isoleucine and Valine Metabolism | leucine | 1.24 | 0.70 |
| Amino Acid | Leucine, Isoleucine and Valine Metabolism | isoleucine | 1.12 | 0.71 |
| Amino Acid | Lysine Metabolism | pipecolate | 1.28 | 0.08 |
| Amino Acid | Methionine, Cysteine, SAM and Taurine Metabolism | hypotaurine | 35.43 | 0.06 |
| Amino Acid | Methionine, Cysteine, SAM and Taurine Metabolism | taurine | 19.01 | 0.08 |
| Amino Acid | Methionine, Cysteine, SAM and Taurine Metabolism | cystathionine | 2.52 | 0.08 |
| Amino Acid | Methionine, Cysteine, SAM and Taurine Metabolism | cysteine sulfinic acid | 2.02 | 0.48 |
| Amino Acid | Methionine, Cysteine, SAM and Taurine Metabolism | cysteine | 1.78 | 0.60 |
| Amino Acid | Methionine, Cysteine, SAM and Taurine Metabolism | N-acetylmethionine | 1.70 | 0.50 |
| Amino Acid | Methionine, Cysteine, SAM and Taurine Metabolism | methionine | 1.20 | 0.70 |
| Amino Acid | Methionine, Cysteine, SAM and Taurine Metabolism | methionine sulfoxide | 1.16 | 0.83 |
| Amino Acid | Phenylalanine and Tyrosine Metabolism | phenylalanine | 1.14 | 0.71 |
| Amino Acid | Phenylalanine and Tyrosine Metabolism | tyrosine | 1.14 | 0.76 |
| Amino Acid | Polyamine Metabolism | spermidine | 8.91 | 0.26 |
| Amino Acid | Tryptophan Metabolism | tryptophan | 1.26 | 0.63 |
| Amino Acid | Urea cycle; Arginine and Proline Metabolism | argininosuccinate | 6.25 | 0.38 |
| Amino Acid | Urea cycle; Arginine and Proline Metabolism | proline | 1.16 | 0.48 |
| Carbohydrate | Aminosugar Metabolism | N-acetylneuraminate | 4.18 | 0.74 |
| Carbohydrate | Aminosugar Metabolism | erythronate* | 1.13 | 0.08 |
| Carbohydrate | Fructose, Mannose and Galactose Metabolism | mannitol/sorbitol | 2.25 | 0.50 |
| Carbohydrate | Glycolysis, Gluconeogenesis, and Pyruvate Metabolism | dihydroxyacetone phosphate (DHAP) | 9.87 | 0.59 |
| Carbohydrate | Nucleotide Sugar | UDP-N-acetylgalactosamine | 16.96 | 0.60 |
| Carbohydrate | Nucleotide Sugar | UDP-N-acetylglucosamine | 15.14 | 0.59 |
| Carbohydrate | Nucleotide Sugar | UDP-galactose | 12.83 | 0.14 |
| Carbohydrate | Nucleotide Sugar | UDP-glucose | 5.03 | 0.07 |
| Carbohydrate | Pentose Metabolism | ribonate | 1.31 | 0.20 |
| Cofactors and Vitamins | Ascorbate and Aldarate Metabolism | gulonic acid* | 2.71 | 0.11 |
| Cofactors and Vitamins | Nicotinate and Nicotinamide Metabolism | nicotinamide riboside | 2.66 | 0.14 |
| Cofactors and Vitamins | Nicotinate and Nicotinamide Metabolism | nicotinamide | 1.25 | 0.17 |
| Energy | TCA Cycle | malate | 8.33 | 0.58 |
| Energy | TCA Cycle | fumarate | 5.39 | 0.34 |
| Energy | TCA Cycle | alpha-ketoglutarate | 2.69 | 0.21 |
| Lipid | Carnitine Metabolism | deoxycarnitine | 3.25 | 0.10 |
| Lipid | Carnitine Metabolism | carnitine | 2.19 | 0.15 |
| Lipid | Fatty Acid Metabolism (also BCAA Metabolism) | butyrylcarnitine | 3.83 | 0.02 |
| Lipid | Fatty Acid Metabolism (also BCAA Metabolism) | propionylcamitine | 2.50 | 0.04 |
| Lipid | Fatty Acid Metabolism (also BCAA Metabolism) | methylmalonate (MMA) | 1.68 | 0.15 |
| Lipid | Fatty Acid Metabolism(Acyl Carnitine) | acetylcarnitine | 2.66 | 0.04 |
| Lipid | Fatty Acid, Dicarboxylate | 2-hydroxyglutarate | 3.06 | 0.07 |
| Lipid | Glycerolipid Metabolism | glycerol 3-phosphate | 14.17 | 0.66 |
| Lipid | Glycerolipid Metabolism | glycerophosphoglycerol | 5.32 | 0.27 |
| Lipid | Lysolipid | 1-oleoyl-GPC (18:1) | 2.74 | 0.46 |
| Lipid | Lysolipid | 1-stearoyl-GPC (18:0) | 1.21 | 0.53 |
| Lipid | Mevalonate Metabolism | 3-hydroxy-3-methylglutarate | 1.70 | 0.10 |
| Lipid | Neurotransmitter | acetylcholine | 19.50 | 0.23 |
| Lipid | Phospholipid Metabolism | glycerophosphoethanolamine | 37.37 | 0.07 |
| Lipid | Phospholipid Metabolism | glycerophosphorylcholine (GPC) | 17.82 | 0.10 |
| Lipid | Phospholipid Metabolism | choline phosphate | 12.15 | 0.27 |
| Lipid | Phospholipid Metabolism | choline | 1.45 | 0.72 |
| Nucleotide | Purine Metabolism, (Hypo)Xanthine/Inosine containing | inosine 5′-monophosphate (IMP) | 28.84 | 0.21 |
| Nucleotide | Purine Metabolism, (Hypo)Xanthine/Inosine containing | inosine | 5.14 | 0.11 |
| Nucleotide | Purine Metabolism, (Hypo)Xanthine/Inosine containing | hypoxanthine | 3.60 | 0.47 |
| Nucleotide | Purine Metabolism, (Hypo)Xanthine/Inosine containing | 2′-deoxyinosine | 2.38 | 0.67 |
| Nucleotide | Purine Metabolism, Adenine containing | 2′-deoxyadenosine 5′-monophosphate | 20.95 | 0.65 |
| Nucleotide | Purine Metabolism, Adenine containing | adenosine 5′-monophosphate (AMP) | 14.95 | 0.13 |
| Nucleotide | Purine Metabolism, Guanine containing | guanosine | 11.79 | 0.54 |
| Nucleotide | Purine Metabolism, Guanine containing | guanosine 5′- monophosphate (5′-GMP) | 8.66 | 0.74 |
| Nucleotide | Purine Metabolism, Guanine containing | 2′-deoxyguanosine | 3.12 | 0.61 |
| Nucleotide | Pyrimidine Metabolism, Cytidine containing | cytidine | 99.36 | 0.73 |
| Nucleotide | Pyrimidine Metabolism, Cytidine containing | cytidine 5′-monophosphate (5′-CMP) | 16.36 | 0.34 |
| Nucleotide | Pyrimidine Metabolism, Cytidine containing | 2′-deoxycytidine | 4.21 | 0.26 |
| Nucleotide | Pyrimidine Metabolism, Cytidine containing | 2′-deoxycytidine 5′-monophosphate | 1.54 | 0.06 |
| Nucleotide | Pyrimidine Metabolism, Orotate containing | orotidine | 5.30 | 0.06 |
| Nucleotide | Pyrimidine Metabolism, Orotate containing | N-carbamoylaspartate | 1.31 | 0.01 |
| Nucleotide | Pyrimidine Metabolism, Uracil containing | uridine | 25.03 | 0.66 |
| Nucleotide | Pyrimidine Metabolism, Uracil containing | uridine 5′-monophosphate (UMP) | 9.39 | 0.37 |
| Peptide | Gamma-glutamyl Amino Acid | gamma-glutamylthreonine* | 4.15 | 0.16 |
| Peptide | Gamma-glutamyl Amino Acid | gamma-glutamylmethionine | 2.02 | 0.45 |
| Peptide | Gamma-glutamyl Amino Acid | gamma-glutamylglutamine | 1.51 | 0.45 |

TABLE 3

HMT metabolites

NAD⁺
cAMP
cGMP
NADH
Xanthine
ADP-ribose
Mevalonic acid
UDP-glucose
Uric acid
NADP⁺
IMP
Sedoheptulose 7-phosphate
Glucose 6-phosphate
Fructose 6-phosphate
Fructose 1-phosphate
Galactose 1-phosphate
Glucose 1-phosphate
Acetoacetyl CoA
Acetyl CoA
Folic acid
Ribose 5-phosphate
CoA
Ribose 1-phosphate
Ribulose 5-phosphate
Xylulose 5-phosphate
Erythrose 4-phosphate
HMG CoA
Glyceraldehyde 3-phosphate
NADPH
Malonyl CoA
Phosphocreatine
XMP
Dihydroxyacetone phosphate
Adenylosuccinic acid
Fructose 1,6-diphosphate
6-Phosphogluconic acid
N-Carbamoylaspartic acid
PRPP
2-Phosphoglyceric acid
2,3-Diphosphoglyceric acid
3-Phosphoglyceric acid
Phosphoenolpyruvic acid
GMP
AMP
2-Oxoisovaleric acid
GDP
Lactic acid
ADP
GTP
Glyoxylate
ATP
Glycerol 3-phosphate
Glycolic acid
Pyruvic acid
N-Acetylglutamic acid
2-Hydroxyglutaric acid
Carbamoylphosphate
Succinic acid
Malic acid
2-Oxoglutaric acid
Fumaric acid
Citric acid
cis-Aconitic acid
Isocitric acid
Urea
Gly
Putrescine
Ala
Sarcosine
β-Ala
γ-Aminobutyric acid
N,N-Dimethylglycine
Choline
Ser
Carnosine
Creatinine
Pro
Val
Betaine
Thr
Homoserine
Betaine aldehyde
Cys
Hydroxyproline
Creatine
Leu
Ile
Asn
Ornithine
Asp
Homocysteine
Adenine
Hypoxanthine
Spermidine
Gln
Lys
Glu
Met
Guanine
His
Carnitine
Phe
Arg
Citrulline
Tyr
S-Adenosylhomocysteine
Spermine
Trp
Cystathionine
Adenosine
Inosine
Guanosine
Argininosuccinic acid
Glutathione (GSSG)
Glutathione (GSH)
S-Adenosylmethionine

### REFERENCES

All references listed in the instant disclosure, including but not limited to all patents, patent applications and publications thereof, scientific journal articles, and database entries (including but not limited to UniProt, EMBL, and GENBANK® biosequence database entries and including all annotations available therein) are incorporated herein by reference in their entireties to the extent that they supplement, explain, provide a background for, and/or teach methodology, techniques, and/or compositions employed herein. The discussion of the references is intended merely to summarize the assertions made by their authors. No admission is made that any reference (or a portion of any reference) is relevant prior art. Applicants reserve the right to challenge the accuracy and pertinence of any cited reference.

### Primary Reference List for the Specification

1. Fuchs, Y. & Steller, H. Programmed cell death in animal development and disease. Cell 147, 742-758 (2011).

2. Rothlin, C. V., Carrera Silva, E. A., Bosurgi, L. & Ghosh, S. TAM receptor signaling in immune homeostasis. Annu. Rev. Immunol. 33, 355-391 (2015).

3. Lindsten, T. et al. The combined functions of proapoptotic Bcl-2 family members bak and bax are essential for normal development of multiple tissues. Mol. Cell 6, 1389-1399 (2000).

4. Gregory, C.D., and Pound, J.D. Cell death in the neighbourhood: direct microenvironmental effects of apoptosis in normal and neoplastic tissues. J. Pathol. 223, 177-194 (2011).

5. Ryoo, H. D., Gorenc, T. & Steller, H. Apoptotic cells can induce compensatory cell proliferation through the JNK and the Wingless signaling pathways. Dev. Cell 7, 491-501 (2004).

6. Ke, F. F. S. et al. Embryogenesis and Adult Life in the Absence of Intrinsic Apoptosis Effectors BAX, BAK, and BOK. Cell 173, 1217-1230.e17 (2018).

7. Medina, C. B. & Ravichandran, K. S. Do not let death do us part: ‘find-me’ signals in communication between dying cells and the phagocytes. Cell Death Differ. 23, 979-989

8. Elliott, M. R. et al. Nucleotides released by apoptotic cells act as a find-me signal to promote phagocytic clearance. Nature 461, 282-286 (2009).

9. Van Opdenbosch, N. et al. Caspase-1 Engagement and TLR-Induced c-FLIP Expression Suppress ASC/Caspase-8-Dependent Apoptosis by Inflammasome Sensors NLRP1b and NLRC4. Cell Rep 21, 3427-3444 (2017).

10. Chekeni, F. B. et al. Pannexin 1 channels mediate ‘find-me’ signal release and membrane permeability during apoptosis. Nature 467, 863-867 (2010).

11. Poon, I. K. H. et al. Unexpected link between an antibiotic, pannexin channels and apoptosis. Nature 507, 329-334 (2014).

12. Good, M. E. et al. Pannexin 1 Channels as an Unexpected New Target of the Anti-Hypertensive Drug Spironolactone. Circ. Res. 122, 606-615 (2018).

13. Madeo, F., Eisenberg, T., Pietrocola, F. & Kroemer, G. Spermidine in health and disease. Science 359, eaan2788 (2018).

14. Liu, X. et al. PNPT1 Release from Mitochondria during Apoptosis Triggers Decay of Poly(A) RNAs. Cell (2018). doi:10.1016/j.cell.2018.04.017

15. Morioka, S. et al. Efferocytosis induces a novel SLC program to promote glucose uptake and lactate release. Nature 16, 907 (2018).

16. Wang, Y. et al. Mitochondrial Fission Promotes the Continued Clearance of Apoptotic Cells by Macrophages. Cell 171, 331-345.e22 (2017).

17. Perry, J. S. A. et al. Interpreting an apoptotic corpse as anti-inflammatory involves a chloride sensing pathway. Nat. Cell Biol. 21, 1532-1543 (2019).

18. Ipseiz, N. et al. The nuclear receptor Nr4a1 mediates anti-inflammatory effects of apoptotic cells. J. Immunol. 192, 4852-4858 (2014).

19. Chung, E. Y. et al. Interleukin-10 expression in macrophages during phagocytosis of apoptotic cells is mediated by homeodomain proteins Pbx1 and Prep-1. Immunity 27, 952-964 (2007).

20. Zaiss, D. M. W., Gause, W. C., Osborne, L. C. & Artis, D. Emerging functions of amphiregulin in orchestrating immunity, inflammation, and tissue repair. Immunity 42, 216-226 (2015).

21. Goessling, W. et al. Genetic interaction of PGE2 and Wnt signaling regulates developmental specification of stem cells and regeneration. Cell 136, 1136-1147 (2009).

22. Shayakul, C., Clémençon, B. & Hediger, M. A. The urea transporter family (SLC14): physiological, pathological and structural aspects. Mol. Aspects Med. 34, 313-322 (2013).

23. Jha, A. K. et al. Network integration of parallel metabolic and transcriptional data reveals metabolic modules that regulate macrophage polarization. Immunity 42, 419-430 (2015).

24. Scher, J. U. & Pillinger, M. H. The anti-inflammatory effects of prostaglandins. J. Investig. Med. 57, 703-708 (2009).

25. Korganow, A. S. et al. From systemic T cell self-reactivity to organ-specific autoimmune disease via immunoglobulins. Immunity 10, 451-461 (1999).

26. Veras, F. P. et al. Fructose 1,6-bisphosphate, a high-energy intermediate of glycolysis, attenuates experimental arthritis by activating anti-inflammatory adenosinergic pathway. Sci Rep 5, 15171 (2015).

27. Krupnick, A. S. et al. Orthotopic mouse lung transplantation as experimental methodology to study transplant and tumor biology. Nat Protoc 4, 86-93 (2009).

28. Stewart, S. et al. Revision of the 1996 working formulation for the standardization of nomenclature in the diagnosis of lung rejection. in 26, 1229-1242 (2007).

29. Kouskoff, V. et al. Organ-specific disease provoked by systemic autoimmunity. Cell 87, 811-822 (1996).

30. Evans, A. M., DeHaven, C. D., Barrett, T., Mitchell, M. & Milgram, E. Integrated, nontargeted ultrahigh performance liquid chromatography/electrospray ionization tandem mass spectrometry platform for the identification and relative quantification of the small-molecule complement of biological systems. Anal. Chem. 81, 6656-6667 (2009).

### Additional References

1. Peng, H. et al. Dimethyl fumarate inhibits dendritic cell maturation via nuclear factor κB (NF-κB) and extracellular signal-regulated kinase 1 and 2 (ERK1/2) and mitogen stress-activated kinase 1 (MSK1) signaling. J. Biol. Chem. 287, 28017-28026 (2012).

2. Fiedler, S. E. et al. Dimethyl fumarate activates the prostaglandin EP2 receptor and stimulates cAMP signaling in human peripheral blood mononuclear cells. Biochem. Biophys. Res. Commun. 475, 19-24 (2016).

3. Wilms, H. et al. Dimethylfumarate inhibits microglial and astrocytic inflammation by suppressing the synthesis of nitric oxide, IL-1beta, TNF-alpha and IL-6 in an in-vitro model of brain inflammation. J Neuroinflammation 7, 30 (2010).

4. Gude, D. R. et al. Apoptosis induces expression of sphingosine kinase 1 to release sphingosine-1-phosphate as a ‘come-and-get-me’ signal. FASEB J. 22, 2629-2638 (2008).

5. Littlewood-Evans, A. et al. GPR91 senses extracellular succinate released from inflammatory macrophages and exacerbates rheumatoid arthritis. J. Exp. Med. 213, 1655-1662 (2016).

6. Peruzzotti-Jametti, L. et al. Macrophage-Derived Extracellular Succinate Licenses Neural Stem Cells to Suppress Chronic Neuroinflammation. Cell Stem Cell 22, 355-368.e13 (2018).

7. Choi, Y. H. & Park, H. Y. Anti-inflammatory effects of spermidine in lipopolysaccharide-stimulated BV2 microglial cells. J. Biomed. Sci. 19, 31 (2012).

8. Guo, X. et al. Spermidine alleviates severity of murine experimental autoimmune encephalomyelitis. Invest. Ophthalmol. Vis. Sci. 52, 2696-2703 (2011).

9. Yang, Q. et al. Spermidine alleviates experimental autoimmune encephalomyelitis through inducing inhibitory macrophages. Cell Death Differ. (2016). doi:10.1038/cdd.2016.71

10. Madeo, F., Eisenberg, T., Pietrocola, F. & Kroemer, G. Spermidine in health and disease. Science 359, eaan2788 (2018).

11. Eisenberg, T. et al. Induction of autophagy by spermidine promotes longevity. Nat. Cell Biol. 11, 1305-1314 (2009).

12. Mondanelli, G. et al. A Relay Pathway between Arginine and Tryptophan Metabolism Confers Immunosuppressive Properties on Dendritic Cells. Immunity (2017). doi:10.1016/j.immuni.2017.01.005

13. Harrison, A. P., Tygesen, M. P., Sawa-Wojtanowicz, B., Husted, S. & Tatara, M. R. Alphaketoglutarate treatment early in postnatal life improves bone density in lambs at slaughter. Bone 35, 204-209 (2004).

14. Gudasheva, T. A. et al. Identification of a novel endogenous memory facilitating cyclic dipeptide cyclo-prolylglycine in rat brain. FEBSLett. 391, 149-152 (1996).

15. Ostrovskaia, R. U. et al. [Multicomponent antithrombotic effect of the neuroprotective prolyl dipeptide GVS-111 and its major metabolite cyclo-L-prolylglycine]. Eksp Klin Farmakol 65, 34-37 (2002).

16. Ostrovskaya, R. U. et al. Memory restoring and neuroprotective effects of the proline-containing dipeptide, GVS-111, in a photochemical stroke model. Behav Pharmacol 10, 549-553 (1999).

17. Wang, L., Albrecht, M. A. & Wurtman, R. J. Dietary supplementation with uridine-5′-monophosphate (UMP), a membrane phosphatide precursor, increases acetylcholine level and release in striatum of aged rat. Brain Res. 1133, 42-48 (2007).

18. Cansev, M., Watkins, C. J., van der Beek, E. M. & Wurtman, R. J. Oral uridine-5′-monophosphate (UMP) increases brain CDP-choline levels in gerbils. Brain Res. 1058, 101-108 (2005).

19. Teather, L. A. & Wurtman, R. J. Chronic administration of UMP ameliorates the impairment of hippocampal-dependent memory in impoverished rats. J. Nutr. 136, 2834-2837 (2006).

20. Wang, L., Pooler, A. M., Albrecht, M. A. & Wurtman, R. J. Dietary uridine-5′-monophosphate supplementation increases potassium-evoked dopamine release and promotes neurite outgrowth in aged rats. J. Mol. Neurosci. 27, 137-145 (2005).

21. Aitken, R. J., Mattei, A. & Irvine, S. Paradoxical stimulation of human sperm motility by 2-deoxyadenosine. J. Reprod. Fertil. 78, 515-527 (1986).

22. Fahlman, C. S., Bickler, P. E., Sullivan, B. & Gregory, G. A. Activation of the neuroprotective ERK signaling pathway by fructose-1,6-bisphosphate during hypoxia involves intracellular Ca2+ and phospholipase C. Brain Res. 958, 43-51 (2002).

23. Park, J.-Y. et al. Neuroprotection by fructose-1,6-bisphosphate involves ROS alterations via p38 MAPK/ERK. Brain Res. 1026, 295-301 (2004).

24. Veras, F. P. et al. Fructose 1,6-bisphosphate, a high-energy intermediate of glycolysis, attenuates experimental arthritis by activating anti-inflammatory adenosinergic pathway. Sci Rep 5, 15171 (2015).

25. Nunes, F. B. et al. An assessment of fructose-1,6-bisphosphate as an antimicrobial and anti-inflammatory agent in sepsis. Pharmacol. Res. 47, 35-41 (2003).

26. Cuesta, E. et al. Fructose 1,6-bisphosphate prevented endotoxemia, macrophage activation, and liver injury induced by D-galactosamine in rats. Crit. Care Med. 34, 807-814 (2006).

27. Schmidt, A. P., Lara, D. R., de Faria Maraschin, J., da Silveira Perla, A. & Onofre Souza, D. Guanosine and GMP prevent seizures induced by quinolinic acid in mice. Brain Res. 864, 40-43 (2000).

28. Soares, F. A. et al. Anticonvulsant effect of GMP depends on its conversion to guanosine. Brain Res. 1005, 182-186 (2004).

29. Haskó, G. & Cronstein, B. N. Adenosine: an endogenous regulator of innate immunity. Trends Immunol. 25, 33-39 (2004).

30. Smits, P. et al. Endothelial release of nitric oxide contributes to the vasodilator effect of adenosine in humans. Circulation 92, 2135-2141 (1995).

31. Sato, A. et al. Mechanism of vasodilation to adenosine in coronary arterioles from patients with heart disease. Am. J. Physiol. Heart Circ. Physiol. 288, H1633-40 (2005).

32. Yamaguchi, H., Maruyama, T., Urade, Y. & Nagata, S. Immunosuppression via adenosine receptor activation by adenosine monophosphate released from apoptotic cells. Elife 3, e02172 (2014).

33. Mabley, J. G. et al. Inosine reduces inflammation and improves survival in a murine model of colitis. Am. J. Physiol. Gastrointest. Liver Physiol. 284, G138-44 (2003).

34. Chen, J. et al. Inosine Released from Dying or Dead Cells Stimulates Cell Proliferation via Adenosine Receptors. Front Immunol 8, 504 (2017).

35. Haskó, G. et al. Inosine inhibits inflammatory cytokine production by a posttranscriptional mechanism and protects against endotoxin-induced shock. The Journal of Immunology 164, 1013-1019 (2000).

36. Whitmarsh, T. E. The treatment of multiple sclerosis with inosine. J Altern ComplementMed 15, 609-609 (2009).

37. Haskó, G., Sitkovsky, M. V. & Szabó, C. Immunomodulatory and neuroprotective effects of inosine. Trends Pharmacol. Sci. 25, 152-157 (2004).

38. Arase, T. et al. The UDP-glucose receptor P2RY14 triggers innate mucosal immunity in the female reproductive tract by inducing IL-8. J. Immunol. 182, 7074-7084 (2009).

39. Gao, Z.-G., Ding, Y. & Jacobson, K. A. UDP-glucose acting at P2Y14 receptors is a mediator of mast cell degranulation. Biochem. Pharmacol. 79, 873-879 (2010).

40. Jokela, T. A. et al. Extracellular UDP-glucose activates P2Y14 Receptor and Induces Signal Transducer and Activator of Transcription 3 (STAT3) Tyr705 phosphorylation and binding to hyaluronan synthase 2 (HAS2) promoter, stimulating hyaluronan synthesis of keratinocytes. J. Biol. Chem. 289, 18569-18581 (2014).

41. Li, W.-J., Mao, F.-X., Chen, H.-J., Qian, L.-H. & Buzby, J. S. Treatment with UDP-glucose, GDNF, and memantine promotes SVZ and white matter self-repair by endogenous glial progenitor cells in neonatal rats with ischemic PVL. Neuroscience 284, 444-458 (2015).

42. Zhang, D. et al. Effects of 3,4-dihydroxyacetophenone on the hypercholesterolemia-induced atherosclerotic rabbits. Biol. Pharm. Bull. 36, 733-740 (2013).

43. Olney, J. W. Brain lesions, obesity, and other disturbances in mice treated with monosodium glutamate. Science 164, 719-721 (1969).

44. During, M. J. & Spencer, D. D. Extracellular hippocampal glutamate and spontaneous seizure in the conscious human brain. Lancet 341, 1607-1610 (1993).

While the presently disclosed subject matter has been disclosed with reference to specific embodiments, it is apparent that other embodiments and variations of the presently disclosed subject matter may be devised by others skilled in the art without departing from the true spirit and scope of the presently disclosed subject matter.

1. A method of treating an inflammatory condition in a subject, the method comprising administering to the subject an effective amount of a plurality of metabolite compounds derived from an apoptotic cell, to thereby treat the inflammatory condition in the subject.

2. The method of claim 1, wherein the plurality of metabolite compounds derived from an apoptotic cell are formulated in a single composition.

3. The method of claim 1, wherein the plurality of metabolite compounds derived from an apoptotic cell are formulated in a pharmaceutically acceptable carrier.

4. The method of claim 1, wherein the plurality of metabolite compounds comprises three or more metabolite compounds derived from an apoptotic cell.

5. The method of claim 1, wherein the plurality of metabolite compounds are selected from the group consisting of spermidine, fructose 1,6-bisphosphate (FBP), dihydroxyacetone phosphate (DHAP), guanosine 5′-monophosphate (GMP), inosine 5′-monophosphate (IMP), and UDP-glucose and combinations thereof.

6. The method of claim 1, wherein the inflammatory condition is selected from the group consisting of arthritis, transplantation rejection, colitis, peritonitis, and atherosclerosis.

7. The method of claim 1, wherein the administering of the plurality of metabolite compounds derived from an apoptotic cell induces an anti-inflammatory response in a macrophage, a myeloid cell, a non-professional phagocyte, or any combination thereof.

8. The method of claim 1, further comprising administering an additional therapeutic agent to the subject.

9. A method of modulating gene expression in a cell, the method comprising contacting the cell with an effective amount of a plurality of metabolite compounds derived from an apoptotic cell, to thereby modulate gene expression in the cell.

10. The method of claim 9, wherein the plurality of metabolite compounds derived from an apoptotic cell are formulated in a single composition.

11. The method of claim 9, wherein the plurality of metabolite compounds derived from an apoptotic cell are formulated in a pharmaceutically acceptable carrier.

12. The method of claim 9, wherein the plurality of metabolite compounds comprises three or more metabolite compounds derived from an apoptotic cell.

13. The method of claim 9, wherein the plurality of metabolite compounds are selected from the group consisting of spermidine, fructose 1,6-bisphosphate (FBP), dihydroxyacetone phosphate (DHAP), guanosine 5′-monophosphate (GMP), inosine 5′-monophosphate (IMP), and UDP-glucose and combinations thereof.

14. The method of claim 9, wherein the cell is a cell in a subject.

15. The method of claim 14, wherein the gene expression is involved in a biological process selected from the group consisting of inflammation, wound healing, proliferation, and development.

16. A composition comprising, consisting essentially of, or consisting of an effective amount of a plurality of metabolite compounds derived from an apoptotic cell; and a carrier.

17. The composition of claim 16, wherein the carrier is a pharmaceutically acceptable carrier.

18. The composition of claim 16, wherein the plurality of metabolite compounds comprises three or more metabolite compounds derived from an apoptotic cell.

19. The composition of claim 16, wherein the plurality of metabolite compounds are selected from the group consisting of spermidine, fructose 1,6-bisphosphate (FBP), dihydroxyacetone phosphate (DHAP), guanosine 5′-monophosphate (GMP), inosine 5′-monophosphate (IMP), and UDP-glucose and combinations thereof.

20. The composition of claim 16, for use in treating an inflammatory condition, for use in preparing a medicament for treating an inflammatory condition; for use in modulating gene expression in a cell; and/or for use in preparing a medicament for modulating gene expression in a cell.

21. The composition of claim 20, wherein the inflammatory condition is selected from the group consisting of arthritis, transplantation rejection, colitis, peritonitis, and atherosclerosis.

22. The composition of claim 20 ,wherein the composition induces an anti-inflammatory response in a macrophage, a myeloid cell, a non-professional phagocyte, or any combination thereof.

23. The composition of claim 20, wherein the gene expression is involved in a biological process selected from the group consisting of inflammation, wound healing, proliferation, and development.

24. A method of classifying cell death, the method comprising providing a sample to be assessed; detecting in the sample a presence or an absence of a profile comprising a plurality of metabolite compounds; and classifying cell death in the sample based on the presence or the absence of the profile.

25. The method of claim 24, wherein the profile comprises a plurality of metabolite compounds derived from an apoptotic cell, optionally wherein the presence or the absence of the profile indicates whether the predominant type of cell death in the sample is apoptosis or is not apoptosis.

26. The method of claim 25, wherein the plurality of metabolite compounds comprises three or more metabolite compounds derived from an apoptotic cell.

27. The method of claim 24, wherein the profile comprises a metabolite compound selected from the group consisting of spermidine, fructose 1,6-bisphosphate (FBP), dihydroxyacetone phosphate (DHAP), guanosine 5′-monophosphate (GMP), inosine 5′-monophosphate (IMP), and UDP-glucose and combinations thereof.
